SEQUENCE LISTING (66 sequences)

SEQ ID NO: 1 - protein, 283 aa, Homo sapiens
  DISULFID 123: note = Interchain with beta-2-microglobulin
MASGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDCKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQP

SEQ ID NO: 2 - protein, 277 aa, Homo sapiens
  DISULFID 121: note = Interchain with beta-2-microglobulinInterchain with beta-2-microglobulin
MGSHSMRYFSTSVSRPGSGEPRFIAVGYVDDTQFVRFDSDAASQRMEPRAPWIEQERPEYWDQETRNVKAQSQTDRVDLGTLRGYYNQSEAGSHTIQIMYGCDVGSDGRFLRGYEQHAYDCKDYIALNEDLRSWTAADMAAQITQRKWEAARWAEQLRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 3 - protein, 277 aa, Homo sapiens
  DISULFID 121: note = Interchain with beta-2-microglobulin
MGSHSMRYFTTSVSRPGRGEPRFIAVGYVDDTQFVRFDSDAASQRMEPRAPWIEQEGPEYWDLQTRNVKAQSQTDRANLGTLRGYYNQSEAGSHTIQMMYGCDVGSDGRFLRGYRQDAYDCKDYIALNEDLRSWTAADMAAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 4 - protein, 277 aa, Homo sapiens
  DISULFID 121: note = Interchain with beta-2-microglobulin
MGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDCKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEP

SEQ ID NO: 5 - protein, 283 aa, Homo sapiens
  DISULFID 123: note = Interchain with beta-2-microglobulin
MASGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDCKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQP

SEQ ID NO: 6 - protein, 283 aa, Homo sapiens
  DISULFID 123: note = Interchain with beta-2-microglobulin
MASGSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDCKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPSSQP

SEQ ID NO: 7 - protein, 277 aa, Homo sapiens
  DISULFID 121: note = Interchain with beta-2-microglobulin
MGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDCKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 8 - protein, 279 aa, Homo sapiens
  DISULFID 123: note = Interchain with beta-2-microglobulin
MASGSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDCKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 9 - protein, 277 aa, Homo sapiens
  DISULFID 121: note = Interchain with beta-2-microglobulin
MGSHSMRYFYTAVSRPGRGEPRFISVGYVDDTQFVRFDSDAASPREEPRAPWIEQEGPEYWDRNTQICKTNTQTDRESLRNLRGYYNQSEAGSHTLQWMYGCDVGPDGRLLRGYNQFAYDCKDYIALNEDLSSWTAADTAAQITQRKWEAAREAEQLRAYLEGTCVEWLRRHLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 10 - protein, 277 aa, Homo sapiens
  DISULFID 121: note = Interchain with beta-2-microglobulin
MGSHSMRYFHTSVSRPGRGEPRFISVGYVDDTQFVRFDSDAASPRTEPRAPWIEQEGPEYWDRETQISKTNTQTYREDLRTLLRYYNQSEAGSHTIQRMSGCDVGPDGRLLRGYNQFAYDCKDYIALNEDLSSWTAADTAAQITQRKWEAARVAEQDRAYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 11 - protein, 277 aa, Homo sapiens
  DISULFID 121: note = Interchain with beta-2-microglobulin
MGSHSMRYFYTAMSRPGRGEPRFIAVGYVDDTQFVRFDSDAASPRTEPRAPWIEQEGPEYWDGETRNMKASAQTYRENLRIALRYYNQSEAGSHIIQRMYGCDLGPDGRLLRGHDQSAYDCKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 12 - protein, 281 aa, Homo sapiens
  DISULFID 121: note = Interchain with beta-2-microglobulin
MGSHSMRYFYTAMSRPGRGEPRFIAVGYVDDTQFVRFDSDAASPRMAPRAPWIEQEGPEYWDRETQISKTNTQTYRESLRNLRGYYNQSEAGSHTLQRMYGCDVGPDGRLLRGHDQSAYDCKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQS

SEQ ID NO: 13 - protein, 277 aa, Homo sapiens
  DISULFID 121: note = Interchain with beta-2-microglobulin
MGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDCKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWEP

SEQ ID NO: 14 - protein, 279 aa, Homo sapiens
  DISULFID 121: note = Interchain with beta-2-microglobulin
MGSHSMRYFSAAVSRPGRGEPRFIAMGYVDDTQFVRFDSDSASPRMEPRAPWVEQEGPEYWEEETRNTKAHAQTDRMNLQTLRGYYNQSEASSHTLQWMIGCDLGSDGRLIRGYERYAYDCKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQGS

SEQ ID NO: 15 - protein, 285 aa, Homo sapiens
  DISULFID 121: note = Interchain with beta-2-microglobulin
MGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDCKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPSGEEQRYTCHVQHEGLPQPLILRWEQSPQPTIPI

SEQ ID NO: 16 - protein, 271 aa, Homo sapiens
  DISULFID 117: note = Interchain with beta-2-microglobulin
MRTHSLRYFRLGVSDPIHGVPEFISVGYVDSHPITTYDSVTRQKEPRAPWMAENLAPDHWERYTQLLRGWQQMFKVELKRLQRHYNHSGSHTYQRMIGCELLEDGSTTGFLQYAYDCQDFLIFNKDTLSWLAVDNVAHTIKQAWEANQHELLYQKNWLEEECIAWLKRFLEYGKDTLQRTEPPLVRVNRKETFPGVTALFCKAHGFYPPEIYMTWMKNGEEIVQEIDYGDILPSGDGTYQAWASIELDPQSSNLYSCHVEHCGVHMVLQVP

SEQ ID NO: 17 - protein, 302 aa, Homo sapiens
  DISULFID 138: note = Interchain with beta-2-microglobulin
MGCLLFLLLWALLQAWGSAEVPQRLFPLRCLQISSFANSSWTRTDGLAWLGELQTHSWSNDSDTVRSLKPWSQGTFSDQQWETLQHIFRVYRSSFTRDVKEFAKMLRLSYPLELQVSAGCEVHPGNASNNFFHVAFQCKDILSFQGTSWEPTQEAPLWVNLAIQVLNQDKWTRETVQWLLNGTCPQFVSGLLESGKSELKKQVKPKAWLSRGPSPGPGRLLLVCHVSGFYPKPVWVKWMRGEQEQQGTQPGDILPNADETWYLRATLDVVAGEAAGLSCRVKHSSLEGQDIVLYWGGPIDKL

SEQ ID NO: 18 - protein, 100 aa, Homo sapiens
  DISULFID 31: note = Interchain with HLA-A, HLA-B, HLA-Ib, or non-classical alleles
MIQRTPKIQVYSRHPAENGKSNFLNCYVSGFCPSDIEVDLLKNGERIEKVEHSDLSFSKDWSFYLLYYTEFTPTEKDEYACRVNHVTLSQPKIVKWDRDM

SEQ ID NO: 19 - protein, 122 aa, Homo sapiens
  REPEAT 101..105: note = (GGGGS)n repeat where n=1 or greater
  DISULFID 31: note = Interchain with HLA-A, HLA-B, HLA-Ib, or non-classical alleles
MIQRTPKIQVYSRHPAENGKSNFLNCYVSGFCPSDIEVDLLKNGERIEKVEHSDLSFSKDWSFYLLYYTEFTPTEKDEYACRVNHVTLSQPKIVKWDRDMGGGGSGSLHHILDAQKMVWNHR

SEQ ID NO: 20 - protein, 118 aa, Homo sapiens
  REPEAT 101..105: note = (GGGGS)n repeat where n=1 or greater
  DISULFID 31: note = Interchain with HLA-A, HLA-B, HLA-Ib, or non-classical alleles
MIQRTPKIQVYSRHPAENGKSNFLNCYVSGFCPSDIEVDLLKNGERIEKVEHSDLSFSKDWSFYLLYYTEFTPTEKDEYACRVNHVTLSQPKIVKWDRDMGGGGSAHIVMVDAYKPTK

SEQ ID NO: 21 - protein, 294 aa, Homo sapiens
MGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDCKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPGSLHHILDAQKMVWNHR

SEQ ID NO: 22 - protein, 300 aa, Homo sapiens
MASGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDCKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPGSLHHILDAQKMVWNHR

SEQ ID NO: 23 - protein, 300 aa, Homo sapiens
MASGSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDCKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPSSQPGSLHHILDAQKMVWNHR

SEQ ID NO: 24 - protein, 294 aa, Homo sapiens
MGSHSMRYFTTSVSRPGRGEPRFIAVGYVDDTQFVRFDSDAASQRMEPRAPWIEQEGPEYWDLQTRNVKAQSQTDRANLGTLRGYYNQSEAGSHTIQMMYGCDVGSDGRFLRGYRQDAYDCKDYIALNEDLRSWTAADMAAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPGSLHHILDAQKMVWNHR

SEQ ID NO: 25 - protein, 294 aa, Homo sapiens
MGSHSMRYFSTSVSRPGSGEPRFIAVGYVDDTQFVRFDSDAASQRMEPRAPWIEQERPEYWDQETRNVKAQSQTDRVDLGTLRGYYNQSEAGSHTIQIMYGCDVGSDGRFLRGYEQHAYDCKDYIALNEDLRSWTAADMAAQITQRKWEAARWAEQLRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELGSLHHILDAQKMVWNHR

SEQ ID NO: 26 - protein, 298 aa, Homo sapiens
MGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDCKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSGSLHHILDAQKMVWNHR

SEQ ID NO: 27 - protein, 300 aa, Homo sapiens
MASGSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDCKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSGSLHHILDAQKMVWNHR

SEQ ID NO: 28 - protein, 298 aa, Homo sapiens
MGSHSMRYFYTAMSRPGRGEPRFIAVGYVDDTQFVRFDSDAASPRMAPRAPWIEQEGPEYWDRETQISKTNTQTYRESLRNLRGYYNQSEAGSHTLQRMYGCDVGPDGRLLRGHDQSAYDCKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSGSLHHILDAQKMVWNHR

SEQ ID NO: 29 - protein, 294 aa, Homo sapiens
MGSHSMRYFHTSVSRPGRGEPRFISVGYVDDTQFVRFDSDAASPRTEPRAPWIEQEGPEYWDRETQISKTNTQTYREDLRTLLRYYNQSEAGSHTIQRMSGCDVGPDGRLLRGYNQFAYDCKDYIALNEDLSSWTAADTAAQITQRKWEAARVAEQDRAYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPGSLHHILDAQKMVWNHR

SEQ ID NO: 30 - protein, 294 aa, Homo sapiens
MGSHSMRYFYTSVSRPGRGEPRFISVGYVDDTQFVRFDSDAASPREEPRAPWIEQEGPEYWDRNTQISKTNTQTYRENLRIALRYYNQSEAGSHTLQRMYGCDVGPDGRLLRGHNQFAYDCKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPGSLHHILDAQKMVWNHR

SEQ ID NO: 31 - protein, 294 aa, Homo sapiens
MGSHSMRYFYTAMSRPGRGEPRFIAVGYVDDTQFVRFDSDAASPRTEPRAPWIEQEGPEYWDGETRNMKASAQTYRENLRIALRYYNQSEAGSHIIQRMYGCDLGPDGRLLRGHDQSAYDCKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPGSLHHILDAQKMVWNHR

SEQ ID NO: 32 - protein, 296 aa, Homo sapiens
MGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDCKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWEPGSGGGLNDIFEAQKIEWHE

SEQ ID NO: 33 - protein, 294 aa, Homo sapiens
MGSHSMRYFSAAVSRPGRGEPRFIAMGYVDDTQFVRFDSDSASPRMEPRAPWVEQEGPEYWEEETRNTKAHAQTDRMNLQTLRGYYNQSEASSHTLQWMIGCDLGSDGRLIRGYERYAYDCKDYLALNEDLRSWTAADTAAQISKRKSEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQGSLHHILDAQKMVWNHR

SEQ ID NO: 34 - protein, 302 aa, Homo sapiens
MGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDCKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPSGEEQRYTCHVQHEGLPQPLILRWEQSPQPTIPIGSLHHILDAQKMVWNHR

SEQ ID NO: 35 - protein, 288 aa, Homo sapiens
MRTHSLRYFRLGVSDPIHGVPEFISVGYVDSHPITTYDSVTRQKEPRAPWMAENLAPDHWERYTQLLRGWQQMFKVELKRLQRHYNHSGSHTYQRMIGCELLEDGSTTGFLQYAYDCQDFLIFNKDTLSWLAVDNVAHTIKQAWEANQHELLYQKNWLEEECIAWLKRFLEYGKDTLQRTEPPLVRVNRKETFPGVTALFCKAHGFYPPEIYMTWMKNGEEIVQEIDYGDILPSGDGTYQAWASIELDPQSSNLYSCHVEHSGVHMVLQVPGSLHHILDAQKMVWNHR

SEQ ID NO: 36 - protein, 297 aa, Homo sapiens
MAEVPQRLFPLRSLQISSFANSSWTRTDGLAWLGELQTHSWSNDSDTVRSLKPWSQGTFSDQQWETLQHIFRVYRSSFTRDVKEFAKMLRLSYPLELQVSAGCEVHPGNASNNFFHVAFQCKDILSFQGTSWEPTQEAPLWVNLAIQVLNQDKWTRETVQWLLNGTCPQFVSGLLESGKSELKKQVKPKAWLSRGPSPGPGRLLLVCHVSGFYPKPVWVKWMRGEQEQQGTQPGDILPNADETWYLRATLDVVAGEAAGLSCRVKHSSLEGQDIVLYWGGGGGLNDIFEAQKIEWHE

SEQ ID NO: 37 - protein, 100 aa, Homo sapiens; identical to SEQ ID NO: 18

SEQ ID NO: 38 - protein, 9 aa, Homo sapiens
  SITE 1: note = FITC
KSDPIVAQY

SEQ ID NO: 39 - protein, 9 aa, Homo sapiens
  SITE 1: note = TAMRA
KLFGYPVYV

SEQ ID NO: 40 - protein, 9 aa, Homo sapiens
  SITE 1: note = TAMRA
KYNPIRTTF

SEQ ID NO: 41 - protein, 9 aa, Homo sapiens
  SITE 1: note = FITC
KLIDVFHQY

SEQ ID NO: 42 - protein, 9 aa, Homo sapiens
  SITE 1: note = FITC
KTFPPTEPK

SEQ ID NO: 43 - protein, 9 aa, Homo sapiens
  SITE 1: note = FITC
KPPIFIRRL

SEQ ID NO: 44 - protein, 9 aa, Homo sapiens
  SITE 1: note = FITC
KLRGRAYGL

SEQ ID NO: 45 - protein, 9 aa, Homo sapiens
  SITE 1: note = FITC
KHIPGDTLF

SEQ ID NO: 46 - protein, 9 aa, Homo sapiens
  SITE 1: note = FITC
KEDLRVSSF

SEQ ID NO: 47 - protein, 10 aa, Homo sapiens
  SITE 1: note = FITC
KSTLQEQIGW

SEQ ID NO: 48 - protein, 10 aa, Homo sapiens
  SITE 1: note = FITC
KQDIYRASYY

SEQ ID NO: 49 - protein, 9 aa, Homo sapiens
  SITE 1: note = FITC
KLPAKAPLL

SEQ ID NO: 50 - protein, 9 aa, Homo sapiens
  SITE 1: note = FITC
KYIHSANVL

SEQ ID NO: 51 - protein, 9 aa, Homo sapiens
  SIGNAL 6: note = L-beta-Phenylalanine
STAPGFLEY

SEQ ID NO: 52 - protein, 9 aa, Homo sapiens
  SITE 8: note = L-beta-Phenylalanine
KTFPPTEFK

SEQ ID NO: 53 - protein, 9 aa, Homo sapiens
FTSDYYQLY

SEQ ID NO: 54 - protein, 8 aa, Homo sapiens
LFGYPVYV

SEQ ID NO: 55 - protein, 9 aa, Homo sapiens
LLFGYPVYV

SEQ ID NO: 56 - protein, 9 aa, Homo sapiens
QYNPIRTTF

SEQ ID NO: 57 - protein, 9 aa, Homo sapiens
  SITE 8: note = L-beta-Phenylalanine
RPPIFIRFL

SEQ ID NO: 58 - protein, 9 aa, Homo sapiens
FLRGRAYJL

SEQ ID NO: 59 - protein, 9 aa, Homo sapiens
  SITE 8: note = L-beta-Phenylalanine
YHIPGDTFF

SEQ ID NO: 60 - protein, 9 aa, Homo sapiens
  SITE 7: note = L-beta-Phenylalanine
FEDLRVFSF

SEQ ID NO: 61 - protein, 10 aa, Homo sapiens
TSTLQEQIGW

SEQ ID NO: 62 - protein, 10 aa, Homo sapiens
ILDTAGKEEY

SEQ ID NO: 63 - protein, 9 aa, Homo sapiens
  SITE 8: note = L-beta-Phenylalanine
RLPAKAPFL

SEQ ID NO: 64 - protein, 9 aa, Homo sapiens
  SITE 8: note = L-beta-Phenylalanine
KYIHSANFL

SEQ ID NO: 65 - protein, 147 aa, Homo sapiens
MGSSGSLHHILDAQKMVWNHRSSGGGGSGGGGSGGGGSSGLVPRGSHMIQRTPKIQVYSRHPAENGKSNFLNCYVSGFCPSDIEVDLLKNGERIEKVEHSDLSFSKDWSFYLLYYTEFTPTEKDEYACRVNHVTLSQPKIVKWDRDM

SEQ ID NO: 66 - protein, 144 aa, Homo sapiens
MGSSVPTIVMVDAYKRYKSSGGGGSGGGGSGGGGSSGLVPRGSHMIQRTPKIQVYSRHPAENGKSNFLNCYVSGFCPSDIEVDLLKNGERIEKVEHSDLSFSKDWSFYLLYYTEFTPTEKDEYACRVNHVTLSQPKIVKWDRDM

(19) United States
(12) Patent Application Publication
(10) Pub. No.: US 2026/0097105 A1
(43) Pub. Date: Apr. 9, 2026

(54) SYSTEMS, FORMULATIONS AND METHODS FOR GENERATING UNIVERSAL PEPTIDE/MHC COMPLEXES WITH ENGINEERED DISULFIDE CONNECTING THE HEAVY AND LIGHT CHAINS

(71) Applicant: The Children's Hospital of Philadelphia, Philadelphia, PA (US)

(72) Inventor: Nikolaos SGOURAKIS, Philadelphia, PA (US)

(21) Appl. No.: 19/114,130

(22) PCT Filed: Sep. 28, 2023
(86) PCT No.: PCT/US2023/033968

§ 371 Date: Mar. 21, 2025

Related U.S. Application Data
(60) Provisional application No. 63/377,947, filed on Sep. 30, 2022.
(60) Provisional application No. 63/490,728, filed on Mar. 16, 2023.
(60) Provisional application No. 63/510,796, filed on Jun. 28, 2023.

(51) Int. Cl. A61K 39/00; A61K 39/385; A61P 35/00; C07K 14/47; C07K 14/74

(52) U.S. Cl. CPC A61K 39/001188; A61K 39/001191; A61K 39/385; A61P 35/00; C07K 14/4748; C07K 14/70539; A61K 2039/605; C07K 2319/00

(57) ABSTRACT

The present invention relates to engineering synthetic major histocompatibility complex (MHC) molecules for generating universal peptide/MHC complexes with engineered disulfide linkage(s) using structure-guided modeling and design and method for making and using the same.

### RELATED APPLICATIONS AND INCORPORATION BY REFERENCE

This application is a National Stage of International Application No. PCT/US2023/033968, filed 28 Sep. 2023, which claims benefit of and priority to Serial Nos. U.S. 63/377,947, filed 30 Sep. 2022, U.S. 63/490,728, filed 16 Mar. 2023, and U.S. 63/510,796, filed 28 Jun. 2023.

### FEDERAL FUNDING LEGEND

This invention was made with government support under Grant No. 5R01AI143997 awarded by the National Institutes of Health. The government has certain rights in the invention.

The foregoing applications and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE LISTING

The instant application contains a Sequence Listing which is being submitted in XML format and is hereby incorporated by reference in its entirety. Said XML copy, created on Sep. 22, 2023, is named H5679-00016_Sequence_Listing.xml and is 83,813 bytes in size.

### FIELD OF THE INVENTION

The present invention relates to engineering synthetic major histocompatibility complex (MHC) molecules for generating universal peptide/MHC complexes with engineered disulfide linkage(s) using structure-guided modeling and design.

### BACKGROUND OF THE INVENTION

The immune system can respond to a plethora of continuously evolving intracellular threats, such as viruses, pathogenic bacteria, and cancerous cells. Immune surveillance at the cellular level is dependent on distinguishing self-proteins, which are expressed by the host's own genes and facilitate physiological cell function, from aberrantly expressed proteins, expressed by the virulent genes of infectious agents or by the host's mutated oncogenes. In jawed vertebrates, this surveillance process is made possible by a complex intracellular processing system, enabled by the proteins of the Major Histocompatibility Complex (MHC).

Class I MHC (MHC-I) proteins are expressed in all nucleated cells, and they are implicated in aspects of most, if not all, adaptive immune responses. They function by detecting aberrantly expressed proteins and alerting the immune system to the presence of intracellular threats by interacting with specialized receptors on T cells and Natural Killer (NK) cells.

The preparation of recombinant Major Histocompatibility Complex class I molecules remains a very cumbersome, laborious and time-consuming process.

Citation or identification of any document in this application is not an admission that such document is available as prior art to the present invention.

### SUMMARY OF THE INVENTION

The invention relates to a complex which may comprise (a) a protein encoding any one of (i) a dsMHC sequence of any one of Tables 1-6 and/or (ii) a sequence in FIG. 2 or FIG. 9A with a G120 mutation and/or a H31 mutation and (b) a peptide or a ligand capable of eliciting an immune response.

In one embodiment, the peptide or ligand in (b) may comprise an epitope, advantageously an immune epitope, such as a tumor-associated epitope, a neoantigen or a cancer epitope. In a particularly advantageous embodiment, the peptide may be from the Cancer Genome Atlas (TCGA) mutant epitope library (see, e.g., https://www.cancer.gov/about-nci/organization/ccg/research/structural-genomics/tcga).

In one embodiment, the peptide or ligand in (b) may be a small molecule cancer metabolite or a peptide. In another embodiment, the peptide or ligand may be a small molecule metabolite, a peptide, or a lipid. In another embodiment, the peptide or ligand may be a cancer metabolite such as but not limited to hypoxanthine, guanine, cytidine, guanosine, 5-aminoimidazole-4-carboxamide-1-β-D-ribofuranoside (AICAR), uridine 5′-monophosphate (UMP), shikimic acid, 3-dehydroshikimic acid, uridine, L-kynurenine, venlafanxine, salicylic acid, salicyluric acid, guanosine-5′-monophosphate (GMP), adenosine 5′-monophosphate monohydrate (AMP), or cytidine 5′-monophosphate (CMP).

The invention also relates to a nucleic acid encoding a complex which may comprise (a) a protein encoding any one of (i) a dsMHC sequence of any one of Tables 1-6 and/or (ii) a sequence in FIG. 2 or FIG. 9A with a G120 mutation and/or a H31 mutation and (b) a peptide capable of eliciting an immune response. The nucleic acid may be a single nucleic sequence or a first nucleic acid encoding (i) a dsMHC sequence of any one of Tables 1-6 and/or (ii) a sequence in FIG. 2 or FIG. 9A with a G120 mutation and/or a H31 mutation and a second nucleic acid encoding a peptide capable of eliciting an immune response.

The invention also relates to a vector which may comprise a nucleic acid encoding a complex which may comprise (a) a protein encoding any one of (i) a dsMHC sequence of any one of Tables 1-6 and/or (ii) a sequence in FIG. 2 or FIG. 9A with a G120 mutation and/or a H31 mutation and (b) a peptide capable of eliciting an immune response. The vector may be a single vector encoding a single nucleic sequence or a first vector encoding a first nucleic acid encoding (i) a dsMHC sequence of any one of Tables 1-6 and/or (ii) a sequence in FIG. 2 or FIG. 9A with a G120 mutation and/or a H31 mutation and a second vector encoding a second nucleic acid encoding a peptide capable of eliciting an immune response.

The invention also encompasses a pharmaceutical composition which may comprise any of the herein disclosed complexes, nucleic acids, or vectors and a pharmaceutically acceptable carrier.

The invention also encompasses methods of treating or preventing a disease in a subject in need thereof, which may comprise administering to the subject a therapeutically effective amount of any of the herein disclosed complexes, nucleic acids, vectors or pharmaceutical compositions. Advantageously, the disease is cancer.

The invention also encompasses a use of any one of the herein disclosed complexes, nucleic acids, vectors or pharmaceutical compositions to screen a panel of candidate epitopic peptides to identify relevant peptide disease targets. In one embodiment, the disease is cancer, autoimmunity or an infectious disease.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

FIGS. 1A-1F: Disulfide-linked HLA-I molecules show enhanced thermal stabilities and peptide exchange kinetics relative to their wild-type (WT) counterparts.

FIG. 1A. HLA-A*02:01/TAX9/hβ₂m structure with G120 and H31 (PDB ID:TDUZ) mutated to cysteine. HLA-A*02:01 is annotated with alphas (α₁, α₂, α₂₋₁, and α₃), while hβ₂m is the horizontal beta sheet annotated with β₂m.

FIG. 1B. A schematic summary of the disulfide-linked HLA-I molecules with the desired tag on β₂m enables purification of the protein and sets the stage for tetramerization.

FIG. 1C. Distribution of Cβ-Cβ distances between mutated cysteines from G120 and H31 using pHLA-I/hβ₂m crystal structures with ≤3 Å resolution (n=215).

FIG. 1D. SEC traces of HLA-A*02:01/TAX8 (black) and dsA*02:01/TAX8 (pink). The arrow indicates the protein peaks and is further confirmed by SDS/PAGE analysis in reduced (R) or non-reduced (NR) conditions. Differential scanning fluorimetry (DSF) of HLA-A*02:01/TAX8 (black, Tₘ=41.6° C.) and dsA*02:01/TAX8 (pink, Tₘ=48.8° C.).

FIG. 1E. Thermal stabilities correlation of HLA-A*02:01/TAX8 and dsA*02:01/TAX8 loaded individually with 50 peptides from the Cancer Genome Atlas (TCGA) mutant epitope library. The solid line represents a conceptual 1:1 correlation (no difference in thermal stabilities).

FIG. 1F. Representative peptide exchange profiles of HLA-A*02:01/LFGYPVYV (TAX8) (SEQ ID NO: 54), HLA-A*02:01/LLFGYPVYV (TAX9) (SEQ ID NO: 55), and A*24:02/QYNPIRTTF (Phox2B) (SEQ ID NO: 56) in either disulfide-linked (ds) or WT form. Peptide exchange was monitored after adding the fluorescent peptides _{TAMRA}KLFGYPVYV (SEQ ID NO: 39) and _{TAMRA}KYNPIRTTF (SEQ ID NO: 40), respectively. The data were fitted to a monoexponential association model to determine apparent rate constants kₐₚₚ. Averages of three independent experiments are shown.

FIG. 2. Sequence alignment of representatives covering HLA-A and HLA-B supertypes as well as MHC-Ib and non-classical MHC-I. Full sequences of HLA-A*01:01, A*30:01, A*02:01, A*24:02, and A*03:01, HLA-B*07:02, B*08:01, B*14:02, B*37:01, B*58:01, and B*15:01, HLA-E*01:03, HLA-G*01:01, HLA-F*01:01, MR1, and CD1d were obtained from the IDP-IMGT/HLA database (Robinson, J. et al. IPD-IMGT/HLA Database. Nucleic Acids Research 48, D948-D955 (2020)). The HLA-A*02:01/hβ₂m crystal structure (PDB ID:1DUZ) were used to indicate secondary structures. Full sequence alignments were performed using ClustalOmega (Sievers, F. et al. Fast, scalable generation of high-quality protein multiple sequence alignments using Clustal Omega. Mol Syst Biol 7, 539 (2011)) and processed using ESPript (Robert, X. & Gouet, P. Deciphering key features in protein structures with the new ENDscript server. Nucleic Acids Research 42, W320-W324 (2014)) and the alignment between residue 115 and 165 was shown. Conserved amino acid G120 across HLA allotypes was indicated with a downward-facing arrow.

FIGS. 3A-3D. Structure-guided stabilization of suboptimal peptide-loaded HLA-A*02:01 by engineered disulfide between the HC and β₂m. A. Structure alignment and distribution of Cβ-Cβ distances between positions G120 of the HC and H31 of the β₂m derived from 215 pMHC-I/β₂m co-crystal structures with resolution values ≤3 Å. The structures of 52 distinct alleles are aligned by Cα atoms of α₁, α₂, and α₃ domains as ribbons. B. Structural model of HLA-A*02:01/β₂m/TAX9 (PDB ID:1DUZ) with G120 and H31 mutated to cysteines. HLA-A*02:01 HC is the structure encompassing the top-half of the image (G120C faces down from HLA-A*02:01 to form a disulfide bond with the H31C of hβ₂m) and hβ₂m is the structure encompassing the bottom-half of the image (H31C faces upward to form the disulfide bond with G120C of HLA-A*02:01). C. SEC traces of the WT (black) and the G120C/H31C open (pink) HLA-A*02:01/β₂m/TAX8. The downward-facing triangle arrowhead indicates the complex peaks and is further confirmed by SDS/PAGE analysis in reduced (R) or non-reduced (NR) conditions. DSF shows thermal stability curves of the WT in black (Tₘ=41.6° C.) and the open variant in pink (Tₘ=48.8° C.). The average of three technical replicates (mean) is plotted. D. Thermal stability correlation of the WT and open HLA-A*02:01/TAX8 loaded with each of 50 peptides from the Cancer Genome Atlas (TCGA) epitope library is shown in dots. The average of three technical replicates (mean) is plotted. The solid line represents a conceptual 1:1 correlation (no difference in thermal stabilities).

FIGS. 4A-4E. Disulfide-engineered pHLA-A*02:01 demonstrates substantial structural rearrangement. A-B. Calculated CSPs between the WT and open HLA-A*02:01/β₂m/MART1 are plotted as bar graphs across A. HC and B. β₂m amide backbone. A significance threshold of 0.05 ppm is determined that is 5-fold higher than the ¹H sensitivity of the NMR instrument. Residues with significant CSPs are highlighted in red, and exchange-broadened residues in the open HLA-A*02:01 relative to the WT are colored in yellow. Cysteine mutations (G120C and H31C) are indicated by an asterisk. C. Residues with CSPs above the significance threshold and exchange broadened in the open HLA-A*02:01/β₂m/MART1 are plotted as dark and light spheres for the amide, respectively, on a representative HLA-A*02:01/β₂m/MART1 crystal structure (PDB ID: 3MRQ). D-E. Enlarged images of D. hydrophobic residues near the disulfide bond and E. residues within the hydrogen network. Side chains are displayed and highlighted dark for significant CSPs and light for exchange-broadening.

FIGS. 5A-5D. Engineered disulfide stabilizes MHC-I at an open, peptide-receptive conformation. A. Percent unfolding defined by the normalized fluorescent intensity at 25° C. for the WT or open HLA-A*02:01/KILGFVFJV upon UV irradiation. The duration of UV irradiation is shown on the x-axis. Results of three technical replicates (mean±σ) are plotted. B. Percent deuterium uptake resolved to individual residues upon 600-second deuterium labeling for peptide-loaded (left) and empty (right) states are mapped onto the HLA-A*02:01 crystal structure (PDB ID: 1DUZ) for visualization. Light and dark colored regions indicate segments containing peptides with 100% ΔHDX (red—more deuteration) or 0% ΔHDX (blue—less deuteration), respectively; black indicates regions where peptides were not obtained for peptide-loaded and empty protein states. C. Association profiles of the fluorophore-conjugated peptide _{TAMRA}TAX9 to the WT or open HLA-A*02:01/TAX8, as indicated. Results of three replicates (mean) are plotted. D. Competitive binding of _{TAMRA}TAX9 to the WT or open HLA-A*02:01/TAX8 as a function of increasing peptide concentration, measured by fluorescence polarization. An irrelevant peptide, p29 (YPNVNIHNF), was used as a negative control.

FIGS. 6A-6E. Open MHC-I improves peptide exchange efficiency on a broad repertoire of HLA allotypes. A-B. Schematic summary of fitted kinetics obtained from FP analyses of peptide exchange. A. shows the dissociation of 40 nM _{TAMRA}TAX9/A02 in the presence of 1 uM unlabeled TAX9 and B. 40 nM _{TAMRA}TAX9 in different concentrations of TAX8/A02 (50, 75, 100, and 200 nM). C. The dissociation profiles of 40 nM WT or open _{TAMRA}TAX9/A02 in the presence of 1 μM unlabeled TAX9, and association profiles of 40 nM WT or open _{TAMRA}TAX9 in 50 nM TAX8/A02, as indicated. D. Linear correlations between the apparent rate constants K_{assoc.} and the concentrations of TAX8/A02. The extrapolation of the slope between K_{assoc.} and the concentrations of TAX8/A02 determines the apparent association rate k_{on.} E. Log-scale comparison of K_{assoc.} for the WT (black) or the open (pink) HLA-A*02:01, A*01:01, A*24:02, A*29:02, A*30:01, B*07:02, B*08:01, B*15:01, B*37:01, B*38:01, B*58:01, E*01:03, and G*01:01. The apparent rate constant K_{assoc.} was determined by fitting the raw trace to a monoexponential association model. NA indicates no fitted K_{assoc.}. Results of three technical replicates (mean±σ) are plotted.

FIGS. 7A-7D. Open HLA-A*02:01 enable effective T cell detection by reducing non-specific background staining compared to the WT. A. A schematic summary of the disulfide-linked HLA-I molecules with the desired BSA tag enables biotinylation and sets the stage for tetramerization. B. Staining of 1G4-transduced primary CD8+ T cells with PE-tetramers of open and WT HLA-A*02:01/NY-ESO-1(V), light and dark green, respectively. C. Staining of 1G4-transduced primary CD8+ T cells with PE-tetramers of open and WT HLA-A*02:01/NY-ESO-1(V), light and dark green, compared to open and WT HLA-A*02:01 loaded with a non-specific peptide, light and dark blue. D. Staining of 1G4-transduced primary CD8+ T cells with PE-tetramers of open HLA-A*02:01 loaded with different NY-ESO-1 peptides SLLMWITQV (light green), SLLMWITQVC (orange), and SLLMWITQA (purple).

FIG. 8. A graphic depiction of the free energy landscape for MHC-I peptide exchange. WT and open MHC-I molecules loaded with a moderate affinity placeholder peptide can spontaneously generate empty molecules. Open MHC-I enhanced peptide exchange kinetics by lowering the activation free energy of generating empty molecules and stabilizing these receptive molecules in a prolonged open conformation without influencing the binding affinities and thermal stabilities.

FIGS. 9A-9B. Sequence alignment of distinct HLA allotypes across HLA-A, B, and C as well as HLA-Ib. A. Sequence alignment of the HLA representatives extracted from Protein data bank, covering HLA-A and HLA-B supertypes as well as HLA-Ib, PDB ID as indicated. B. Seq2logo visualization of the sequence alignment for 75 distinct HLA allotypes with >1% global population frequency shows a conserved residue G120. Sequence weighting used clustering, pseudo count with a weight of 0, and Kullback-Leibler logotype. The percentage frequency of amino acids on a specific position higher than 10% is shown on the positive y-axis, and less than 10% amino acids on the negative y-axis. Allele sequences were derived from the IPD-IMGT/HLA and the alignment was performed using ClustalOmega.

FIGS. 10A-10B. Overlay of the WT and open MHC-I NMR spectra reveal substantial backbone chemical shift changes. 2D ¹H-¹⁵N TROSY spectra of [¹H, ¹³C, ¹⁵N]-labeled A. HC (HLA-A*02:01) refolded with unlabeled light chain (β₂m) and MART1 (ELAGIGILTV), or B. β₂m bound to unlabeled HC and MART1. Spectra representing the WT complex, collected at 800 MHz ¹H magnetic field, are colored in gray, overlayed by the open complex spectra, collected at 600 MHz ¹H magnetic field, in pink. 2D TROSY data for the open MHC-I were also collected at 800 MHz ¹H magnetic field to verify that these results are consistent across magnetic fields, shown in FIGS. 11A-11B. All data were collected with identical buffer conditions (20 mM sodium phosphate, pH 7.2, and 150 mM NaCl) and at the RT (25° C.).

FIGS. 11A-11B. Overlay of NMR spectra for the open MHC-I confirms the same backbone chemical shifts regardless of the magnetic field. ¹H-¹⁵N TROSY data collected for the open HLA-A*02:01/β₂m/MART1 at both 600 MHz (orange) and 800 MHz (red) for [¹H, ¹³C, ¹⁵N]-labeled HC refolded with β₂m and MART1 (top), or β₂m bound to unlabeled HC and MART1 (bottom). Additional peaks in the spectra collected at 800 MHz are largely due to protein degradation and do not affect the chemical shifts corresponding to the protein backbone. All data were collected with identical buffer conditions (20 mM sodium phosphate, pH 7.2, and 150 mM NaCl) and at RT (25° C.).

FIG. 12. Percent deuterium uptake resolved to individual peptide fragments. Peptide segments of 38-60, α₁ 60-84, 127-132, α₂₋₁ 139-156, and β₂m 35-56 are plotted for each exposure time (0, 20, 180, and 600 s) and protein states. The plots reveal the local HDX profiles of HLA-A*02:01 for the states of peptide-loaded (black, refolded with KILGFVFJV) and empty (pink, UV irradiation for 40 minutes at 4° C.).

FIG. 13. Binding of _{TAMRA}TAX9 by the WT or open TAX8/A02. Peptide exchange measured by fluorescence polarization (mP) of 40 nM _{TAMRA}TAX9 as a function of the WT or open TAX8/A02 concentrations. Individual traces were fit to a monoexponential association model to determine apparent rate constants K_{assoc.} plotted in FIG. 6D. Results of three replicates (mean) are plotted.

FIGS. 14A-14L. Peptide exchange kinetics of the open vs. WT HLA allotypes. A-L, The association profiles of fluorophore-conjugated peptides _{FITC}KSDPIVAQY (SEQ ID NO: 38), _{TAMRA}KYNPIRTTF (SEQ ID NO: 39), _{FITC}KLIDVFHQY (SEQ ID NO: 41), _{FITC}KTFPPTEPK (SEQ ID NO: 42), _{FITC}KPPIFIRRL (SEQ ID NO: 43), _{FITC}KLRGRAYGL (SEQ ID NO: 44), _{FITC}KQDIYRASYY (SEQ ID NO: 48) _{FITC}KEDLRVSSF (SEQ ID NO: 46), _{FITC}KHIPGDTLF (SEQ ID NO: 45), _{FITC}KSTLQEQIGW (SEQ ID NO: 47), _{FITC}KLPAKAPLL (SEQ ID NO: 49), and _{FITC}KYIHSANVL (SEQ ID NO: 50) to the open (pink) and WT (black) HLA-A. A*01:01/STAPG(βF)LEY (SEQ ID NO: 51), B. A*24:02/QYNPIRTTF (SEQ ID NO:56), C. A*29:02/FTSDYYQLY (SEQ ID NO: 53), D. A*30:01/KTFPPTE(βF)K (SEQ ID NO: 52), E. B*07:02/RPPIFIR(βF)L (SEQ ID NO: 57), F. B*08:01/FLRGRAYJL (SEQ ID NO: 58), G. B*15:01/ILDTAGKEEY (SEQ ID NO: 62), H. B*37:01/FEDLRV(βF)SF (SEQ ID NO: 60), I. B*38:01/YHIPGDT(βF)F (SEQ ID NO: 58), J. B*58:01/TSTLQEQIGW (SEQ ID NO: 61), K. E*01:03/RLPAKAP(βF)L (SEQ ID NO: 63), and L. G*01:01/KYIHSAN(βF)L (SEQ ID NO: 64). Results of three replicates (mean) are plotted. The data were fitted to a monoexponential association model to determine apparent rate constants K_{assoc.} NA means the K_{assoc.} cannot be fitted. Results of three replicates (mean±σ) are plotted.

FIGS. 15A-15B. Selected T1D epitopes demonstrated the same IC50 profiles for the WT or open MHC-I. A-B, The IC₅₀ profiles extracted from the association profiles of A. _{TAMRA}KLFGYPVYV (SEQ ID NO: 39) binding to HLA-A*02:01/TAX8 and B. _{FITC}KLIDVFHQY (SEQ ID NO: 41) binding to HLA-A*29:02/FTSDYYQLY (SEQ ID NO: 53) in a concentration gradient of a competitor HLVEALYLV and ALIDVFHQY peptides, respectively. IC₅₀ values were determined by fitting a log(inhibitor) vs. response (three parameters) curve. Results of three replicates (mean±σ) are plotted.

FIGS. 16A-16D. Flow cytometry gating strategy of CD8+ T cells transduced with 1G4. Previously transduced or non-transduced primary human CD8+ T cells were thawed and recovered prior to tetramer staining. Cells were first sorted by side and forward scatter (SSC-A and FSC-A) followed by single cell isolation (SSC-A versus SSC-H plot). Gating for live cells was determined by Sytox blue staining and transduction efficiency was determined by staining with an anti-Vβ13.1-APC antibody (Miltenyi Biotec). Gates are shown in black and the percentages of events that are gated in parentheses. Acquisition was performed on CytoFLEX LX (Beckman Coulter) and the data analyzed by FlowJo v10.8.1.

FIGS. 17A-17E. Disulfide-engineered open MR1 and HLA-F*01:01 form stable protein complexes. A. Chemical structures of MR1 ligands DCF and Ac-6-FP. B-C. SEC traces of the WT (black) and open (pink) MR1 C262S/B. DCF and C. Ac-6-FP. D. Melting temperature (Tₘ, ° C.) obtained from DSF of the WT (black) and open (pink) MR1 C262S loaded with DCF or Ac-6-FP. The black triangle arrowhead indicates the protein complexes, which are further confirmed by SDS/PAGE analysis in reduced (R) or non-reduced (NR) conditions. Results of three technical replicates (mean±σ) are plotted. E. SEC traces of the WT (black) and open (pink) HLA-F*01:01/β₂m. The triangle arrowheads indicate the complex peaks and are further confirmed by SDS/PAGE analysis in reduced (R) or non-reduced (NR) conditions.

FIG. 18. CD8+ T cells co-staining. Cells were stained and sorted by anti-human TCR Vα7.2 antibody, anti-human CD161 antibody, double negative NIH MR1/6-FP, NIH MR1/5-OP-RU, open MR1/5-OP-RU, and WT MR1/5-OP-RU.

FIG. 19. Flow cytometry gating strategy of CD8+ T cells. Primary human CD8+ T cells were gated by side and forward scatter (SSC-A and FSC-A) followed by single cell isolation (FSC-A versus FSC-H plot). Gating for live CD8+ cells was determined by Sytox blue staining and an anti-human CD8α antibody, and TRAV1-2+ MAIT cells were determined by staining with an anti-human TCR Vu7.2 antibody and anti-human CD161 antibody. Gates are shown in black, and the percentages of events are gated in parentheses. The acquisition was performed on CytoFLEX LX (Beckman Coulter), and the data were analyzed by FlowJo v10.8.1.

FIGS. 20A-20E. N-terminally tagged β₂m are capable of complex formation. (A) SEC profile showing an overlay of open A02 with a BSP-tag fused to the C-terminus of the heavy chain (gray), a BSP-tag fused to the N-terminus of β₂m (pink), and a SpyTag fused to the N-terminus of β₂m (blue). (B) Coomassie-stained SDS-PAGE of open A02/BSP-open β₂m/KV9-βF complex under reducing and non-reducing conditions. (C) Coomassie-stained SDS-PAGE of open A02/SpyTag-open β₂m/KV9-βF complex under reducing and non-reducing conditions. (D) Conjugation of Streptavidin with open A02/BSP-open β₂m/KV9-βF. (E) Conjugation of SpyCatcher003-mi3 with open A02/SpyTag-open β₂m/KV9-βF at various ratios. Reactions were performed at 4° C. overnight and analyzed using SDS-PAGE with Coomassie staining.

FIGS. 21A-21C. Three cancer metabolites bind to MR1 at low millimolar binding affinities. A-B. Chemical structure of MR1-restricted known ligand floxuridine and tetrahydroxy-1,4-quinone hydrate and cancer metabolites Uridine 5′-monophosphate (UMP), 3-Dehydroshikimic acid (3DA), and shikimic acid (SKA). Chemicals sharing the same color background show structure similarity. C. Titration curves of cancer metabolites binding to MR1. Each data point represents plateau polarization (mP) values from three independent experiments performed in triplicate. Mean values are plotted with SD represented in error bars.

FIGS. 22A-22B. Competitive Binding of cancer metabolites and known ligands to open MR1. Ligand exchange measured by fluorescence polarization (miP) of 10 nM JMY20 and 200 nM DCF-loaded open MR1 in the presence of a serial dilution of cancer metabolites, as indicated. Individual traces were fit to a mono-exponential association model to determine plateau polarization (mP) values plotted in FIG. 21C and FIG. 22B. Results of three replicates (mean values) are plotted. B. Competitive binding of JMY20 to MR1 in the presence of known ligands DCF and acetyl-6-formylpterin (Ac-6-FP), and the IC₅₀ profiles extracted from the association profiles. IC₅₀ values were determined by fitting a log (inhibitor) vs. response (three parameters) curve. Results of three replicates (mean±SD) are plotted.

FIGS. 23A-C. Identification of MAIT cells from PBMCs using open MR1 tetramers exchanged for 5-OP-RU. A-B. Approximately 5×10⁵ human PBMCs were stained with 2 μg ml⁻¹ of Buffer (no tetramer), WT MR1/5-OP-RU (refolded, NIH), WT MR1/6-FP(refolded, NIH), open MR1/DCF, open MR1/5-OP-RU(exchanged) and open MR1/6-FP(exchanged) for 30 min at 4° C. in the dark. C. Approximately 5×10⁵ human PBMCs were stained with 20 μg ml⁻¹ open MR1/DCF and open MR1/5-OP-RU(exchanged). Cells were co-stained and gated with CD8+BV605, CD161-FITC, and Valpha7.2 (BD) for 30 min at 4° C. Cells were then washed three times with 200 μl of FACS wash (2% fetal bovine serum in PBS) and resuspended in 200 μl of FACS wash before acquisition of data on a Cytoflex LX. Data were analyzed using FlowJo analysis software. All tests are in technical triplicates. To prepare exchanged WT MR1 tetramer, refolded MR1/DCF was exchanged with 5-OP-RU or 6-FP in 10-fold molar excess (7 uM: 70 uM) and loaded in a 1:4 molar ratio with PE tetramers.

FIG. 24 Designed interchain disulfide bond does not influence TCR binding affinity to HLA-A*02:01. Representative SPR sensorgrams of various concentrations of TCR NYESO-S1 flowed over streptavidin chip channel coupled with the WT and open HLA-A*02:01/SLLMWITQV. HLA-A*02:01/SLLMAITQV served as a negative control coupled on the reference channel. The concentrations of analyte for sensorgrams are noted. KD, equilibrium constant, is fitted by saturation fit. Data are mean±SD, where n=3 technical replicates. RU, resonance units.

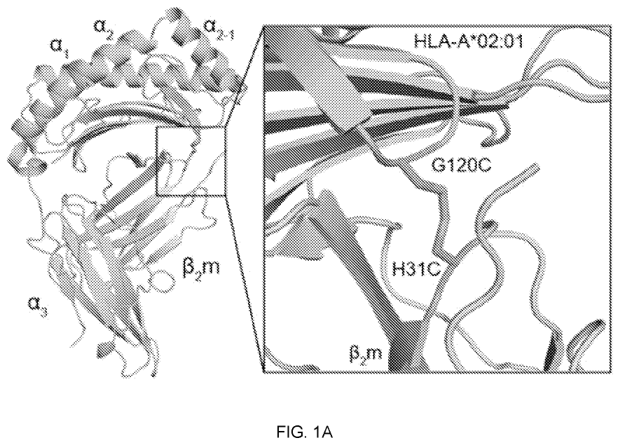

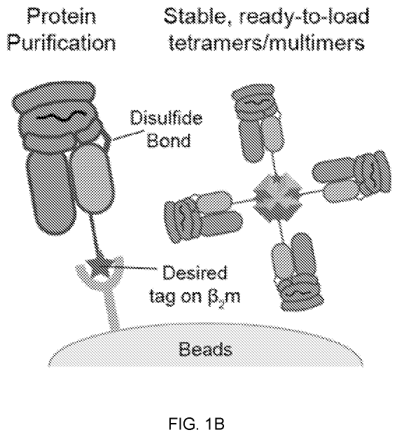

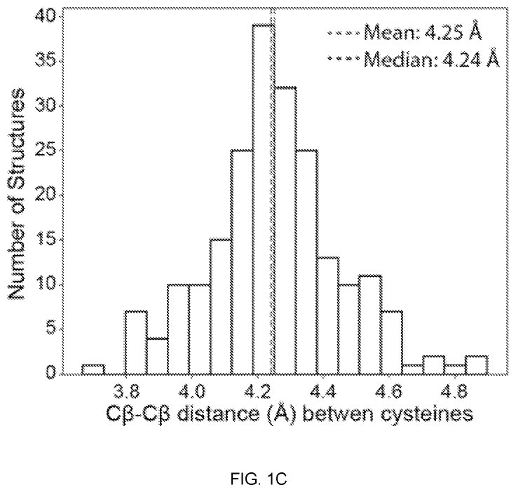

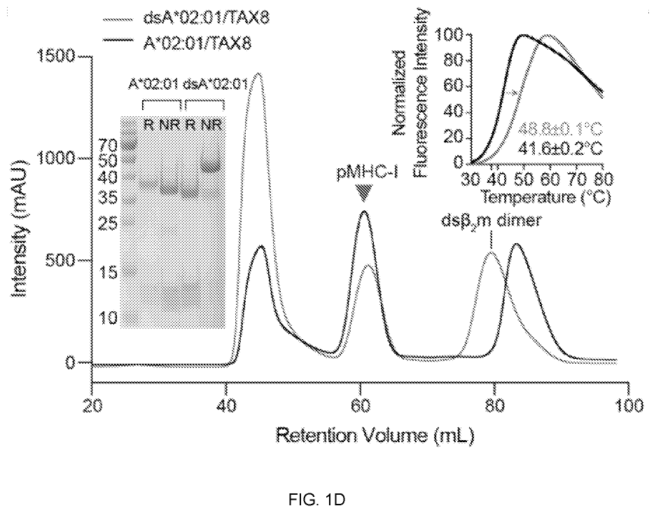

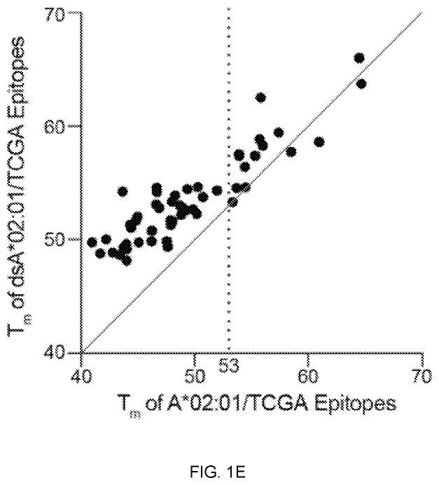

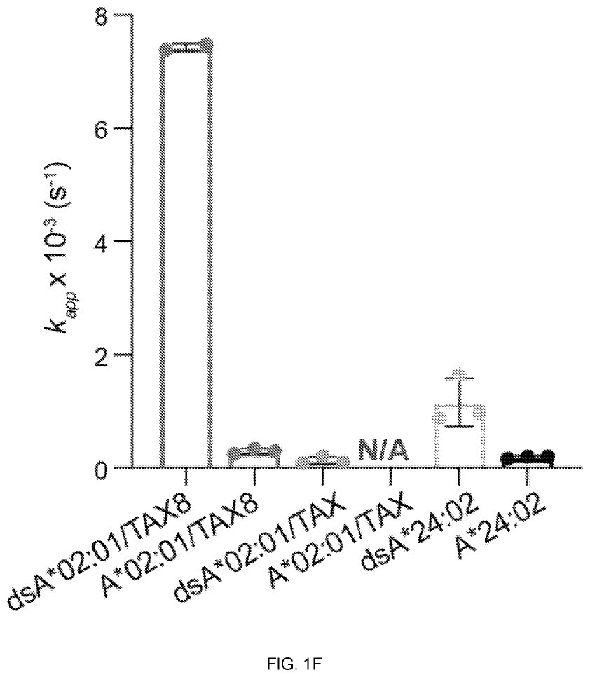

TABLE 1. Sequences of allotype representatives covering HLA-A supertypes, HLA-B supertypes, HLA-Ib, and non-classical MHC.

TABLE 2. The DSF determines thermal stabilities for Cancer Genome Atlas (TCGA) epitope library. Tₘ of individual peptides from the TCGA epitope library loaded on WT or open HLA-A*02:01 were measured via peptide exchange. Tₘ was determined by three technical replicates. The high-affinity HLA-A*02:01-restricted TAX9 peptide and refolded TAX9/A02 molecules were used as positive controls, and the irrelevant peptide p29 was used as a negative control.

TABLE 3. A summary of open MHC-I and β₂m sequences used in the study. Below are the protein sequences for the representative alleles from each HLA supertype. Cysteine mutations are colored in red.

TABLE 4. A summary of fluorophore-labeled peptides used in Example 2.

TABLE 5. A table summarizes the designed placeholder peptides and the Tₘ. Melting temperatures (Tₘ) were determined for the WT and mutant HLA allotype representatives. Each allotype was refolded with a selected placeholder peptide and measured for its Tₘ by three technical replicates.

TABLE 6. Sequences of the N-terminally tagged β₂m.

TABLE 7. Summary of cancer metabolites.

### DETAILED DESCRIPTION OF THE INVENTION

Applicants provide a method for designing disulfide-linked MHC molecules, where the heavy chain is covalently linked to the light chain. Applicants provide several designs encompassing multiple classical HLA (Human Leucocyte Antigen, the MHC in humans) super types, as well as for the non-classical MHC molecules MR1 and CD1, which demonstrates that this is a general approach.

Some of the advantages and applications of these engineered molecules include:
1) Increased stability and shelf-life of pMHC tetramers for applications in vitro and ex vivo.
2) More efficient and cost-effective workflow for making tetramers, using a universal β₂m protein tag.
3) Stabilization of complexes with lower-affinity peptide antigens and
4) Enables exchange of placeholder for high-affinity peptide antigens, without the use of molecular chaperones.

The present invention is described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. One skilled in the art will recognize that the systems and devices of embodiments of the invention can be used with any of the methods of the invention and that any methods of the invention can be performed using any of the systems and devices of the invention. Embodiments comprising various features may also consist of or consist essentially of those various features. Other embodiments of the invention will be apparent to those skilled in the art from consideration of the specification and practice of the invention. The description of the invention provided is merely exemplary in nature and, thus, variations that do not depart from the essence of the invention are intended to be within the scope of the invention.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Unless otherwise defined, all technical and scientific terms used herein have the same meaning as would be commonly understood or used by one of ordinary skill in the art encompassed by this technology and methodologies.

Protein sequences for the representative alleles of each HLA supertype, HLA-Ib alleles, and non-classical alleles are provided in any one of Tables 1-6. In TABLE 1, one residue in the sequence is underlined, this denotes the residue considered residue #1 in naming these mutations and the bold underlined residue is the cysteine mutation.

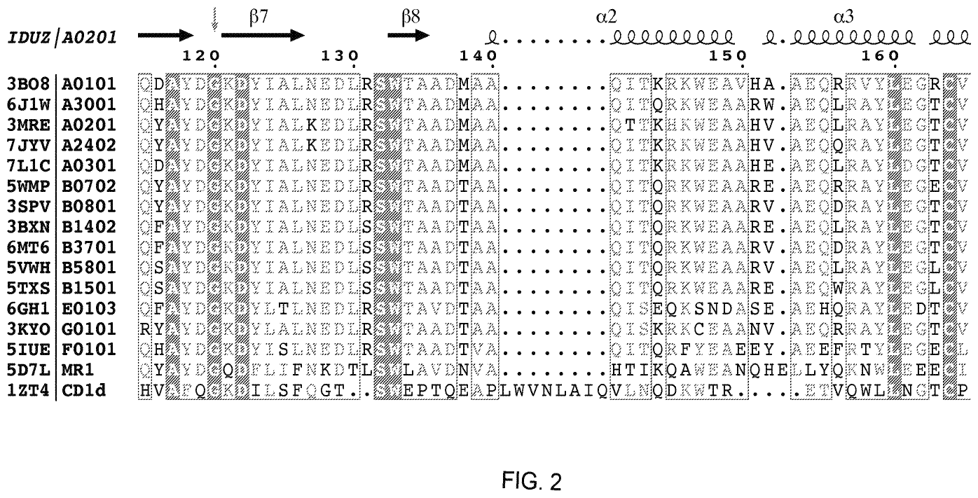

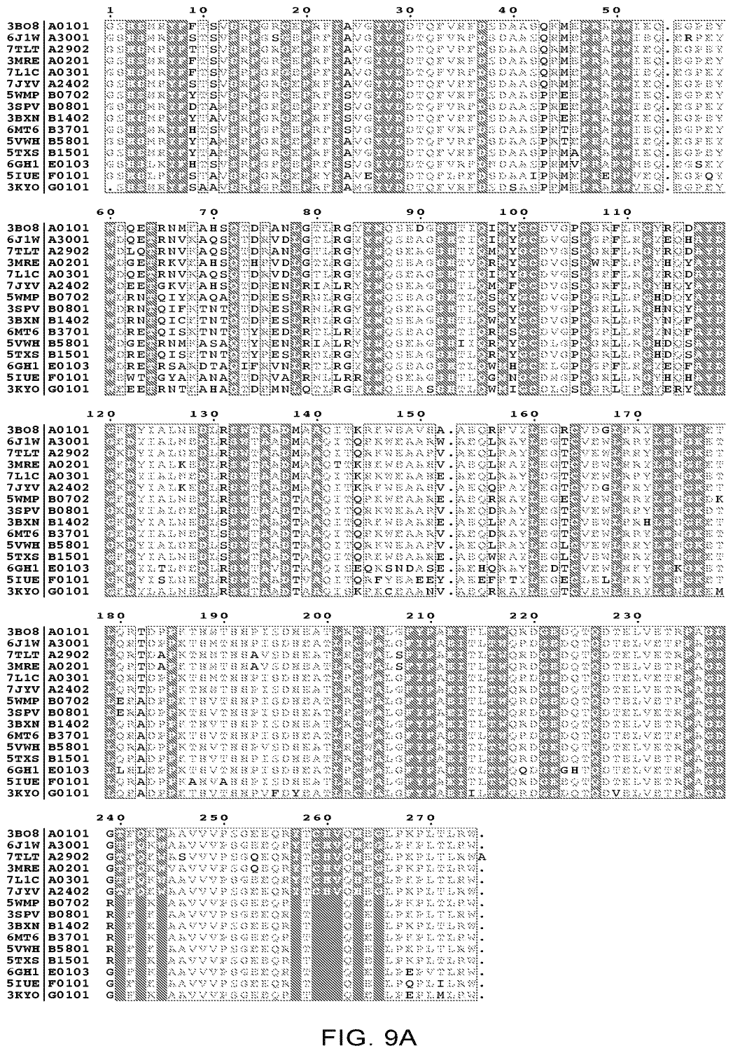

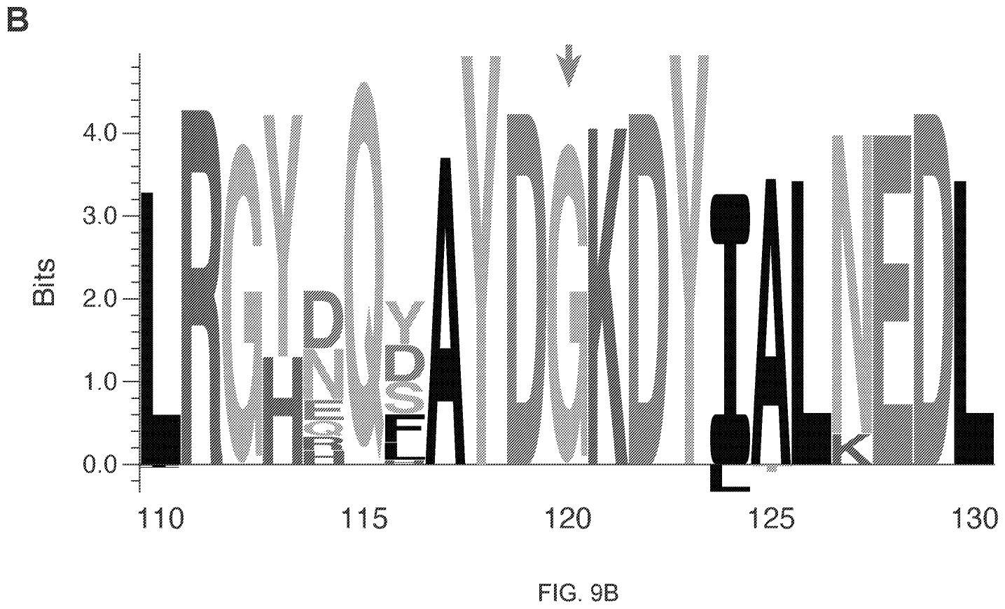

The present invention encompasses proteins of the dsMHC sequences of any one of Tables 1-6 and/or a sequence in FIG. 2 or FIG. 9A as well as the nucleic acids encoding the same.

The present invention encompasses a complex which comprises, consists essentially of or consists of (a) one or more proteins of the dsMHC sequences of any one of Tables 1-6 and/or a sequence in FIG. 2 or FIG. 9A as well as one or more nucleic acid(s) encoding the same and (b) one or more peptides capable of eliciting an immune response as well as one or more nucleic acid(s) encoding the same. Advantageously, the peptide comprises an epitope, advantageously an immune epitope, such as a tumor-associated epitope, a neoantigen or a cancer epitope. In a more advantageous embodiment, the peptide is from the Cancer Genome Atlas (TCGA) mutant epitope library (see, e.g., https://www.cancer.gov/about-nci/organization/ccg/research/structural-genomics/tcga).

In order for a peptide to elicit a cellular immune response, it may bind to an MHC molecule. This process depends on the allele of the MHC molecule and on the amino acid sequence of the peptide. Usually, MHC class I-binding peptides have a length of 8-10 residues, and their sequences contain two conserved residues (“anchors”) interacting with the corresponding binding groove of the MHC molecule.

In order for the immune system to be able to elicit an effective CTL response against tumor-derived peptides, such peptides may bind to certain MHC class I molecules being expressed by tumor cells, and may be recognized by T cells bearing specific T cell receptors (TCR).

The complex of the invention may be isolated and/or in a substantially pure form. For example, the complex may be provided in a form which is substantially free of other peptides or proteins. It should be noted that in the context of the present invention, the term “MHC molecule” includes recombinant MHC molecules, non-naturally occurring MHC molecules and functionally equivalent fragments of MHC, including derivatives or variants thereof, provided that peptide binding is retained. For example, MHC molecules may be fused to a therapeutic moiety, attached to a solid support, in soluble form, attached to a tag, biotinylated and/or in multimeric form. The peptide may be covalently attached to the MHC.

The disulfide (ds) mutant heavy chain and ds mutant light chains may be synthesized by mutating a suitable residue to cysteine to enable the formation of an internal disulfide bond. An alignment of all known high-resolution pMHC-I structures with distinct sequences (n=54) from a previously developed database HLA3DB was performed using ClustalOmega(Sievers, F. et al. Fast, scalable generation of high-quality protein multiple sequence alignments using Clustal Omega. Mol Syst Biol 7, 539 (2011)) and processed using ESPript (Robert, X. & Gouet, P. Deciphering key features in protein structures with the new ENDscript server. Nucleic Acids Research 42, W320-W324 (2014)). Conserved regions were identified across different HLA allotypes.

Using one of the most frequent alleles HLA-A*02:01 as a representative, Applicants identified all conserved residues between the HC and hβ₂m within a Cβ-Cβ distance of 5.5 Å, and selected G120 and H31 for cysteine substitutions, this design was validated using Disulfide by Design (DbD) (Craig, D. B. & Dombkowski, A. A. Disulfide by Design 2.0: a web-based tool for disulfide engineering in proteins. BMC Bioinformatics 14, 346 (2013)), which predict pairs of residues likely forming a disulfide bond if mutated to cysteines in order to improve protein stability and assist in the study of protein dynamics.

Methods to produce soluble recombinant MHC molecules with which peptides of the invention can form a complex are known in the art. Suitable methods include, but are not limited to, expression and purification from E. co/i cells or insect cells. A suitable method is provided in the Example herein. Alternatively, MHC molecules may be produced synthetically, or using cell free systems. In an advantageous embodiment, for the generation of pMHC-I molecules, in vitro refolding may be performed by diluting a mixture of ds MHC-I and ds hβ₂m at a 1:3 molar ratio in refolding buffer. The mixture may be protected from light when refolded with photolabile peptides. Refolding may proceed over several days, and proteins may be purified by, for example, size exclusion chromatography (SEC).

Polypeptides and/or polypeptide-MHC complexes of the invention may be associated (covalently or otherwise) with a moiety capable of eliciting a therapeutic effect. Such a moiety may be a carrier protein which is known to be immunogenic. KLH (keyhole limpet hemocyanin) and bovine serum albumin are examples of suitable carrier proteins used in vaccine compositions. Alternatively, the peptides and/or peptide-MHC complexes of the invention may be associated with a fusion partner. Fusion partners may be used for detection purposes, or for attaching said peptide or MHC to a solid support, or for MHC oligomerization. The MHC complexes may incorporate a biotinylation site to which biotin can be added, for example, using the BirA enzyme (O'Callaghan et al., 1999 Jan. 1; 266(1):9-15). Other suitable fusion partners include, but are not limited to, fluorescent, or luminescent labels, radiolabels, nucleic acid probes and contrast reagents, antibodies, or enzymes that produce a detectable product. Detection methods may include flow cytometry, microscopy, electrophoresis or scintillation counting. Fusion partners may include cytokines, such as interleukin 2, interferon alpha, and granulocyte-macrophage colony-stimulating factor.

Peptide-MHC complexes of the invention may be provided in soluble form, or may be immobilized by attachment to a suitable solid support. Examples of solid supports include, but are not limited to, a bead, a membrane, sepharose, a magnetic bead, a plate, a tube, a column. Peptide-MHC complexes may be attached to an ELISA plate, a magnetic bead, or a surface plasmon resonance biosensor chip. Methods of attaching peptide-MHC complexes to a solid support are known to the skilled person, and include, for example, using an affinity binding pair, e.g., biotin and streptavidin, or antibodies and antigens. In a preferred embodiment peptide-MHC complexes are labelled with biotin and attached to streptavidin-coated surfaces.

Peptide-MHC complexes of the invention may be in multimeric form, for example, dimeric, or tetrameric, or pentameric, or octameric, or greater. Examples of suitable methods for the production of multimeric peptide MHC complexes are described in Greten et al., Clin. Diagn. Lab. Immunol. 2002 March; 9(2):216-20 and references therein. In general, peptide-MHC multimers may be produced using peptide-MHC tagged with a biotin residue and complexed through fluorescent labeled streptavidin. Alternatively, multimeric peptide-MHC complexes may be formed by using immunoglobulin as a molecular scaffold. In this system, the extracellular domains of MHC molecules are fused with the constant region of an immunoglobulin heavy chain separated by a short amino acid linker. Peptide-MHC multimers have also been produced using carrier molecules such as dextran (WO02072631). Multimeric peptide MHC complexes can be useful for improving the detection of binding moieties, such as T cell receptors, which bind said complex, because of avidity effects.

The polypeptides of the invention may be presented on the surface of a cell in complex with MHC. Thus, the invention also provides a cell presenting on its surface a complex of the invention. Such a cell may be a mammalian cell, preferably a cell of the immune system, and in particular a specialized antigen presenting cell such as a dendritic cell or a B cell. Other preferred cells include T2 cells (Hosken, et al., Science. 1990 Apr. 20; 248(4953):367-70). Cells presenting the polypeptide or complex of the invention may be isolated, preferably in the form of a population, or provided in a substantially pure form. Said cells may not naturally present the complex of the invention, or alternatively said cells may present the complex at a level higher than they would in nature. Such cells may be obtained by pulsing said cells with the polypeptide of the invention. Pulsing involves incubating the cells with the polypeptide for several hours using polypeptide concentrations typically ranging from 10-5 to 10-12 M. Said cells may additionally be transduced with HLA molecules, such as HLA-A*02 to further induce presentation of the peptide. Cells may be produced recombinantly. Cells presenting peptides of the invention may be used to isolate T cells and T cell receptors (TCRs) which are activated by, or bind to, said cells, as described in more detail below.

The invention provides a nucleic acid molecule comprising a nucleic acid sequence encoding any of the peptides or proteins of the invention. The nucleic acid may be cDNA. Such a nucleic acid molecule can be synthesized in accordance with methods known in the art. Due to the degeneracy of the genetic code, one of ordinary skill in the art will appreciate that nucleic acid molecules of different nucleotide sequence can encode the same amino acid sequence.

The invention provides a vector comprising a nucleic acid sequence according to the third aspect of the invention. The vector may include, in addition to a nucleic acid sequence encoding only a peptide of the invention, one or more additional nucleic acid sequences encoding one or more additional peptides. Such additional peptides may, once expressed, be fused to the N-terminus or the C-terminus of the peptide of the invention. In one embodiment, the vector includes a nucleic acid sequence encoding a peptide or protein tag such as, for example, a biotinylation site, a FLAG-tag, a MYC-tag, an HA-tag, a GST-tag, a Strep-tag or a poly-histidine tag.

Suitable vectors are known in the art as is vector construction, including the selection of promoters and other regulatory elements, such as enhancer elements. The vector utilized in the context of the present invention desirably comprises sequences appropriate for introduction into cells. For instance, the vector may be an expression vector, a vector in which the coding sequence of the polypeptide is under the control of its own cis-acting regulatory elements, a vector designed to facilitate gene integration or gene replacement in host cells, and the like.

A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-chain, single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 Å1, the contents of which are herein incorporated by reference in their entirety.

The present invention also comprises use of the complex which comprises, consists essentially of or consists of (a) one or more proteins of the dsMHC sequences of any one of Tables 1-6 and/or a sequence in FIG. 2 or FIG. 9A with a G120 mutation and/or a H31 mutation as well as one or more nucleic acid(s) encoding the same and (b) one or more peptides capable of eliciting an immune response as well as one or more nucleic acid(s) encoding the same as a therapeutic agent for treating cancer, either by triggering an immune response or in combination with another therapeutic agent which may be, for example, a toxic moiety for use in cell killing, or an immunostimulating agent such as an interleukin or a cytokine.

The term “cancer” according to the invention comprises leukemias, seminomas, melanomas, teratomas, lymphomas, neuroblastomas, glioblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer and lung cancer and the metastases thereof. Examples thereof are lung carcinomas, mamma carcinomas, prostate carcinomas, colon carcinomas, renal cell carcinomas, cervical carcinomas, or metastases of the cancer types or tumors described above. The term cancer according to the invention also comprises cancer metastases and relapse of cancer.

The term “autoimmunity” according to the invention relates a system of immune responses of an organism against its own healthy cells, tissues and other normal body constituents. Any disease resulting from this type of immune response is termed an “autoimmune disease”. Prominent examples include, but are not limited to, celiac disease, post-infectious IBS, diabetes mellitus type 1, Henoch-Schönlein purpura (HSP) sarcoidosis, systemic lupus erythematosus (SLE), Sjögren syndrome, eosinophilic granulomatosis with polyangiitis, Hashimoto's thyroiditis, Graves' disease, idiopathic thrombocytopenic purpura, Addison's disease, rheumatoid arthritis (RA), ankylosing spondylitis, polymyositis (PM), dermatomyositis (DM), Alopecia Areata and multiple sclerosis (MS).

The term “infectious disease” according to the invention relates to a transmissible disease or communicable disease, or an illness resulting from an invasion of tissues by pathogens, their multiplication, and the reaction of host tissues to the infectious agent and the toxins they produce. Infectious diseases are caused by infectious agents (pathogens) such as, but not limited to, bacteria (e.g., Mycobacterium tuberculosis, Staphylococcus aureus, Escherichia coli, Clostridium botulinum, and Salmonella spp.), viruses and related agents such as viroids. (e.g. HIV, Rhinovirus, Lyssaviruses such as Rabies virus, Ebolavirus and Severe acute respiratory syndrome coronavirus 2), fungi, further subclassified into: Ascomycota, including yeasts such as Candida (the most common fungal infection); filamentous fungi such as Aspergillus; Pneumocystis species; and dermatophytes, a group of organisms causing infection of skin and other superficial structures in humans, basidiomycota, including the human-pathogenic genus Cryptococcus, parasites, which are usually divided into: unicellular organisms (e.g. malaria, Toxoplasma, Babesia), macroparasites (worms or helminths) including nematodes such as parasitic roundworms and pinworms, tapeworms (cestodes), and flukes (trematodes, such as schistosomes), arthropods such as ticks, mites, fleas, and lice, can also cause human disease, which conceptually are similar to infections, but invasion of a human or animal body by these macroparasites is usually termed infestation, and prions (although they do not secrete toxins).

The term “peptide disease target” according to the invention relates to compositions such as, but not limited to, allotypes, peptides, epitopes, cell populations, ligands, receptors, or binders that interact, or affect the expression of, a target of interest, which may be specific to a desired disease. Peptide disease targets can include or excludes peptides or ligands such as, guanine, cytidine, guanosine, 5-aminoimidazole-4-carboxamide-1-O-D-ribofuranoside (AICAR), uridine 5′-monophosphate (UMP), shikimic acid, 3-dehydroshikimic acid, ruidine, L-kynurenine, venlafanxine, salicylic acid, salicyluric acid, guanosine-5′-monophosphate (GMP), adenosine 5′-monophosphate monohydrate (AMP), or cytidine 5′-monophosphate (CMP).

The therapeutically active agents, vaccines and compositions described herein may be administered via any conventional route, including by injection or infusion. The administration may be carried out, for example, orally, intravenously, intraperitoneally, intramuscularly, subcutaneously or transdermally. In one embodiment, administration is carried out intranodally such as by injection into a lymph node. Other forms of administration envision the in vitro transfection of antigen presenting cells such as dendritic cells with nucleic acids described herein followed by administration of the antigen presenting cells.

The agents described herein are administered in effective amounts. An “effective amount” refers to the amount which achieves a desired reaction or a desired effect alone or together with further doses. In the case of treatment of a particular disease or of a particular condition, the desired reaction preferably relates to inhibition of the course of the disease. This comprises slowing down the progress of the disease and, in particular, interrupting or reversing the progress of the disease. The desired reaction in a treatment of a disease or of a condition may also be delay of the onset or a prevention of the onset of said disease or said condition.

An effective amount of an agent described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the agents described herein may depend on various of such parameters. In the case that a reaction in a patient is insufficient with an initial dose, higher doses (or effectively higher doses achieved by a different, more localized route of administration) may be used.

The pharmaceutical compositions of the invention are preferably sterile and contain an effective amount of the therapeutically active substance to generate the desired reaction or the desired effect.

The pharmaceutical compositions of the invention are generally administered in pharmaceutically compatible amounts and in pharmaceutically compatible preparation. The term “pharmaceutically compatible” refers to a nontoxic material which does not interact with the action of the active component of the pharmaceutical composition. Preparations of this kind may usually contain salts, buffer substances, preservatives, carriers, supplementing immunity-enhancing substances such as adjuvants, e.g., CpG oligonucleotides, cytokines, chemokines, saponin, GM-CSF and/or RNA and, where appropriate, other therapeutically active compounds. When used in medicine, the salts should be pharmaceutically compatible. However, salts which are not pharmaceutically compatible may be used for preparing pharmaceutically compatible salts and are included in the invention. Pharmacologically and pharmaceutically compatible salts of this kind comprise in a non-limiting way those prepared from the following acids: hydrochloric, hydrobromic, sulfuric, nitric, phosphoric, maleic, acetic, salicylic, citric, formic, malonic, succinic acids, and the like. Pharmaceutically compatible salts may also be prepared as alkali metal salts or alkaline earth metal salts, such as sodium salts, potassium salts or calcium salts.

A pharmaceutical composition of the invention may comprise a pharmaceutically compatible carrier. The term “carrier” refers to an organic or inorganic component, of a natural or synthetic nature, in which the active component is combined in order to facilitate application. According to the invention, the term “pharmaceutically compatible carrier” includes one or more compatible solid or liquid fillers, diluents or encapsulating substances, which are suitable for administration to a patient. The components of the pharmaceutical composition of the invention are usually such that no interaction occurs which substantially impairs the desired pharmaceutical efficacy.

The pharmaceutical compositions of the invention may contain suitable buffer substances such as acetic acid in a salt, citric acid in a salt, boric acid in a salt and phosphoric acid in a salt.

The pharmaceutical compositions may, where appropriate, also contain suitable preservatives such as benzalkonium chloride, chlorobutanol, paraben and thimerosal.

The pharmaceutical compositions are usually provided in a uniform dosage form and may be prepared in a manner known per se. Pharmaceutical compositions of the invention may be in the form of capsules, tablets, lozenges, solutions, suspensions, syrups, elixirs or in the form of an emulsion, for example.

Compositions suitable for parenteral administration usually comprise a sterile aqueous or nonaqueous preparation of the active compound, which is preferably isotonic to the blood of the recipient. Examples of compatible carriers and solvents are Ringer solution and isotonic sodium chloride solution. In addition, usually sterile, fixed oils are used as solution or suspension medium.

The invention also encompasses a use of any one of the herein disclosed complexes, nucleic acids, vectors or pharmaceutical compositions to screen a panel of candidate epitopic peptides to identify relevant peptide disease targets. In one embodiment, the disease is cancer, autoimmunity or an infectious disease.

Although the present invention and its advantages have been described in detail, it should be understood that various changes, substitutions and alterations can be made herein without departing from the spirit and scope of the invention as defined in the appended claims.

The present invention will be further illustrated in the following Example which is given for illustration purposes only and are not intended to limit the invention in any way.

### EXAMPLES

### Example 1: Materials and Methods

Peptides. Peptide sequences are given as standard single-letter codes. Peptides and the Cancer Genome Atlas (TCGA) mutant epitope library purchased from Genscript, Piscataway, USA, at >90% purity. Photolabile and fluorophore-labeled peptides were purchased from Biopeptek Inc, Malvern, USA, or Genscript, Piscataway, USA, at >90% purity. Peptides were solubilized in distilled water or buffer and centrifuged at 14000 rpm for 15 minutes. The concentration of each peptide solution was measured and calculated using the respective absorbance and extinction coefficient at 205 nm wavelength.

Recombinant protein expression, refolding, and purification. HLA-A*02:01 (G120C) and HLA-A*24:02 (G120C) disulfide (ds) mutant heavy chain and hβ₂m (H31C) ds mutant light chain were transformed into Escherichia coli BL21(DE3) cells using pET plasmid under the control of a T7 promoter (New England Biolabs). Cells were grown and harvested in the Luria-Broth medium, and inclusion bodies were pelleted and purified as previously described¹. For the generation of pMHC-I molecules, in vitro refolding was performed by slowly diluting a 100 mg mixture of ds MHC-I and ds hβ₂m at a 1:3 molar ratio over 24 hours in refolding buffer (0.4 M L-Arginine-HCl, 100 mM Tris pH 8, 2 mM EDTA, 5 mM reduced L-glutathione, 0.5 mM oxidized L-glutathione) containing 10 mg of the peptide. The mixture was protected from light when refolded with photolabile peptides. Refolding proceeded for 4 days, and proteins were purified by size exclusion chromatography (SEC) using a HiLoad 16/600 Superdex 75 μg column at 1 mL/min with 150 mM NaCl, 20 mM Tris buffer, pH 8.0. Purified proteins were further confirmed in reduced and non-reduced conditions using sodium dodecyl sulfate-polyacrylamide (SDS-PAGE) gel electrophoresis.

pMHC-I tetramer preparation. WT or ds mutant pMHC-I with BirA Substrate Peptide (BSP, LHHILDAQKMVWNHR)-tagged hβ₂m were biotinylated using the BirA biotin-protein ligase bulk reaction kit (Avidity), according to the manufacturer's instructions. Biotinylated molecules were washed using Amicon Ultra centrifugal filter units with a 10 kDa membrane cut-off, and the level of biotinylation was evaluated by SDS-PAGE gel shift assay in the presence of excess streptavidin. Biotinylated pMHC-I/hβ₂m was prepared at a final concentration of 2 mg/mL. Streptavidin-PE (Agilent Technologies, Inc.) at 4:1 monomer/streptavidin molar ratio was added to pMHC-I/hβ₂m over 10-time intervals every 10 mins at RT in the dark. The resulting pMHC-I/hβ₂m tetramers can be stored at 4° C. for up to 4 weeks.

Differential Scanning Fluorimetry. Differential Scanning Fluorimetry (DSF) was used to assess the thermal stabilities of the WT and ds versions of pMHC-I molecules. 7 μM of placeholder peptide-loaded MHC-I molecules were incubated with the desired peptide at 1:10 pMHC-I/peptide molar ratio at room temperature (RT) overnight and then mixed with 10×SYPRO Orange dye in a buffer of 150 mM NaCl, 20 mM sodium phosphate pH 7.2 to a final volume of 20 μL. Samples were loaded into MicroAmp Optical 384 well plate and ran in triplicates. The experiment was performed on a QuantStudio™ 5 Real-Time PCR machine with excitation and emission wavelengths set to 470 nm and 569 nm. The thermal stabilities of pMHC complexes were compared by plotting the first derivative of each melting curve and extracting the peak as the melting temperature (Tₘ). The thermal stability was measured by gradually increasing temperature at a rate of 1° C. per minute between 25° C. and 95° C. Data analysis and fitting were performed in GraphPad Prism v9.

Fluorescence polarization. Kinetic association of fluorescently labeled peptides and various pMHC-I was monitored by FP. pMHC-I was combined with an optimized concentration of fluorescently labeled peptides in FP buffer (150 mM NaCl, 20 mM sodium phosphate, 0.05% Tween-20, pH 7.4) to achieve a polarization baseline between 0 and 50 mP, determined via serial dilution. _{TAMRA}KLFGYPVYV (40 nM) and _{TAMRA}KYNPIRTTF (6 nM) were added to WT or ds mutant HLA-A*02:01 and HLA-A*24:02. The pMHC-I concentration remained constant across experiments at 200 nM. Fluorescently labeled peptides were directly added to the well plate to avoid extended incubation and loss of data. Wells were loaded with 100 μL of reaction and triplicates for each condition were performed at room temperature. The kinetic association was monitored for 2-12 hours, and polarization measurements were recorded every 60-130 seconds. Excitation and emission values used to monitor the fluorescence of TAMRA-labeled peptides were 531 and 595 nm.

Raw parallel (In) and perpendicular emission intensities (I_{⊥}) were collected and converted to polarization (mP) values using the equation 1000*[(I_{II}−(G*I_{⊥}))/(I_{II}+(G*I_{⊥}))]. G-factors of 0.33 was optimized for TAMRA-labeled peptides in calculating the overall fluorescence polarization. The data analysis method² was adapted and data fitting was performed in GraphPad Prism v9.

Design disulfide-stabilized pMHC-I/hβ₂m. A structure-guided approach was utilized to design a disulfide-linked (ds) mutant of the human MHC-I (Human Leukocyte Antigen, HLA) by introducing G120C to the heavy chain (HC) and H31C to human β₂m. An alignment of all known high-resolution pMHC-I structures with distinct sequences (n=54) from a previously developed database HLA3DB was performed using ClustalOmega³ and processed using ESPript⁴. Conserved regions were identified across different HLA allotypes. Using one of the most frequent alleles HLA-A*02:01 as a representative, Applicants conserved all residue between the HC and hβ₂m within a Cβ-Cβ distance of 5.5 Å, and selected G120 and H31 for cysteine substitutions. This design was validated using Disulfide by Design (DbD)⁵, which predict pairs of residues likely forming a disulfide bond if mutated to cysteines in order to improve protein stability and assist in the study of protein dynamics. To confirm the universal application of G120C and H31C cross-linkage across MHC-Is, Applicants aligned the structures from the entire HLA3DB database (n=215) using PyMOL⁶ and calculated the distribution of the Cβ-Cβ distances between these two selected residues. Different HLA allotypes with a conserved motif of AY×G×D from residue 117 to 122 had an average distance of 4.25 Å (3.7 Å<Cβ_{C120}-Cβ_{C31}≥4.9 Å) between G120C and H31C, which is within the 5.5 Å constraints for disulfide cross-linkage^{7,8}. Additionally, the AY×G×D motif of the heavy chain and residues 30 to 36 of hβ₂m compose flexible loop regions, which further enhances the probability that the two cysteine residues form an approximately 90° dihedral angle necessary for disulfide bond formation⁹.

### Example 1 References

1. Li, H., Natarajan, K., Malchiodi, E. L., Margulies, D. H. & Mariuzza, R. A. Three-dimensional structure of H-2Dd complexed with an immunodominant peptide from human immunodeficiency virus envelope glycoprotein 120. Journal of Molecular Biology 283, 179-191 (1998).
2. Buchli, R. et al. Real-Time Measurement of in Vitro Peptide Binding to Soluble HLA-A*0201 by Fluorescence Polarization. Biochemistry 43, 14852-14863 (2004).
3. Sievers, F. et al. Fast, scalable generation of high-quality protein multiple sequence alignments using Clustal Omega. Mol Syst Biol 7, 539 (2011).
4. Robert, X. & Gouet, P. Deciphering key features in protein structures with the new ENDscript server. Nucleic Acids Research 42, W320-W324 (2014).
5. Craig, D. B. & Dombkowski, A. A. Disulfide by Design 2.0: a web-based tool for disulfide engineering in proteins. BMC Bioinformatics 14, 346 (2013).
6. Schrödinger, LLC. The PyMOL Molecular Graphics System, Version 1.8. (2015).
7. Liu, T. et al. Enhancing protein stability with extended disulfide bonds. Proceedings of the National Academy of Sciences 113, 5910-5915 (2016).
8. Dombkowski, A. A., Sultana, K. Z. & Craig, D. B. Protein disulfide engineering. FEBS Letters 588, 206-212 (2014).
9. Van Wart, H. E., Lewis, A., Scheraga, H. A. & Saeva, F. D. Disulfide Bond Dihedral Angles from Raman Spectroscopy. Proc. Natl. Acad. Sci. U.S.A. 70, 2619-2623 (1973).
10. Robinson, J. et al. IPD-IMGT/HLA Database. Nucleic Acids Research 48, D948-D955 (2020).

### Example 2: Universal, Open MHC-I for Rapid Ligand Loading and Enhanced Complex Stability Across Classical and Non-Classical Allotypes

The polymorphic nature and intrinsic instability of class I major histocompatibility complex (MHC-I) and MHC-like molecules loaded with suboptimal peptides, metabolites, or glycolipids presents a fundamental challenge for identifying disease-relevant antigens and antigen-specific T cell receptors (TCRs), dampening the development of autologous therapeutics. Here, Applicants leverage the allosterically coupled binding between the peptide and the light chain (β₂ microglobulin, β₂m) to the MHC-I heavy chain (HC) through an artificially introduced disulfide bond between two conserved residues on the HC and β₂m interface to generate conformationally stable MHC-I. The disulfide-engineered MHC-I molecules can afford properly conformed protein complexes of enhanced stability compared to the wild type when suboptimally loaded with low- to moderate-affinity peptides. Using solution NMR, Applicants find the extent of conformational dynamics and rearrangement exhibited for the variant, from the local β₂m interacting sites of the peptide binding groove to the long-range amino acids on the α₂₋₁ helix and α₃ domain. The interchain disulfide bond stabilizes empty or peptide-loaded MHC-I in a peptide-receptive, open conformation. Applicants further demonstrate that these open MHC-I molecules exhibit preferable peptide exchange properties across multiple human leucocyte antigen (HLA) allotypes, covering representatives from five distinct HLA-A and B supertypes, the oligomorphic HLA-Ib, and the nonclassical MHC-I-related proteins. Applicants' structural design, therefore, provides universal open MHC-I for rapid peptide exchange, enabling a range of approaches to screen antigenic epitope libraries and probe polyclonal TCR repertoire in the context of highly various HLA allotypes.

The proteins of the class I major histocompatibility complex (MHC-I) are essential components of adaptive immunity in all jawed vertebrates⁽¹⁾. They function by displaying a broad spectrum of self, aberrant, or foreign epitopic peptides, derived from the endogenous processing of cellular proteins, on the cell surface, thereby enabling immune surveillance by cytotoxic T lymphocytes (CTL) and Natural Killer (NK) cells⁽²⁾. Classical MHC-I molecules are comprised of a 8-15 amino acid peptide, an invariable light chain human β₂ microglobulin (β₂m), and a highly polymorphic heavy chain (HC) that contains three extracellular domains (α₁, α₂, and α₃)⁽³⁾. The expansion of MHC-I genes in humans (human leukocyte antigens, or HLAs) has resulted in more than 35,000 alleles with polymorphic residues located on the peptide binding groove, composed by the α₁ and α₂ domains and a n-sheet floor⁽⁴⁾. The polymorphic nature of HLA allotypes leads to a diversity of displayed peptide repertoires and interactions with molecular chaperones, other components of the antigen processing pathway, and T cell receptors (TCR), which ultimately define immune responses and disease susceptibility. Classical HLA-A and HLA-B allotypes can be further classified into 12 supertypes according to their various peptide binding specificity, determined by primary anchoring interactions with the peptide positions 2, 3, 5, and Ω^{(5, 6)}. Therefore, recombinant peptide-loaded MHC-I (pMHC-I) molecules are typically generated using in vitro refolding together with synthetic peptides⁽⁷⁾ as soluble monomers and can be further prepared as tetramers or multimers⁽⁸⁾. These reagents have been one of the most important tools for detecting, isolating, and stimulating CTLs, and screening, optimizing, and identifying immunodominant T cell epitopes for immunotherapy, diagnosis, and vaccine development⁽⁹⁾.

The assembly and peptide loading of nascent pMHC-I molecules occurs in the lumen of the endoplasmic reticulum and involves many molecular chaperones, including the peptide loading complex (PLC)-restricted tapasin and the PLC-independent homologous, transporter associated with antigen processing (TAP)-binding protein related (TAPBPR)^{(10, 11)}. The folding of the MHC-I HC and the formation of disulfide bonds in the α₂ and α₃ domains is assisted by calnexin and ERp57⁽¹²⁾. The HC then assembles with β₂m to generate an empty heterodimer that is highly unstable for most MHC-I alleles, which is stabilized by association with tapasin, ERp57, calreticulin, and TAP in the PLC⁽¹³⁾. Both chaperones tapasin and TAPBPR can facilitate the binding of high-affinity peptides to confer the stability and proper trafficking of MHC-I, which finally resides on the cell surface for hours to days⁽¹⁴⁾. Loading of high-affinity peptides induces a “closed” conformation of the α₂₋₁ helix via negative allosteric modulation between non-overlapping peptide binding sites and tapasin/TAPBPR binding sites to release the chaperones⁽¹⁵⁾. However, peptide loading and β₂m binding to the HC are positively allosterically coupled, together stabilizing the ternary complex⁽¹⁶⁾. The peptide loading process is initiated by a rate-limiting step of β₂m association with the HC, which yields HCs with enhanced peptide binding affinity in the nanomolar range^{(17, 18)}. Consequently, maintaining a stable tertiary structure of pMHC-I requires not only the selection of high-affinity peptides but also the proper function of β₂m as a conformational chaperone^{(16, 17)}. Though under sub-physiological temperatures, peptide-deficient MHC-I heterodimers have been reported express on the cell surface, empty MHC-I conformers without peptides are unstable^{(19, 20)} due to the cross-talk between peptide and β₂m binding interfaces. Therefore, this intrinsic property has led to non-specific exogenous peptide binding and irreversible denaturation, limiting its application as an off-the-shelf reagent for ligand screening and T cell detection.

A tremendous amount of work has been dedicated to the biophysical characterization and engineering of pMHC-I molecules⁽²¹⁾, with particular efforts being made to understand the molecular mechanisms of peptide loading and developing different methods for peptide exchange⁽²²⁾. Conditional ligands, bound to the MHC-I, can be cleaved by UV exposure or released by increasing the temperature to generate empty molecules in exchange for a rescuing peptide^{(21, 23-25)}. Dedicated MHC-I chaperone, TAPBPR and its orthologs have also been used to stabilize an array of different MHC-I allotypes in a peptide-receptive conformation with a preferred binding to HLA-A over HLA-B and HLA-C alleles to promote the exchange of low- to intermediate-affinity peptides for high-affinity peptides in a process known as “peptide-editing”⁽²⁶⁻³⁰⁾. In addition, molecular dynamics (MD) simulation combined with a tryptophan fluorescence assay has shown that empty MHC-I are not molten globules like previously reported⁽³¹⁾ but have varying degrees of structure in the α₁ and α₂ helices^{(32, 33)}. More recent works have also stabilized peptide-deficient molecules by introducing a cysteine mutation to the tyrosine and the alanine at position 84 and 139 between the α₁ and α₂ helices in the presence of dipeptides, restricting the highly flexible F pocket of the peptide binding groove to mimic the peptide-bound state for common alleles, such as HLA-A*02:01, HLA-A*24:02, and HLA-B*27:05⁽³⁴⁻³⁶⁾. Other works have sought to stabilize the pMHC-I complex by characterizing the interaction of mutant or orthologous β₂m. A chimeric functional study using human and murine β₂m variants (mβ₂m) bound to HLA-A*02:01 and H-2Db demonstrated that β₂m has a greater affinity for H-2Db than mβ₂m, resulting in increased overall stability due to a marked increase in polarity and the number of hydrogen bonds⁽³³⁾. Another mutational study identified a S55V mutant β₂m characterized by its capability to stabilize pMHC-I molecules to a greater extent than the wild type (WT), enhancing peptide binding and the ability of CD8+ T cell recognition⁽³⁷⁾. These studies, altogether, have emphasized the importance and the possibility of generating conformationally stable, peptide-receptive MHC-I heterodimers across different allotypes through protein engineering.

Given the importance of β₂m to the pMHC-I complex formation and overall stability, Applicants propose an alternative structure-guided approach to engineering conformationally stable, peptide-receptive MHC-I by introducing a disulfide bond between the heavy and the light chain, hereafter Applicants refer to this platform as open MHC-I. Applicants exploit the allosteric mechanisms governing the assembly of MHC-I complexes by locking pMHC-I proteins in an open, peptide-receptive state via the introduction of G120C and H31C on the flexible loop regions of the HC and β₂m. Applicants show that the interchain disulfide bond increases the thermostability of low- and moderate-affinity peptide-loaded molecules. Utilizing solution nuclear magnetic resonance (NMR) and hydrogen-deuterium exchange mass spectrometry (HDX-MS), Applicants find that a peptide-receptive, open conformation was prolonged for both peptide-loaded and -deficient molecules without heterodimer dissociation. Applicants further demonstrate that engineered open variants have improved peptide exchange efficiency and overall stability across five HLA-A and -B supertypes^{(38, 39)}, oligomorphic HLA-Ib alleles, HLA-E and -G, and MHC-like molecule MR1 C262S. Finally, Applicants demonstrate that open MHC-I molecules, specifically open HLA-A*02:01 are functionally competent in detecting target-specific cell populations, serving as a potential universal platform to expand peptide epitopes and detect targeted receptors across a highly diverse array of HLA allotypes.

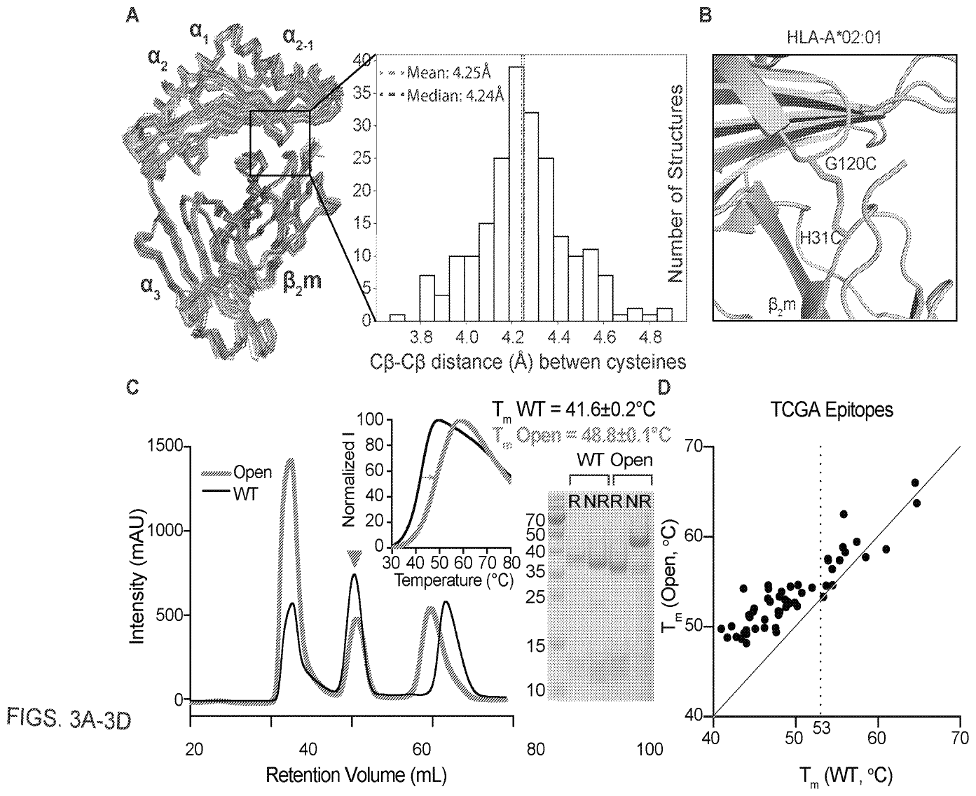

Structure-guided disulfide engineering stabilizes suboptimal peptide-loaded MHC-I. To engineer stable HLA molecules across different allotypes for rapid peptide exchange, Applicants aimed to bridge the HC and the light chain β₂m through a disulfide bond based on the positive cooperativity between peptide and β₂m association with the HCC^{(40, 41)}. Applicants utilized a structure-guided approach by first aligning 215 high-resolution pMHC-I crystal structures that were curated in Applicants' previously developed database, HLA3DB (FIG. 3A). Applicants found an average distance of 4.25 Å (3.7 Å≤Cβ-Cβ≤4.9 Å) between positions G120 of the HC and H31 of the β₂m (FIG. 3A). The distances between the paired residues G120 and H31 on the HC and the β₂m, respectively, fall within the molecular constraints (5.5 Å) for the disulfide cross-linkage^{(42, 43)}. The structure of HLA-A*02:01/β₂m shows that both regions are composed of flexible loops (FIG. 3B), which increase the probability of the two cysteine mutations forming a 90° dihedral angle necessary for the disulfide bond formation⁽⁴⁴⁾. Additionally, a sequence alignment using 75 distinct HLA allotypes with a greater than 1% global population frequency revealed a conserved glycine at position 120, suggesting a potential generality of the design across distinct HLA allotypes, covering various HLA supertypes that can present diverse peptide repertoires (FIG. 9). Selected residues G120 and H31 between the HC and β₂m were further computationally validated using Disulfide by Design⁽⁴⁵⁾. Together, the structure and sequence alignments indicate the possibility of applying the interchain disulfide cross-linkage to a broad range of HLA allotypes, including oligomorphic HLA-Ib and monomorphic nonclassical MHC-I-related proteins, to stabilize their ligand-receptive conformations.

Applicants next sought to validate the design experimentally by expressing the G120C variant of one of the most common alleles HLA-A*02:01 in Escherichia coli, isolated the denatured proteins from inclusion bodies, and folded it in vitro with the H31C variant of the β₂m in the presence of a low-affinity placeholder peptide TAX8 (LFGYPVYV). Size exclusion chromatography (SEC) and SDS/PAGE confirmed the formation of the G120C/H31C HLA-A*02:01/β₂m complex (hereafter referred to as “open HLA-A*02:01”) and the interchain disulfide bond (FIG. 3C). Applicants then performed differential scanning fluorimetry (DSF) and observed a substantial improvement in the thermal stability of the open HLA-A*02:01 compared to the WT with melting temperatures (Tₘ) of 48.8° C. and 41.6° C., respectively (FIG. 3C). Furthermore, the WT and open HLA-A*02:01/β₂m/TAX8 were further exchanged individually with 50 peptides from the Cancer Genome Atlas (TCGA) epitope library (TABLE 2). While the resulting Tₘ of the WT and open HLA-A*02:01 loaded with high-affinity peptides (WT Tₘ≥53° C.) were similar, a pronounced stabilizing effect was demonstrated on the open over WT HLA-A*02:01 when suboptimally loaded with low- to moderate-affinity peptides (WT Tₘ<53° C.) (FIG. 3D). Therefore, the scanning of thermal stabilities using HLA-A*02:01-restricted epitopes with a broad range of affinities showed that disulfide linkage between the HC and β₂m did not interfere with the peptide binding and consistently support the role of β₂m in chaperoning and stabilizing the HC for peptide loading.

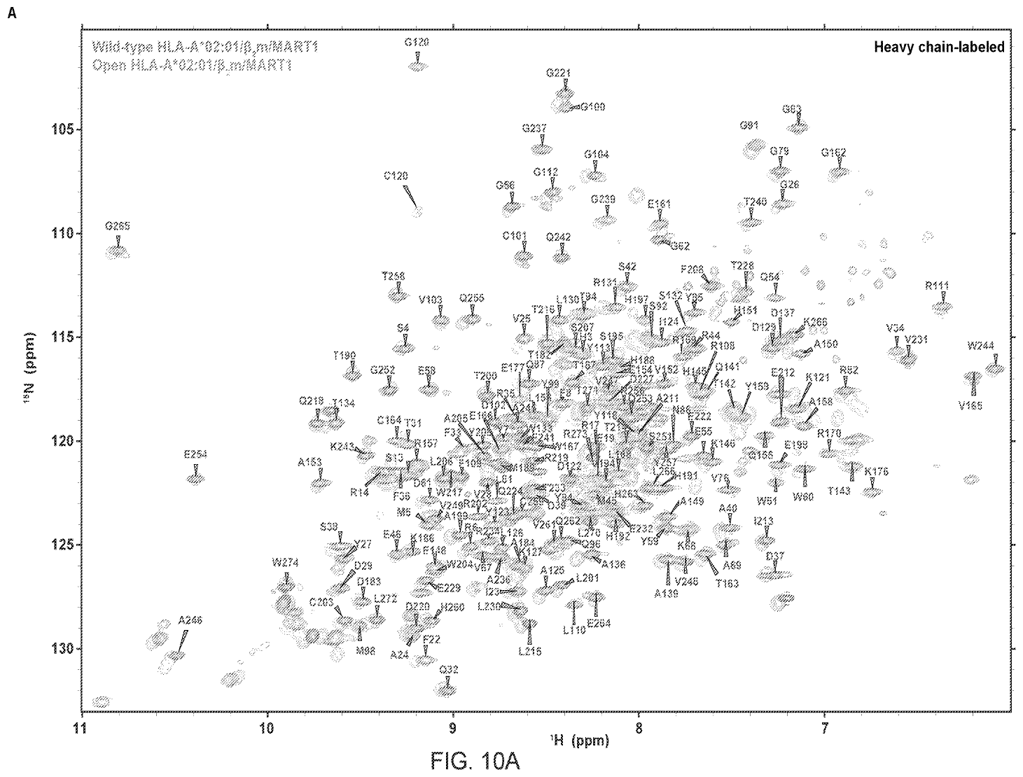

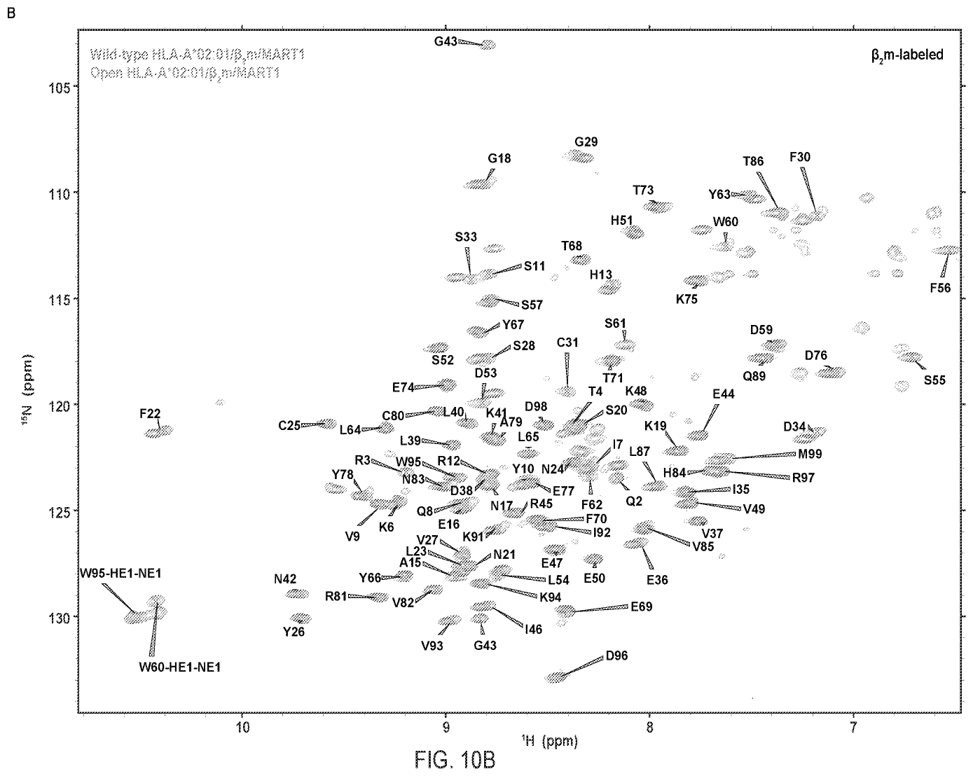

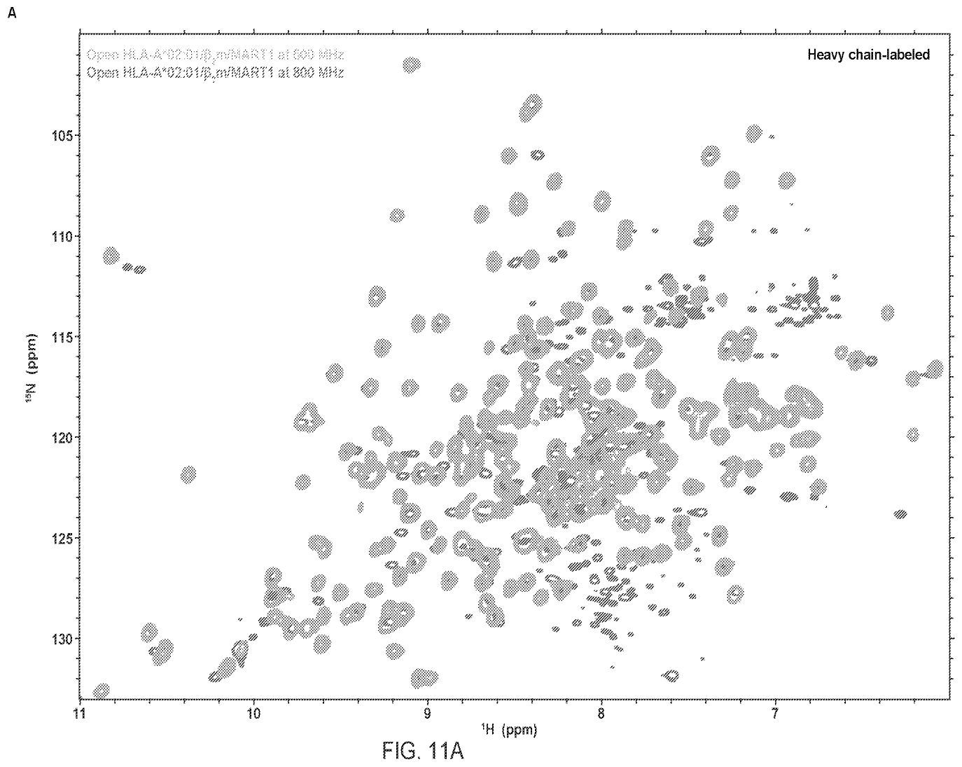

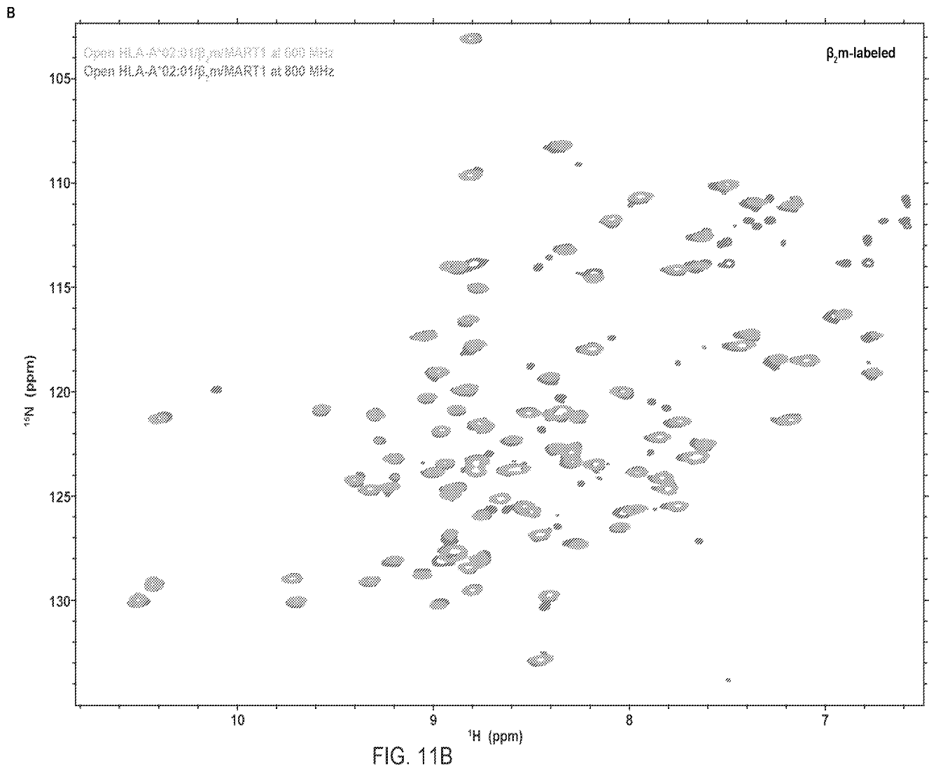

Disulfide-engineered pMHC-I exhibits local and long-range conformational dynamics. To elucidate the structural consequences of engineering the interchain disulfide bond for the peptide-loaded complex, Applicants utilized solution NMR to quantify the differences in the backbone chemical shifts between the WT and open HLA-A*02:01, defined as chemical shift perturbations (CSPs). Conformational dynamics have been previously implicated in several aspects of MHC-I function, including peptide loading, chaperone recognition, and TCR triggering⁽⁴⁶⁻⁴⁹⁾. Applicants first assigned backbone amides for both HC and β₂m across the WT and open HLA-A*02:01/β₂m/MART1 (ELAGIGILTV) (FIGS. 10A-11B). Applicants then calculated CSPs as a weighted measure of change in both ¹H and ¹⁵N dimensions of a 2D NMR spectrum, where residues with CSPs above the significant threshold of 0.05 ppm were mapped on the structure and indicated as experiencing altered chemical environments and structural rearrangements.

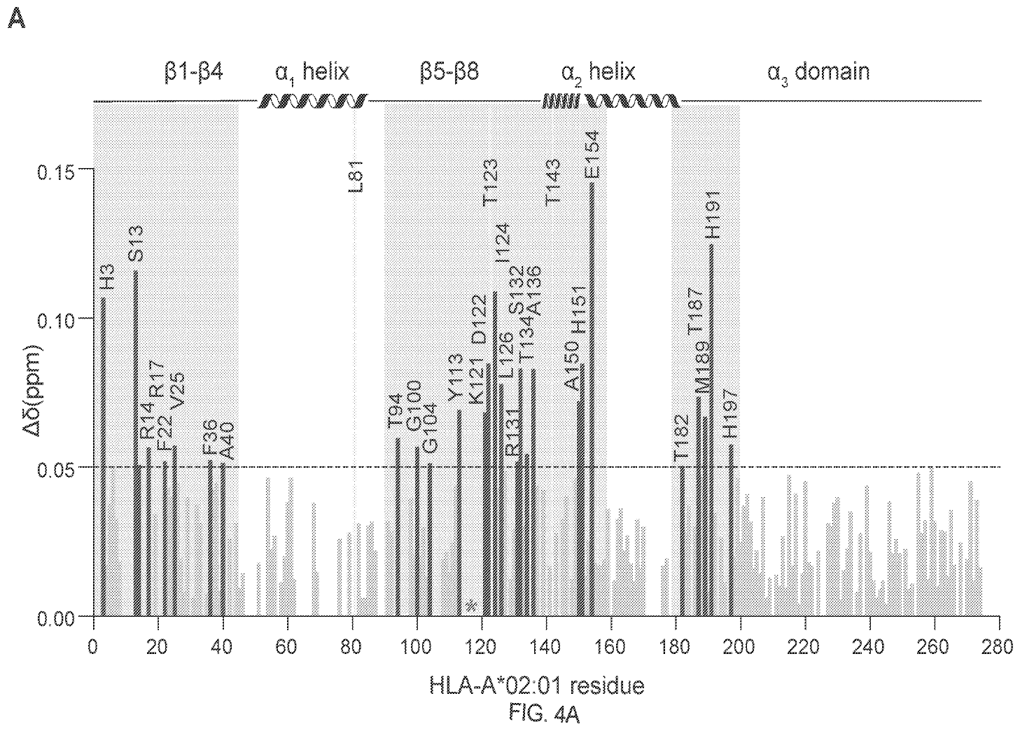

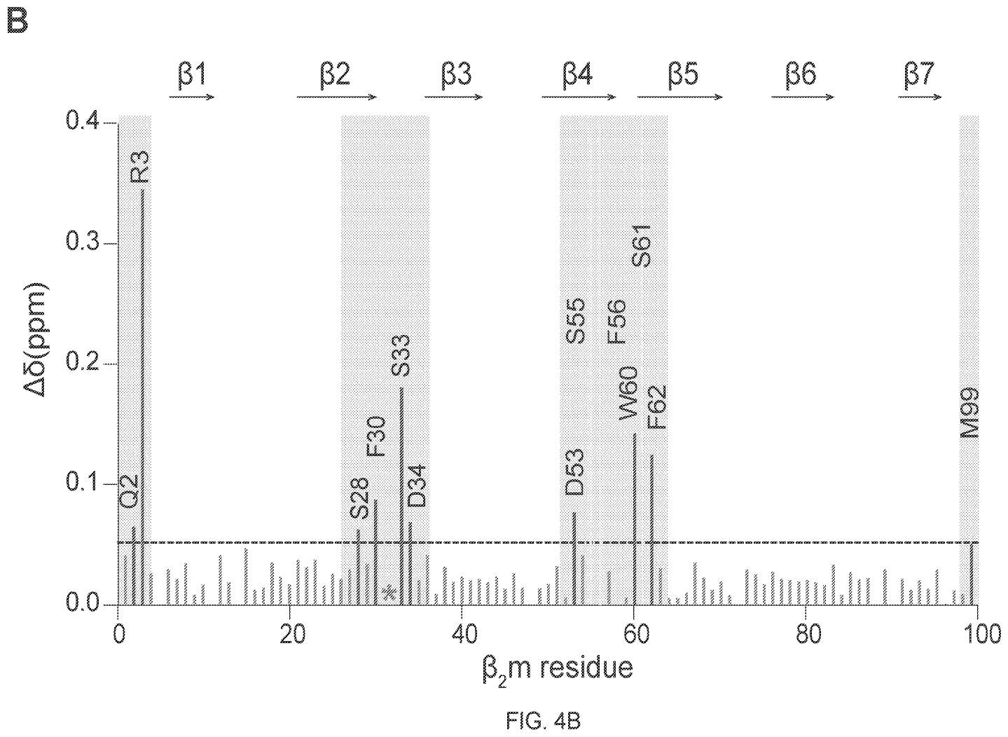

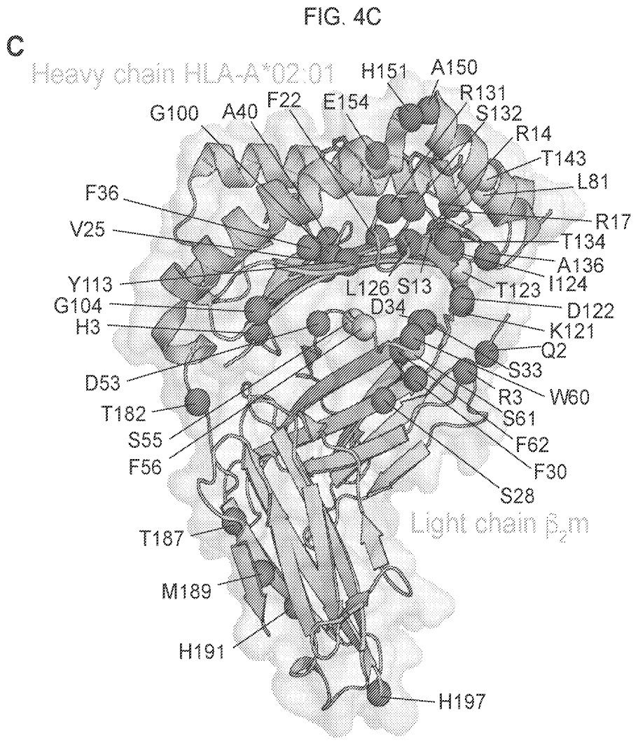

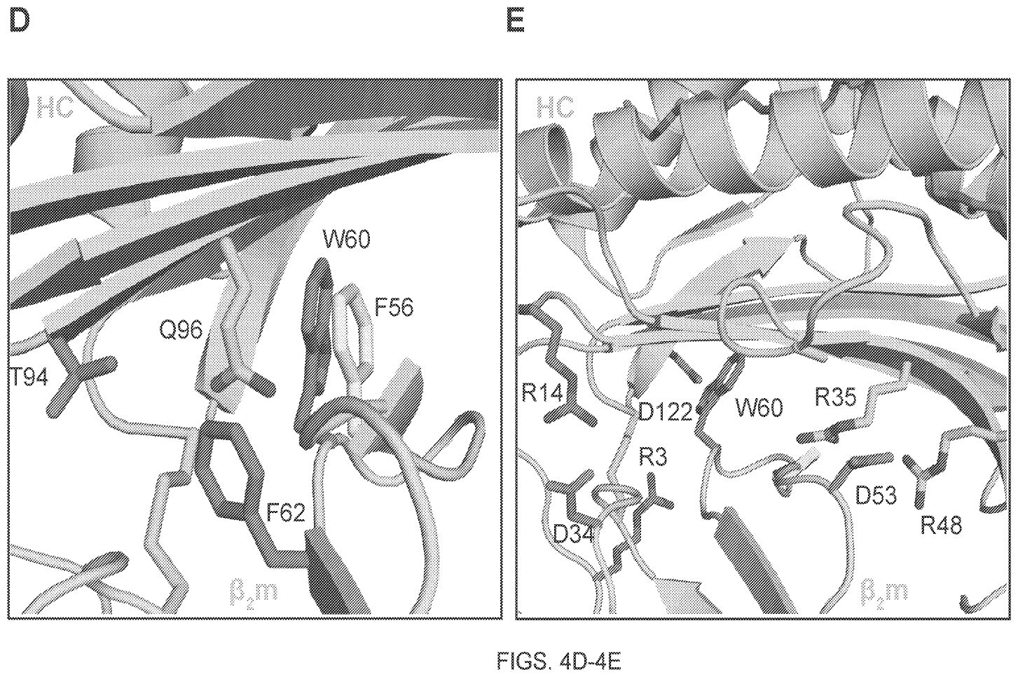

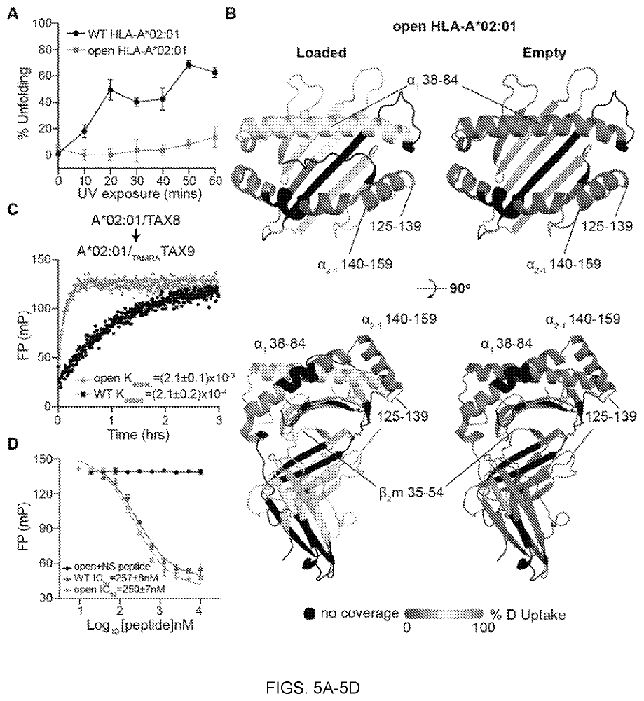

Applicants identified 38 residues that were affected by the formation of the interchain disulfide bond throughout the ternary complex, signifying substantial changes in conformational dynamics for the open relative to the WT molecules (FIGS. 4A-4B). As expected, most of the impacted residues were found near the HC and β₂m interface in the region surrounding the disulfide linkage (G120C and H31C) (FIG. 4C). Particularly, the β-sheet floor of the peptide binding groove, including the C, D, E, and F pockets were perturbed (FIGS. 4A, 4C). Arginine at position 3 located on the flexible loop region close to the engineered disulfide bond was the most affected on the β₂m (FIGS. 4B, 4C). Such a notable perturbation likely corresponds to the distinct motions of the nearby loops or the side chain rotamers, caused by the local structural rearrangement resulting from the disulfide linkage. Remarkably, Applicants' CSPs data indicated that residue W60 in β₂m underwent significant conformational changes and residue F56 displayed exchange broadening in the open but not in the WT, indicating completely altered microsecond to millisecond timescale dynamics (FIG. 5D). In agreement with the previous study, the specie-conserved phenylalanine residue at position 56 plays a central role at the interface between the β₂m subunit and the α₁α₂ domain of pMHC-I ^{(41, 50)}. Additionally, the allele-conserved residues F8, T10, Q96, and M98 in the HC and the residues like F56, W60, and F62 in β₂m together form the central part of a hydrophobic pocket(⁵¹). Therefore, the local perturbation observed for these residues upon covalently associating the HC and β₂m in close proximity might contribute to the overall stabilization of the suboptimal peptide-loaded MHC-I, given the known role of β₂m in the allosteric enhancement of peptide binding^{(52, 53)}.

Moreover, residues H31 and W60 in β₂m were also known to participate in the hydrogen bond interactions with residues Q96, G120, and D122 in the α₁α₂ domains⁽⁵¹⁾. Thus, H31C and G120C mutations influenced the nearby net hydrogen network, including R3, D34, D53, and W60 on hβ₂m and corresponding R14, R35, R48, and D122 (FIG. 5E). In addition, Applicants observed long-range CSPs resulting from the disulfide bond formation, on the α₂₋₁ helix and the far end of the β-sandwich fold on the α₃ domain (FIGS. 4A, 4C). This long-range effect along the α₂₋₁ helix of the peptide binding groove consistently supports that the interchain disulfide bond allosterically triggers conformational changes, where the α₂₋₁ helix has been previously demonstrated for its flexibility in the “open” and “closed” state of MHC-I for peptide loading. Similarly, the residues T182 located on the loop affected by the disulfide crosslinking might result in a structural repacking of T187, M189, H191, and H197 within the α₃ domain via a lever arm effect. Taken together, these results demonstrate that extensive local and long-range structural changes and dynamics arise from the interchain disulfide bond. Observing the allosteric effect on the α₂₋₁ helix allows us further to hypothesize that the engineered disulfide bond between the HC and β₂m might enhance peptide loading via a positive allostery conformational change of the peptide binding groove.

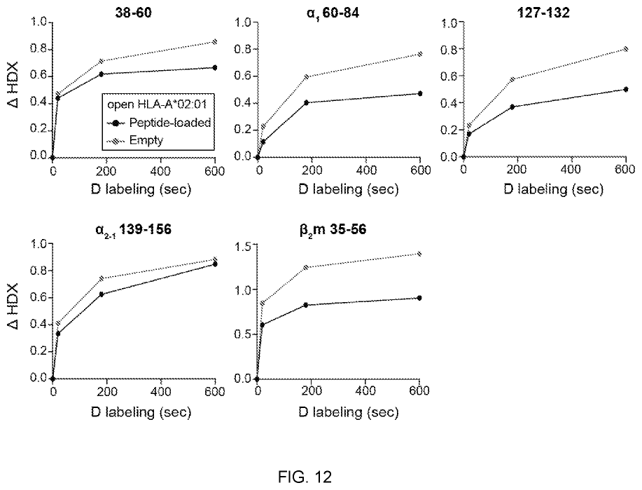

Interchain disulfide bond prolongs an open, receptive conformation of MHC-I. To test Applicants' hypothesis that the association between the β₂m and α₁α₂ interface could improve the overall stability and the peptide exchange capacity through an allosteric conformation change, Applicants first compared the percent unfolding of the WT and open HLA-A*02:01 refolded with a photo-sensitive peptide upon different periods of UV irradiation without a rescuing peptide. While the percent unfolding of the WT continuously increased, the open heterodimers showed no significant increase in the amount of unfolded protein (FIG. 5B). Applicants observed a 5-fold higher percent unfolding of the WT than the open variant upon 1-hour UV irradiation (FIG. 5B), consistent with Applicants' previous DSF results that the open HLA-A*02:01 is more stable when empty or loaded with low- to moderate-affinity peptides (FIG. 3D). Applicants next performed hydrogen-deuterium exchange-mass spectrometry (HDX-MS)⁽⁵⁴⁾ compared the local differences in packing between the peptide-loaded or empty open HLA-A*02:01. Tandem analysis of the percent deuterium uptake as a function of exchange reaction time revealed an exchange saturation of peptide fragments within 600 seconds (FIG. 12). The open HLA-A*02:01 in the empty state showed a major difference in HDX compared to the peptide-loaded state, including the peptide binding groove, the α₃ domain, and the β₂m (FIG. 5D). Low deuterium exchange on the regions corresponding to the ai helix and R-sheet floor of the peptide binding cleft for the loaded molecules were consistent with the results shown in the previous studies^{(30, 55)}. The α₂₋₁ helix was reported to have a higher deuterium uptake upon UV irradiation^{(30, 55)}, due to the widening of the groove by ˜3-Å observed for the empty “open” over peptide-loaded “closed” states^{(15, 56, 57)}. However, residues 140-159 on the open HLA-A*02:01 in the absence or presence of peptide showed no substantial difference in the percent deuterium uptake (FIG. 5B, FIG. 12). Such a similar, high-level deuterium exchange of the α₂₋₁ helix in both peptide-bound and empty states suggested that the interchain disulfide linkage might indeed stabilize the peptide binding groove in a prolonged “open” conformation, which could enhance the peptide receptivity of the MHC-I molecules.

To examine whether the stabilizing effect promotes peptide exchange of the open HLA-A*02:01 compared to the WT, Applicants compared the binding traces of a fluorophore-labeled peptide^{(58, 59)}. The observed apparent association rate constants (K_{assoc.}) were determined by fitting a one-phase association equation as the variable k in the exponential association equation was concentration dependent. Therefore, Applicants added the same concentration of the open HLA-A*02:01/TAX8 to the fluorescently labeled _{TAMRA}TAX9 compared to the WT, resulting in a K_{assoc.} that is 10-fold higher (FIG. 5C). The open HLA-A*02:01 exhibited a faster kinetics of peptide exchange. Accordingly, high-affinity TAX9 could be readily loaded into the open or WT HLA-A*02:01 molecules in a ˜200 nanomolar range, out competing the binding of fluorescent TAX9 (FIG. 5D). Despite different kinetics, TAX9 showed identical IC₅₀ blocking _{TAMRA}TAX9 association with the open or WT HLA-A*02:01(FIGS. 5C, 5D). Taken together, Applicants' data demonstrated that the engineered disulfide indeed allosterically prolonged an open conformation of the peptide binding groove and positively impacted the peptide exchange kinetics without noticeably influencing the IC₅₀ of the incoming peptide.

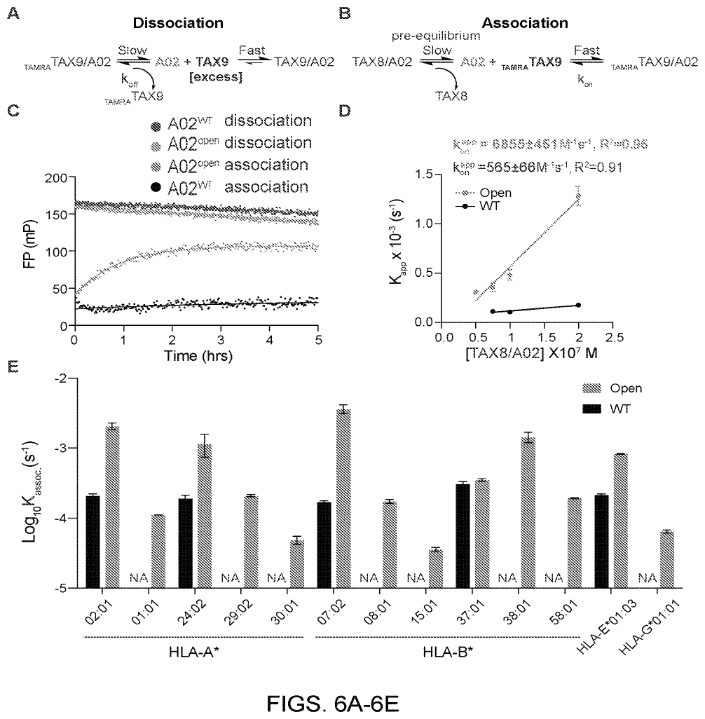

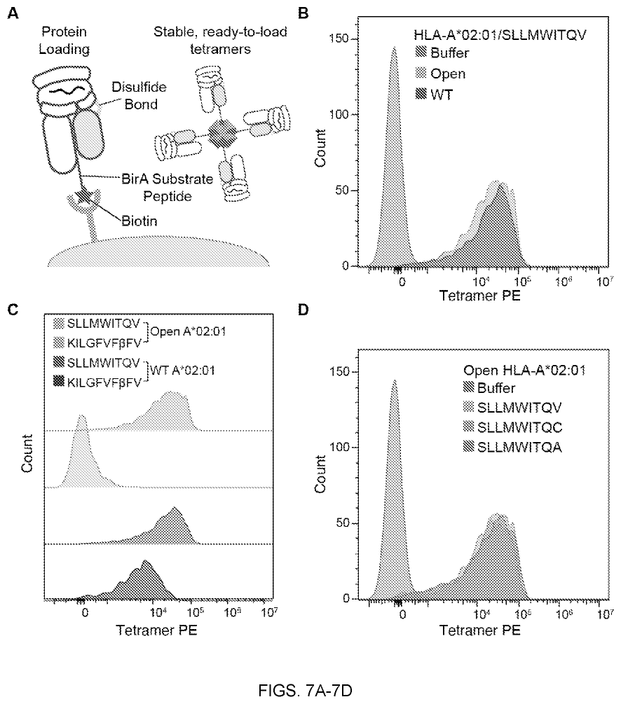

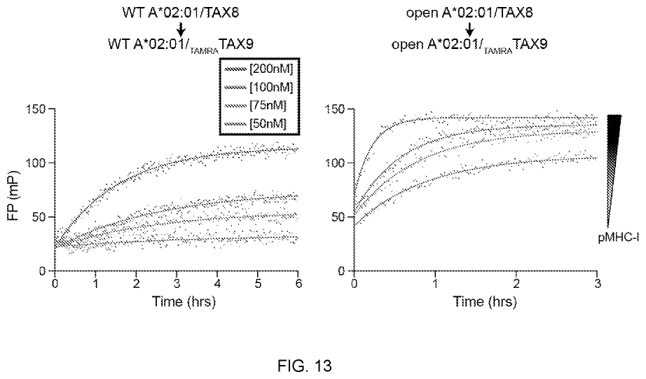

Open MHC-I demonstrates enhanced exchange efficiency on a broad repertoire of HLA allotypes. Applicants next sought to investigate how prolonging an open conformation could contribute to the rapid peptide exchange kinetics. Applicants studied both the dissociation and association of _{TAMRA}TAX9 (FIGS. 6A, 6B). When HLA-A*02:01 was refolded with a high-affinity peptide _{TAMRA}TAX9, the open molecules demonstrated a slightly faster decrease in polarization compared to the WT in molar excess unlabeled TAX9 (FIG. 6C). HLA-A*02:01-restricted strong binder _{TAMRA}TAX9 led to comparatively slow off-rates for both the open and WT molecules, which became the rate-limiting step in peptide dissociation (FIG. 6A). On the other hand, open HLA-A*02:01 molecules refolded with TAX8 mixed with molar equivalent _{TAMRA}TAX9 showed a substantial quicker peptide association and a higher plateau over the WT (FIG. 6C). The low-affinity placeholder-loaded TAX8/A02 compared to the TAX9/A02 demonstrated a more pronounced effect on peptide binding and resulted in more receptive molecules for the open variant than the WT at the same concentration (FIG. 13). Thus, Applicants could extrapolate an apparent kₒₙ defined as the slope of the linear correlation between K_{assoc.S} and the TAX8/A02 protein concentrations that attributed to the different amounts of empty, receptive molecules in the system (FIGS. 6B, 6D). Open HLA-A*02:01, compared to the WT, exhibited a more than 10-fold enhancement of the apparent kₒₙ, which was determined by both the formation rate of receptive molecules together with the stability of these empty molecules (FIG. 6D). Taken together, open HLA-A*02:01 demonstrated faster kinetics of peptide exchange because the rate-limiting, intermediate step of generating empty, receptive molecules is faster due to the allosteric effects on α₂₋₁ helix of the peptide binding groove induced by the β₂m association, as shown by NMR and HDX-MS.

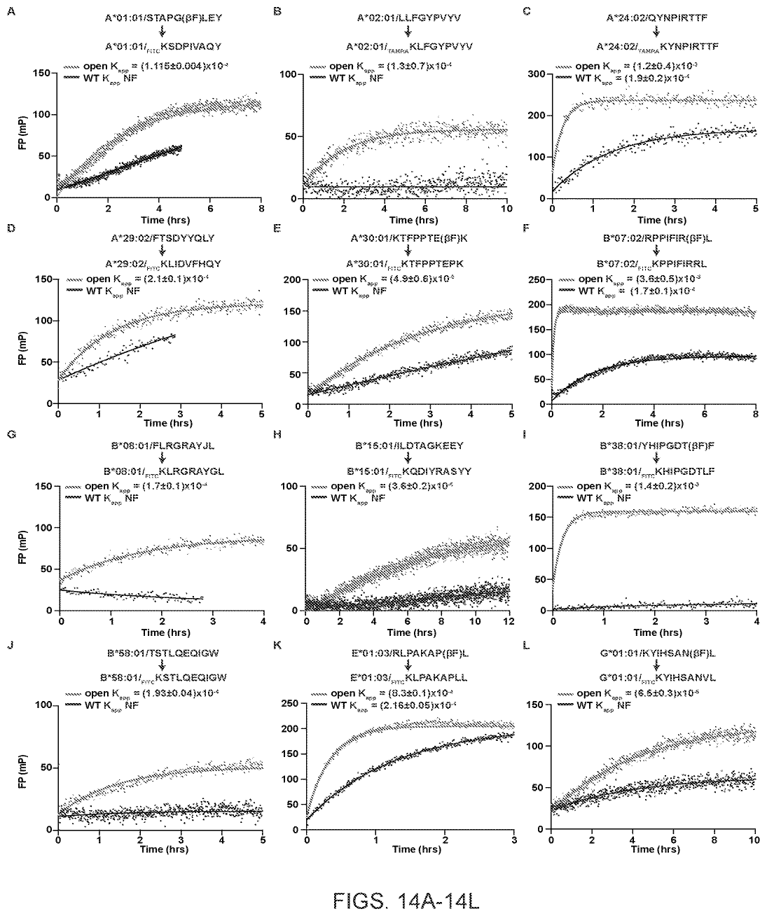

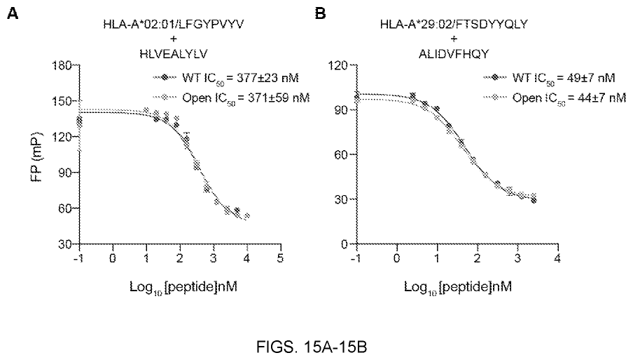

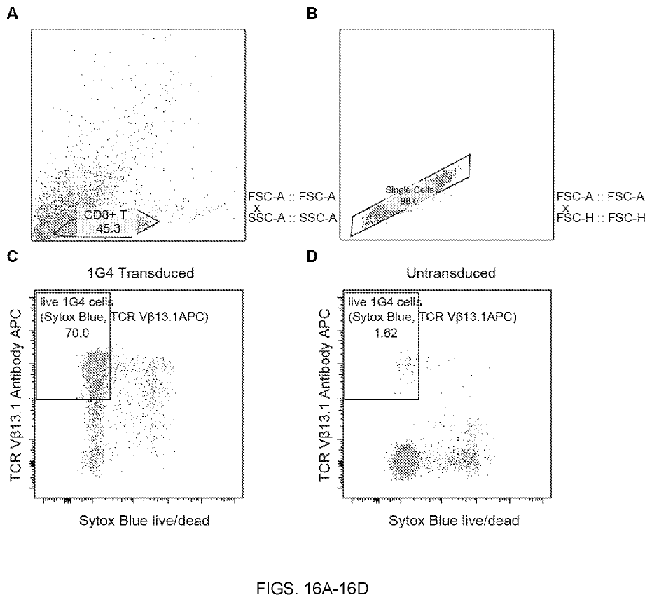

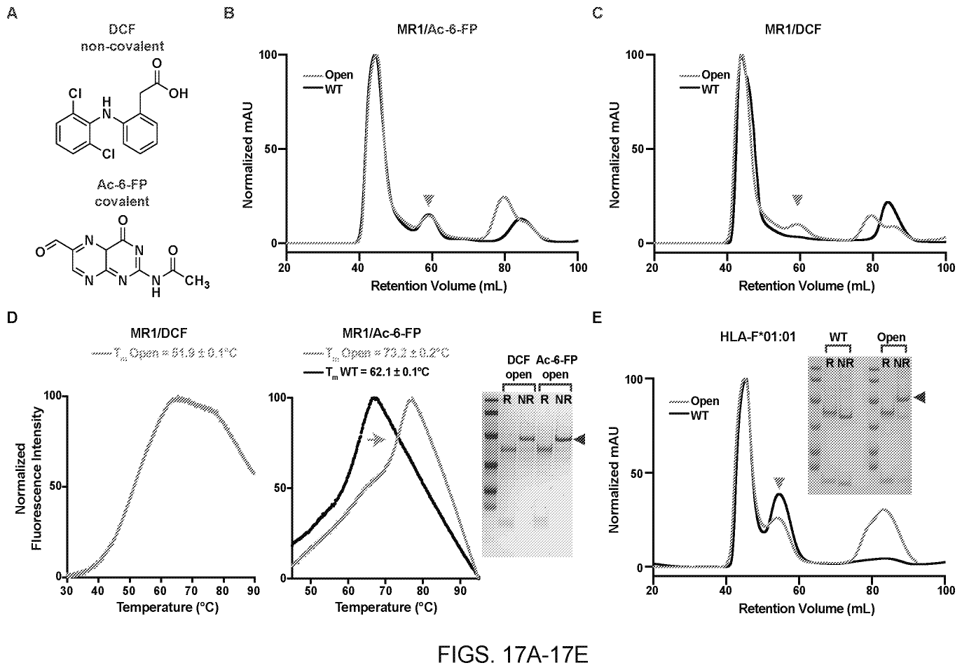

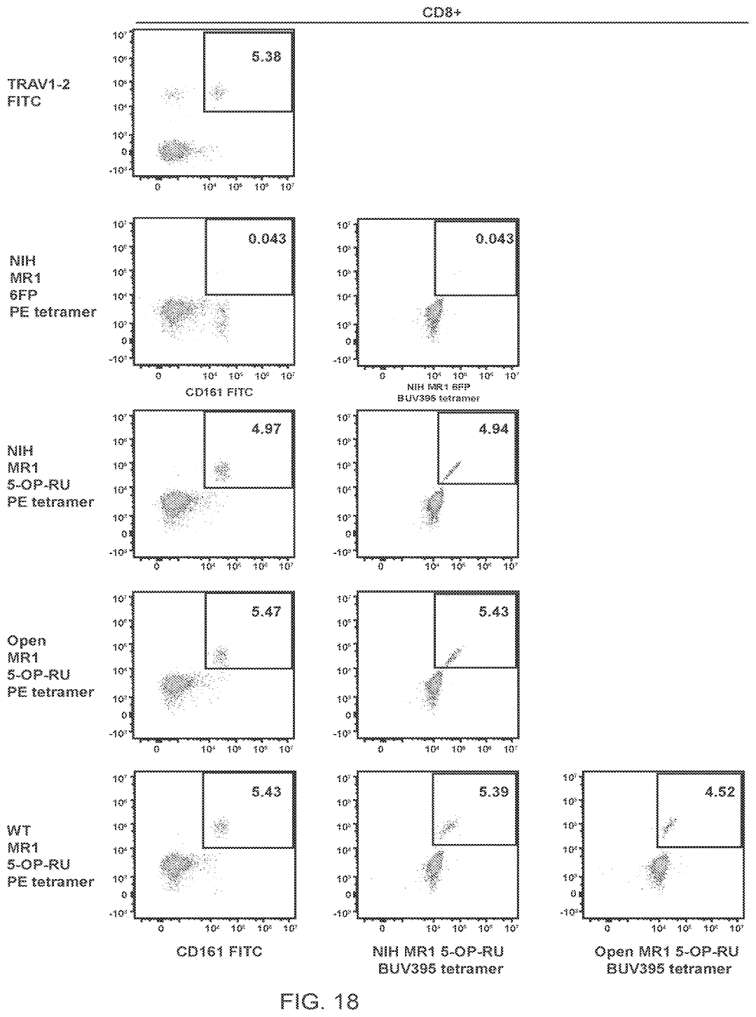

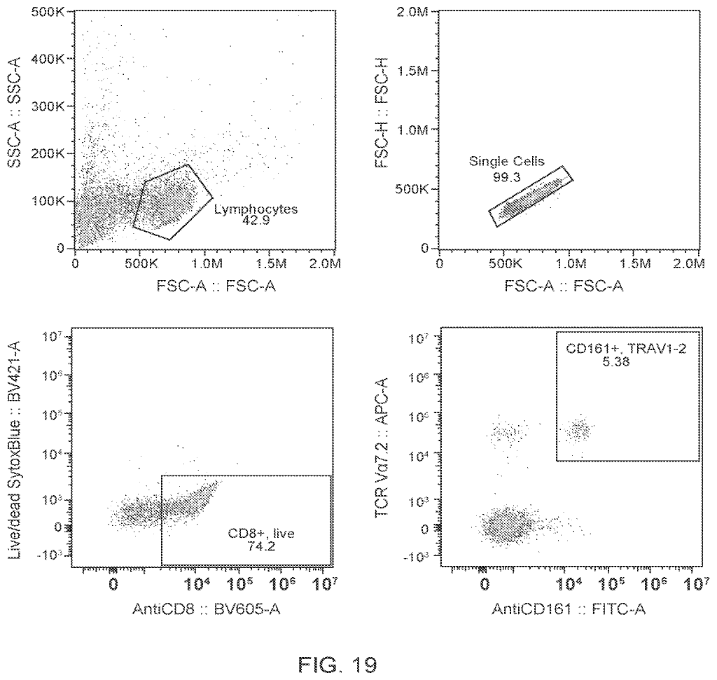

Applicants further hypothesized that the interchain disulfide engineering could be applied to different HLA allotypes resulting in universal, open MHC-I for rapid peptide loading. To quantitatively compare peptide exchange across different alleles, Applicants again performed a series of FP experiments using an optimal pHLA concentration and protocol previously described. The binding of high-affinity fluorophore-labeled peptides was traced through increasing polarizations in real time (FIGS. 14A-14L). Representatives covering five HLA-A and all HLA-B supertypes (A01, A02, A0103, A0124, A24 and B07, B08, B27, B44, B58, B62) were selected based on their global frequency (TABLE 3). Additionally, Applicants extended the study to cover the oligomorphic class Ib molecules, namely HLA-E*01:03 and HLA-G*01:01 (TABLE 3).

Applicants' FP results showed that open MHC-I (TABLE 4) demonstrated improved peptide exchange efficiency compared to the WT. Like open HLA-A*02:01 molecules, open HLA-B*07:02 exhibited a more than 20-fold increase in the apparent rate constant K_{assoc.} (FIG. 6E, FIG. 14E). Both open HLA-A*24:02 and HLA-E*01:03 displayed enhanced peptide exchange kinetics by approximately 6- and 4-fold (FIG. 6E, FIG. 14B, 14K). The remaining allotypes, HLA-A*01:01, A*29:02, A*30:01, B*08:01, B*15:01, B*38:01, B*58:01, and G*01:01, showed a fitted K_{assoc.} only in their open forms rather than in their WT counterparts, indicating no or slow exchange reaction (FIG. 6E, FIG. 14). Applicants consistently observed a stabilizing effect on low to moderate-affinity peptide-loaded molecules across alleles (WT Tₘ<53° C.) (TABLE 5). When loaded with a high-affinity peptide (WT Tₘ>53° C.), Tₘs generally stayed the same between the open and the WT except for HLA-G*01:01 (TABLE 5). Noticeably, suboptimally loaded HLA-B*37:01 in both open or WT format exhibited similar thermal stabilities and peptide exchange kinetics, revealing that receptive, empty molecules were pre-existing in the sample for peptide binding. Although open MHC-I demonstrated fast exchange kinetics, Applicants showed that two type 1 diabetes (T1D) epitopes, HLVEALYLV and ALIDVFHQY, has the same IC₅₀ towards both the WT and open variant for two different allotypes, HLA-A*02:01 and HLA-A*29:02. Altogether, Applicants demonstrated that a wide range of open HLA allotypes showed both enhanced thermal stabilities when loaded with suboptimal peptides and greatly accelerated peptide binding efficiency without losing their binding affinities in nanomolar range. These results provide additional evidence to support Applicants' hypothesis that the interchain disulfide bond locks MHC-I molecules in a stable, “open” conformation that readily loads peptides, resulting in a universal platform for ligand screening and T cell detection.

Application of open MHC-I as molecular probes for T cell detection and ligand screening. Applicants finally evaluated the use of open MHC-I in peptide exchange and tetramer-based T cell detection strategies. Applicants hypothesized that open molecules can be utilized as a universal off-the-shelf tool for screening peptide of interest to readily detect T cells with ultra-enhanced stability. To examine this hypothesis, Applicants conducted 1-hour RT peptide exchange of the same placeholder peptide-loaded HLA-A*02:01 in both WT and open form for a well-studied tumor epitope NY-ESO-1 (SLLMWITQV). BSP-tagged HLA-A*02:01 were purified, biotinylated, and tetramerized using streptavidin labeled with predefined fluorochromes (FIG. 7A). Applicants stained primary CD8+ T cells transduced with the TCR 1G4 that recognizes the NY-ESO-1 peptide presented by the HLA-A*02:01⁽⁶⁰⁾. Compared to the WT NY-ESO-1-loaded HLA-A*02:01, the open tetramer exhibited the same staining level (FIG. 7B). As negative controls, Applicants used both WT and open HLA-A*02:01 refolded with a non-specific placeholder peptide (KILGIVFβFV). Analysis by flow cytometry revealed a close-to-buffer background staining with the open HLA-A*02:01/KILGIVFβFV (FIG. 7C), suggesting that the 1G4 was unable to recognize the HLA-A*02:01/KILGIVFβFV. However, the WT HLA-A*02:01/KILGIVFβFV demonstrated a high level of background staining, exhibiting only one order of magnitude difference in intensity compared to the WT HLA-A*02:01/NY-ESO-1 (FIG. 7C). The noticeable difference in background staining might be caused by different amounts of empty molecules between the WT and open mutant. Empty HLA molecules can result in high background staining. The crosstalk between peptide and hβ₂m allows open HLA in preference to load peptide, avoiding the generation of empty molecules during the storage for tetramer/multimer reagents. Open HLA-A*01:01 also allowed moderate affinity peptides, SLLMWITQC and SLLMWITQA (NYESO C and A), to stain T cells consistently (FIG. 7D). Therefore, the functional staining indicates the possibility of using a high concentration of open HLA to identify rare and low-affinity TCRs without increasing background levels. In addition, elucidating the intrinsic selector function of different HLA allotypes through an engineered interchain disulfide has essential ramifications for developing barcoded libraries of pHLA antigens toward TCR repertoire characterization studies⁽⁶¹⁾.

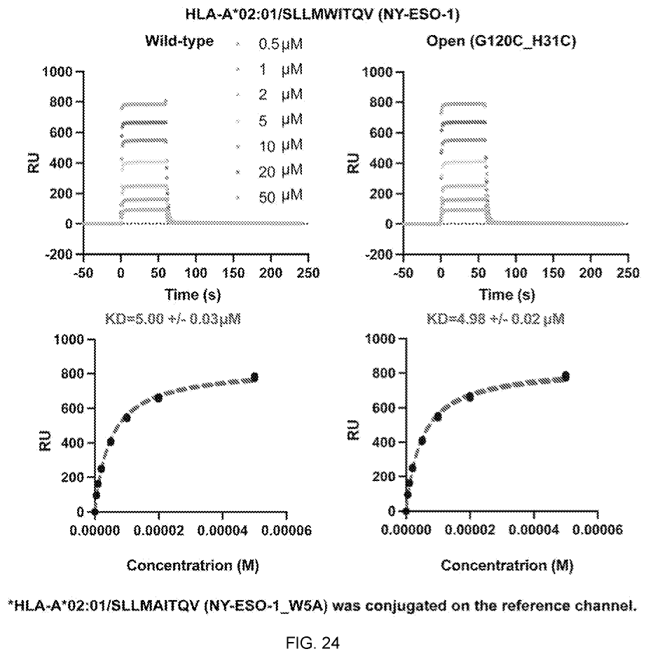

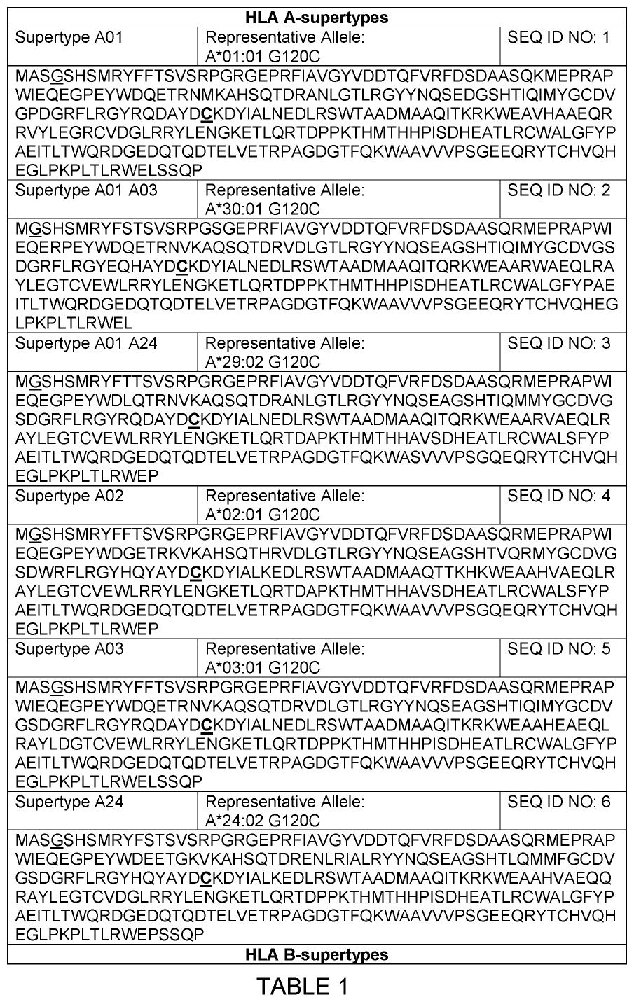

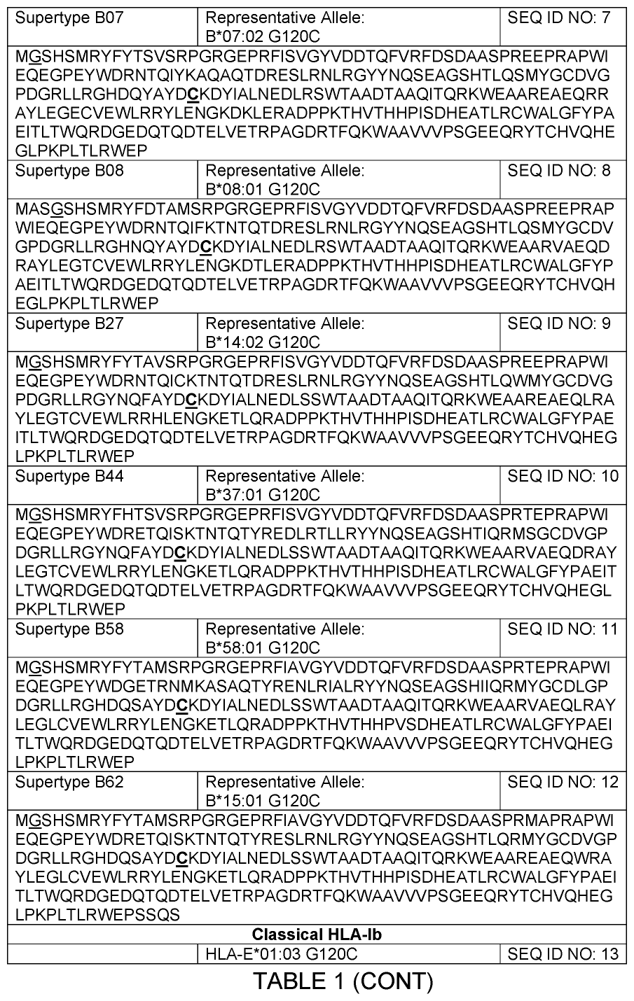

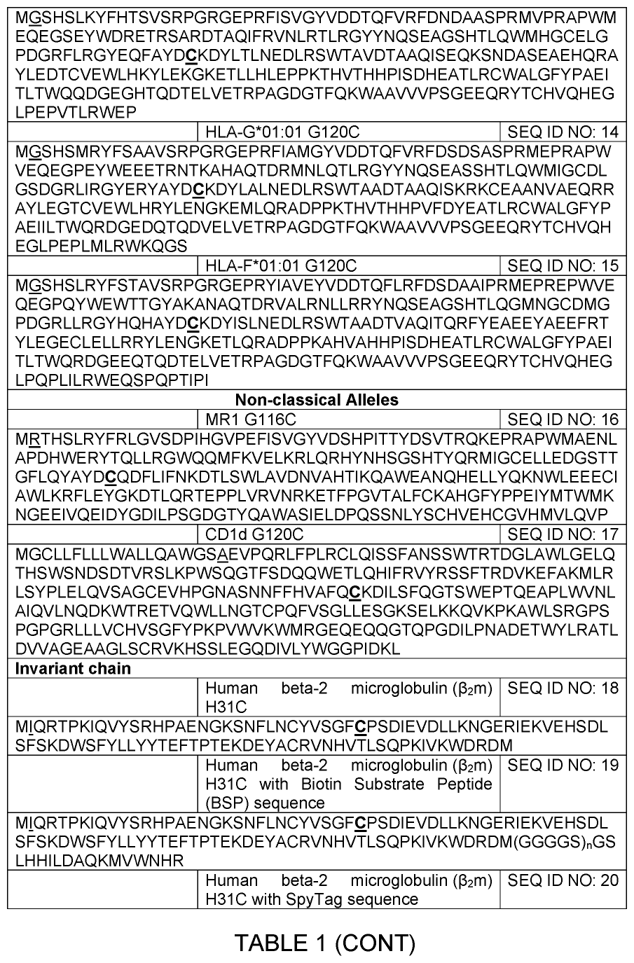

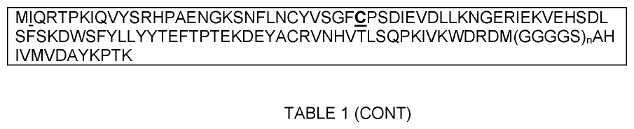

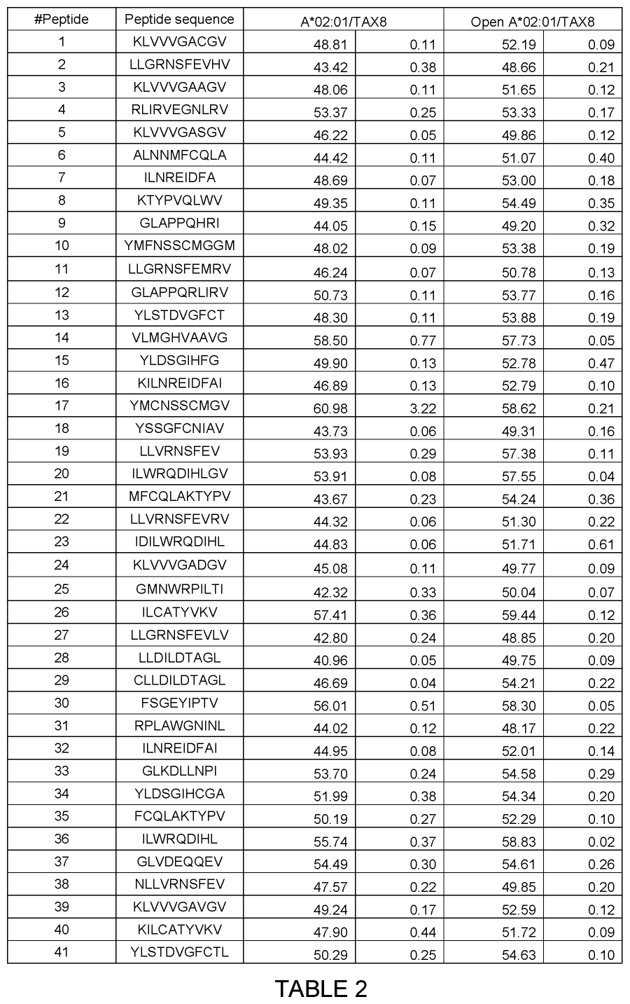

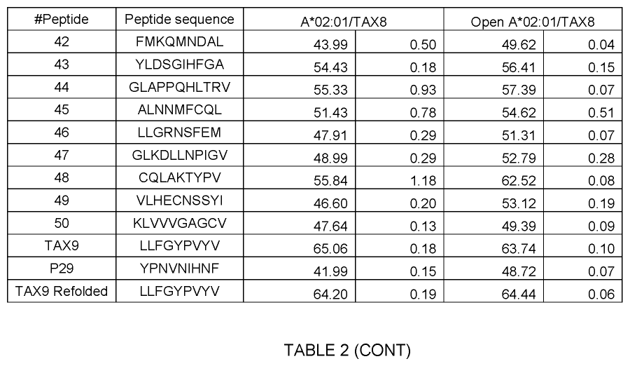

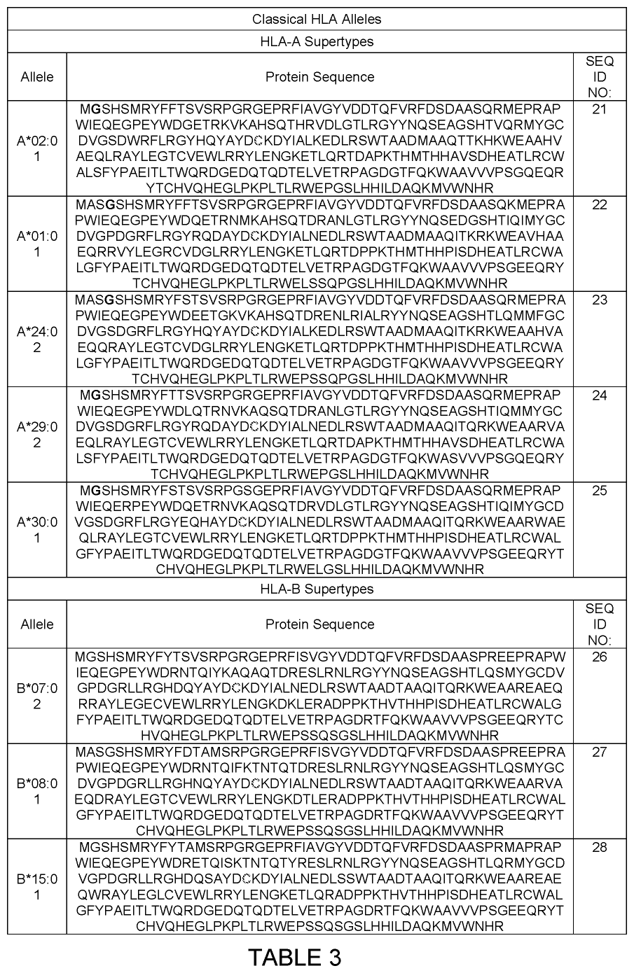

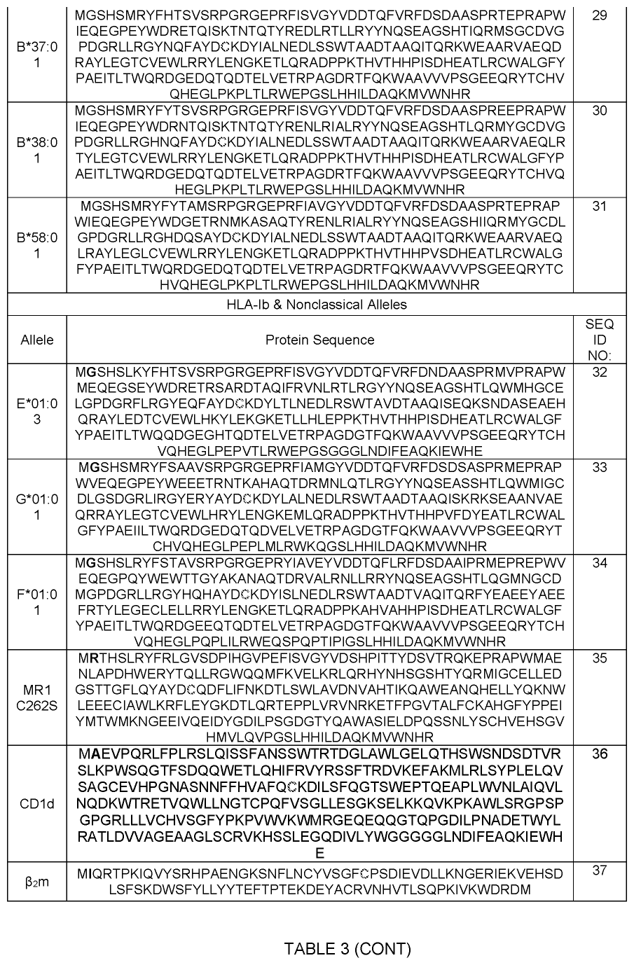

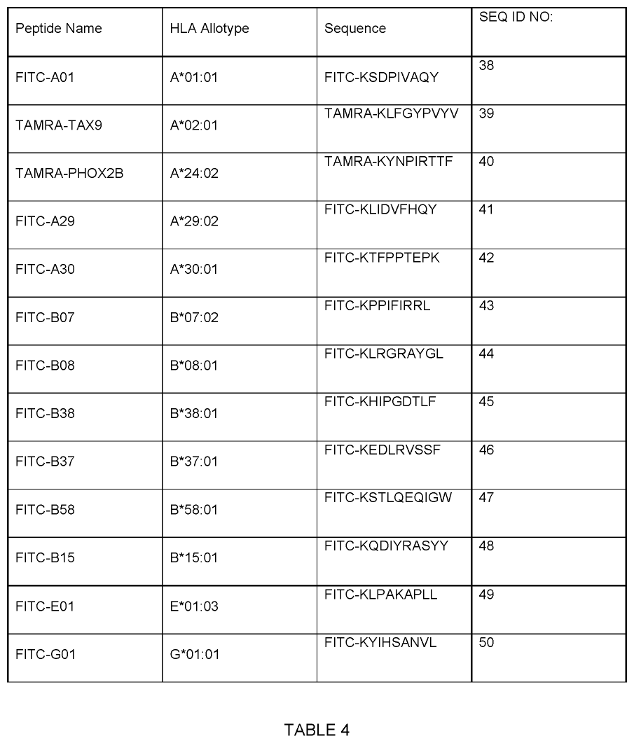

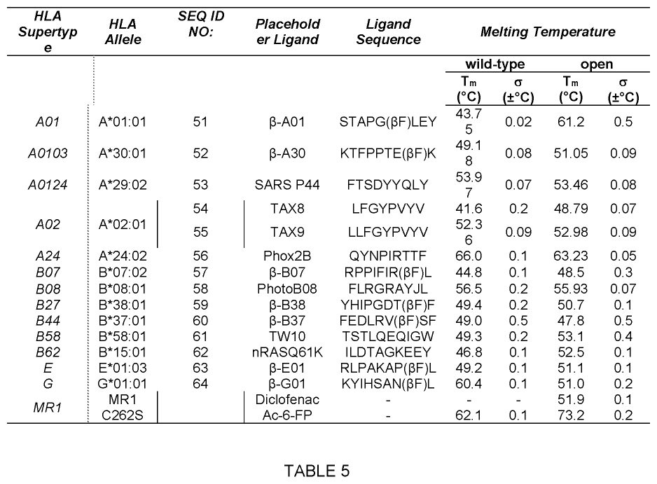

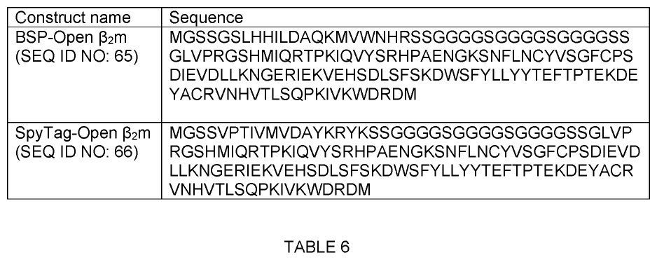

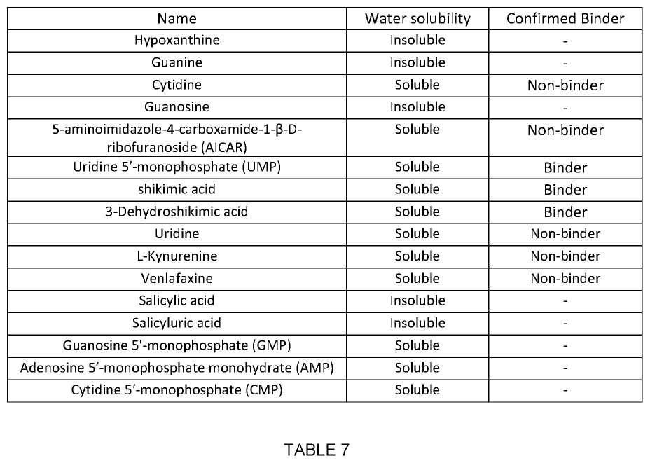

Applicants performed surface plasmon resonance (SPR) to determine whether the designed interchain disulfide bond influenced TCR binding affinity to HLA-A*02:01. As shown in FIG. 24, the designed interchain disulfide bond does not influence TCR binding affinity to HLA-A*02:01, exhibiting nearly the same equilibrium constant (KD).

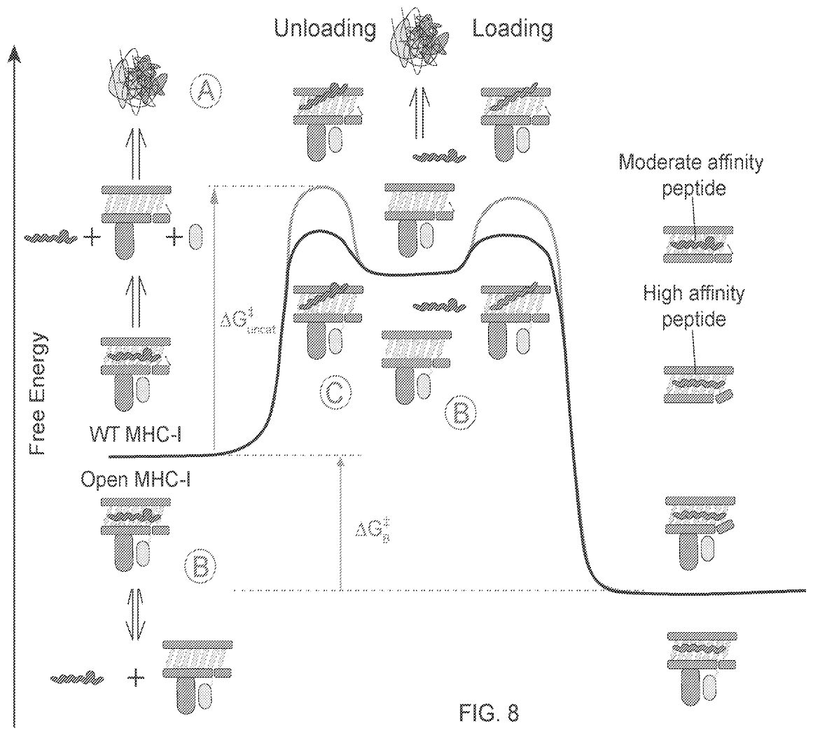

Applicants also extended the design of open HLA-I to non-classical MHC-I, MR1 and CD1, which can present small molecule metabolites and lipid antigens (FIGS. 8A-8B). Applicants first demonstrated that the open MR1 C262S construct loaded with a non-covalent small molecule DCF with a noticeable improvement of the protein yield (FIG. 8C). Applicants also observed a significant left shift of Tₘ of more than 10° C. for the open molecules MR1 C262S/Ac-6-FP and CD1d/α-GalCer compared to the WT (FIG. 8D, FIG. 14).

Applicants' combined biochemical and biophysical characterizations demonstrate a universal design of MHC-I molecules across different alleles suitable for ligand loading with enhanced stability. Compared to the UV- or heat-induced peptide exchange, open MHC-I mediated ligand exchange undergoes a mild exchange condition with 1 hour or overnight incubation at RT or 4C. Recombinant MHC-I molecules also undergo peptide exchange reactions with the addition of chaperones. While tapasin has shown preferential binding to HLA-B alleles in complex with other proteins belonging to the PLC, human TAPBPR preferably interacts with HLA-A alleles alone but mainly covers the A02 and A24 supertypes. Recent work has expanded the chaperone-mediated peptide exchange on a broader repertoire of alleles by using TAPBPR orthologs and engineered TAPBPR variants. Not only optimize the placeholder peptides and additive chaperones but efforts have also been made on MHC-I to stable, empty MHC-I molecules to load peptide epitopes and identify antigen-specific TLCs. The disulfide bond was first introduced by mutating the tyrosine and the alanine to cysteines at positions 84 and 139 between the α1 and α2 helices. While incorporating the disulfide bond on the F pocket of the peptide binding groove could stabilize the molecules in their empty form, it still requires dipeptide to initiate the in vitro refolding to bring the HC and hb2m in proximity, which was also tested in limited HLA alleles, such as HLA-A*02:01, HLA-A*24:02, and HLA-B*27:05. Recombinant open MHC-I utilize the positive cooperativity between the peptide association and hb2m binding to the HC to prolong the ligand-receptive conformation and allow the molecules undergo a spontaneous exchange. It demonstrates a comparable coverage of allotype representatives from 5 out of 6 HLA-A and HLA-B supertypes. Interchain disulfide bond brings minimal modification to allow spontaneous ligand exchange without interrupting the peptide binding and thermal stabilities of pMHC-I molecules and TCR interactions. Thus, engineered “open” MHC-I molecules open the chances to be applied as a valuable tool to screen and select ligand candidates, and detect and target low-frequent receptors and binders.

Applicants' structural and dynamic data altogether underline strong positive cooperativity between peptide binding and hβ₂m association with the HC. Applicants found different peptide exchange kinetics between the WT and the open HLA-A*02:01 loaded with the same placeholder peptide using fluorescence polarization. Both contained enough peptide-receptive molecules to accommodate the fluorescent peptides in the system. IC50s of the competitive peptide are similar between the WT and open molecules, indicating the difference in free energy of peptide binding stays the same. Incoming peptides exhibited the same affinity towards both the WT and open MHC-I regardless of the placeholder ligands, consistently suggesting an intermediate state of MHC-I that is empty and peptide-receptive. In this simplified ligand exchange reaction in vitro, the WT and open MHC-I loaded with a low- to moderate-affinity peptide can undergo peptide dissociation to generate empty molecules, which are naturally unstable in their heterodimer format waiting to be rescued by the peptide of interest. While the WT heterodimer will experience irreversible degradation to become protein aggregates for most of the allotypes, the open ones with the interchain cross-linkage form more stable empty molecules and allow equilibrium between peptide association and dissociation without substantial loss of receptive empty molecules for a longer duration. Therefore, hβ₂m linked covalently in proximity serves as a conformational chaperone to ensure a proper conformation of the HC and effectively avoid empty molecules undergoing irreversible aggregation.

Moreover, open molecules generally allow rapid peptide association by lowering the activation free energy for the transition of “open” and “closed” conformation. While the subsequent binding of incoming peptides requires empty MHC-I to transit from a “closed” to an “open” groove state, the loading of high-affinity peptides induced a “closed” groove conformation of the WT MHC-I again, which involves two activation energy barriers. However, Applicants' solution NMR and HDX-MS data demonstrated that the open MHC-I minimizes the transition process by allosterically influencing the alpha2-1 helix and extending the open conformation for both ligand-loaded and deficient molecules. Interchain disulfide bond further enhanced the affinity of the peptide binding groove towards incoming peptides via stabilizing the hydrophobic pocket and the hydrogen network between the HC and hb2m interface. Such a disulfide bond also introduces a rigidity of the conformationally open molecules, which allows enhanced thermal stabilities of pMHC-I even with rapid peptide exchange kinetics. Therefore, the same HLA allotype in the open format compared to the WT can exhibit a faster exchange kinetic when both contain sufficient receptive molecules. Taken together, Applicants' results showed that hb2m not only functions as a conformational chaperone to maintain the empty molecules in the system but lowers the activation energy of peptide unloading and loading by prolonging the open, receptive state.

A limitation of the present study is that Applicants did not fully distinguish the contribution of two main factors toward the enhanced peptide exchange kinetics of the open MHC-I. The subtle distinction of whether the increased number of empty molecules rescued by the interchain disulfide bond or the lowered activation free energy by the conformationally stable open molecules attributing the most is contemplated by Applicants and might require a dedicated study of thermodynamic parameters using Isothermal Titration Calorimetry (ITC) or high-resolution crystal structures of both the peptide-loaded and empty open HLA molecules. Furthermore, a follow-up study contemplated herein is using the open MHC-I to identify antigenic T cell epitopes and detect antigen-specific TLCs across different HLA allotypes was required to demonstrate the broad usage of this platform as off-the-shelf reagents. In summary, Applicants demonstrated the feasibility of an altered structure-guided design of conformational stable, peptide receptive MHC-I for rapid peptide exchange across five out of six HLA-A and -B supertypes, oligomorphic HLA-Ib alleles, HLA-E and -G, and nonclassical MHC-like molecules, MR1 and CD1d. The open MHC-I platform opens the opportunities for rapid ligand exchange for HLA allotypes like HLA-B07:02 that are not susceptible to chaperone-mediated peptide exchange and enhanced the protein yield and overall stability for alleles, such us HLA-E*01:03, MR1, and CD1d through in vitro refolding. Applicants confirmed the allosteric cooperativity between hb2m and peptide interactions with the HC exist and explained why the overall kinetics were enhanced on a molecular level using NMR and HDX. Such positively cooperative binding and structural understanding of the MHC-I can be applied to guide Applicants' design of the ultra-stabilized, universal ligand exchanger suitable in highly various allotype and antigen conditions.

Peptides and ligands. All peptide sequences are given as standard single-letter codes. Peptides for different HLA allotypes were selected by NetMHCpan4.1 and purchased from Genscript, Piscataway, USA, at >90% purity. L-β-Phenylalanine (βF) containing placeholder peptides were synthesized in-house on 2-chlorotrityl resin using a CEM Liberty Blue automated microwave peptide synthesizer from Fmoc protected amino acids (including Fmoc-p-Phe-OH) employing iterative cycles of N, N′-Diisopropylcarbodiimide (DIC)/Ethyl cyanohydroxyiminoacetate (Oxyma) mediated coupling and piperidine mediated deprotection, both under microwave irradiation. Peptides were deprotected and cleaved from the resin by treatment with trifluoroacetic acid/water/triisoproylsilane/phenol (88:5:5:2) for 1-3 hours. The solvent was removed under a flow of nitrogen, and peptides were precipitated with ice-cold ether. Peptides were subsequently purified by reverse phase chromatography eluting with 5-95% acetonitrile in water containing 0.05% trifluoroacetic acid over a C18 column. Peaks containing peptides were identified by LC-MS, pooled, and concentrated in vacuo to yield a colorless solid. Photolabile peptides were purchased from Biopeptek Inc, Malvern, USA, or synthesized in-house as above and using Fmoc-3-amino-3-(2-nitrophenyl)-propionic acid (J). Peptides were solubilized in distilled water and centrifuged at 14000 rpm for 15 minutes. The concentration of each peptide solution was measured and calculated using the respective absorbance and extinction coefficient at 205 nm wavelength. MR1 C262S ligand acetyl-6-formylpterin (Ac-6-FP) and diclofenac (DCF) were purchased from Cayman Chemical no. 23303 and Sigma D6899-10G. CD1d lipid α-Galactosyl Ceramide (α-GalCer) and sulfatides from bovine brain were purchased from Avanti Polar Lipids 867000P and Millipore Sigma S1006.

Recombinant protein expression, refolding, and purification. Plasmid DNA encoding the BirA Substrate Peptide (BSP, LHHILDAQKMVWNHR)-tagged luminal domain of MHC-I heavy chains and β₂m were provided by the NIH tetramer facility (Emory University) and transformed into Escherichia coli BL21 (DE3) cells (New England Biolabs). Open heavy chains (G120C) and β₂m (H31C) were generated using site-directed mutagenesis and transformed into Escherichia coli BL21(DE3) cells using pET plasmid under the control of a T7 promoter (New England Biolabs). Cells were grown and harvested in the Luria-Broth medium, and inclusion bodies were pelleted and purified as previously described⁽⁶²⁾. For the generation of pMHC-I molecules, in vitro refolding was performed by slowly diluting a 100 mg mixture of either wild-type (WT) or open MHC-I and β₂m at a 1:3 molar ratio over 4 days hours in refolding buffer (0.4 M L-Arginine HCl, 100 mM Tris pH 8, 2 mM EDTA, 5 mM reduced L-glutathione, 0.5 mM oxidized L-glutathione) supplemented with 10 mg of the peptide. The mixture was protected from light when refolded with photolabile peptides. Refolding proceeded for 4 days, and proteins were purified by size exclusion chromatography (SEC) using a HiLoad 16/600 Superdex 75 μg column at 1 mL/min with 150 mM NaCl, 20 mM Tris buffer, pH 8.0. Purified proteins were further confirmed in reduced and non-reduced conditions using sodium dodecyl sulfate-polyacrylamide (SDS-PAGE) gel electrophoresis. MR1 refolding was performed by diluting a 90 mg mixture of either WT or open 1:1.3 molar ratio overnight in refolding buffer supplemented with 5 mg of DCF or Ac-6-FP. Protein purification was performed as described above.

CD1d molecules were generated using an artificial chaperone mediated refolding method⁽⁶³⁾. A mixture containing 1 μM of CD1d heavy chain and 3 μM β₂m was diluted 70 folds with artificial chaperone refolding buffer (100 mM potassium phosphate buffer (pH 8.0), 0.1% Triton X-100, 1 mM EDTA, 0.3 M Arginine-HCl, 4 mM reduced L-glutathione, 0.4 mM oxidized L-glutathione) supplemented with 1 μM α-GalCer. The refolding reaction persisted for 4 days at 4° C. and detergent stripping buffer (16 mM β-cyclodextrin) was added in a 3:7 ratio to incubate overnight. The refolding mixture was dialyzed against running buffer (50 mM potassium phosphate buffer (pH 8.0), 150 mM KCl, 1% glycerol, and 60 mM guanidine hydrochloride) overnight and proteins were purified by size exclusion chromatography (SEC) using a HiLoad 16/600 Superdex 75 μg column at 1 mL/min with running buffer.

Differential Scanning Fluorimetry. Differential Scanning Fluorimetry (DSF) was used to assess the thermal stabilities of the WT and the open pMHC-I proteins. 7 μM of placeholder peptide-loaded MHC-I molecules were incubated with the desired peptide at 1:10 molar ratio at room temperature (RT) overnight and then mixed with 10×SYPRO Orange dye in a buffer of 150 mM NaCl, 20 mM sodium phosphate, pH 7.2 to a final volume of 20 μL. Samples were loaded into MicroAmp Optical 384 well plate and ran in triplicates. The experiment was performed on a QuantStudio™ 5 Real-Time PCR machine with excitation and emission wavelengths set to 470 nm and 569 nm. The temperature was incrementally increased at a rate of 1° C. per minute between 25° C. and 95° C. Data analysis and fitting were performed in GraphPad Prism v9.

To determine the percent unfolding, WT and open HLA-A*02:01 were UV irradiated for 0, 10, 20, 30, 40, 50, and 60 minutes. The full DSF traces were recorded at a constant rate of 1° C. per minute between 25° C. and 95° C. The fluorescence intensity (I) at 25° C. was then normalized against the maximum I. Data analysis and fitting were performed in GraphPad Prism v9.

NMR samples and methyl resonance assignment. NMR samples of WT and open HLA-A*02:01/β₂m/MART1 complex were prepared with an [¹⁵N, ¹²C, ²H] isotope-selective labeling scheme using established protocols and reagents^{(64, 65)}. Samples were in the concentration range of 50 to 150 μM prepared in a standard NMR buffer (150 mM NaCl, 20 mM sodium phosphate pH 7.2, 0.001 M sodium azide, 5% D₂O). The heavy chain and the light chain β₂m were isotopically labeled individually using M9 media in E. coli13 and refolded with natural isotopic abundance complex components, as described previously for the same system^{(15, 49)}. Backbone and methyl resonance assignments for the WT complexes were derived using a series of TROSY-based 2D and 3D experiments recorded at a 1H field of 600 or 800 MHz at 25° C., following a multi-pronged approach described previously for a similar system⁽⁶⁶⁾. Assignments were then transferred to the spectra of the open complexes and confirmed by TROSY-readout triple-resonance experiments (HNCO, HNCA, and HN(CA)CB), recorded at 600 MHz. Final backbone assignments were verified using the TALOS-N server⁽⁶⁷⁾. For chemical shift perturbation calculations, the WT and the open TROSY NMR spectra were aligned to each other using a residue at the maximum distance from the mutation sites as a reference in an existing crystal structure (HLA-A*02:01_E254 and β₂m D96; PDB ID: 3mrq). Amide backbone chemical shift perturbations between the WT and the open variant were calculated using the following equation, given the aligned ¹⁵N and ¹H chemical shifts:

$$Δδ ( ppm ) = \sqrt{{( {Δδ}_{H} )}^{2} + {( \frac{{Δδ}_{N}}{10} )}^{2}} .$$

All NMR data were processed with NMRPipe and analyzed using NMRFAM-SPARKYC^{(68, 69)}.

Hydrogen/Deuterium exchange mass spectrometry. The open HLA-A*02:01/KILGFVFJV was dialyzed into equilibration buffer (150 mM NaCl, 20 mM sodium phosphate, pH 6.5 in H₂O) and diluted to a stock concentration of 30 μM and then either i) kept on ice without exposure to UV light or ii) UV-exposed for 45 min at 4° C.

Samples were prepared and injected manually for several deuterium-exchange incubation periods. 5 μL open HLA-A*02:01/KILGFVFJV (30 μM) with or without UV-irradiation were diluted with 20 μL equilibration buffer (all H experiments, 0s) or deuterium buffer (150 mM NaCl, 20 mM sodium phosphate pD 6.5 in D₂O) to 6 μM. The proteins were incubated with deuterium buffer for 20, 180, and 600 seconds at RT, and 15 minutes at 43° C. for HLA-A*02:01/KILGFVFJV or at 34° C. for UV-irradiated HLA-A*02:01/KILGFVFJV as all the D samples to calculate ΔMass_{100%}. The samples were then quenched with an equal volume of acidic buffer (150 mM NaCl, 1 M TCEP, 20 mM sodium phosphate pH 2.35 in H₂O, 25 μL). Quenched proteins were immediately injected for LC-MS/MS in which integrated pepsin digestion was performed using a C8 5 μM column and a Q Exactive Orbitrap Mass Spectrometer. Peptide fragments corresponding to HLA-HLA-A*02:01 and β₂m were identified using Thermo Proteome Discoverer v2.4.

The percent deuterium uptake was back-exchange corrected for each time point using the following equation⁽⁵⁴⁾:

$$\% D = \frac{{ΔMass}_{T} - {ΔMass}_{0 \%} )}{{ΔMass}_{100 \%} - {ΔMass}_{0 \%} )} .$$

ExMs2 program was used to identify and analyze deuterated peptides. The kinetic plots and the scaled B factor for the structure plot were generated by python3 and PyMOL⁽⁷⁰⁾.

Fluorescence polarization. The kinetic association of fluorescently labeled peptides and various peptide-loaded MHC-I was monitored by fluorescence polarization (FP). An optimized concentration of a fluorophore-labeled peptide (determined via serial dilution and which yields a polarization baseline between 0 and 50 mP) was solubilized in FP buffer (150 mM NaCl, 20 mM sodium phosphate, 0.05% Tween-20, pH 7.4). MHC-I proteins and fluorophore-labeled peptides were directly added to the plate to 100 μL per well to avoid extended incubation and loss of data. The kinetic association was monitored for 2-12 hours, and polarization measurements were recorded every 28-105 seconds. The WT or open pMHC-I concentration remained constant across experiments at 200 nM, except for the MHC titration assays. Excitation and emission values used to monitor the fluorescence of TAMRA-labeled peptides were 531 and 595 nm, and FITC-labeled peptides were 475 and 525 nm. All experiments were performed at RT in triplicates.

For IC₅₀ competition assays, a serial dilution of competitor peptide was added to 200 nM WT or open pMHC-I and the optimal concentration of fluorophore-labeled peptide. Kinetic association measurements were collected as previously described. Non-linear regression fitting allowed calculating plateau polarization (mP) values for each kinetic curve. Log transformed values of each peptide concentration were plotted against the plateau mP value, and an IC₅₀ curve was fit.

Raw parallel (In) and perpendicular emission intensities (I_{⊥}) were collected and converted to polarization (mP) values using the equation 1000*[(I_{II}−(G*I_{⊥}))/(I_{II}+(G*I_{⊥}))]. An optimized G-factor was determined to be 0.33 for TAMRA-labeled peptides and 0.4 for FITC-labeled peptides in calculating baseline fluorescence and overall FP. The data analysis method was adapted and data fitting was performed in GraphPad Prism v9⁽⁵⁸⁾.

Biotinylation and tetramer formation. Biotinylation and tetramer formation of the WT and open HLA-A*02:01/KILGFVFβFV proteins were performed as previously described⁽²⁸⁾. The BSP-tagged proteins were biotinylated using the BirA biotin-protein ligase bulk reaction kit (Avidity), according to the manufacturer's instructions. Biotinylated molecules were washed using Amicon Ultra centrifugal filter units with a 100 kDa membrane cut-off, and the level of biotinylation was evaluated by SDS-PAGE gel shift assay in the presence of excess streptavidin.

Biotinylated WT and open HLA-A*02:01/KILGFVFβFV were mixed with 10-fold molar excess of the NYESO-1 peptide variants, SLLMWITQV, SLLMWITQC, and SLLMWITQA. Each reaction was incubated 2 hours at room temperature and the peptide exchange reactions were confirmed by DSF. NYESO-1 peptide-loaded HLA-A*02:01 molecules were prepared at a final concentration of 2 mg/mL. Streptavidin-PE (Agilent Technologies, Inc.) at 4:1 monomer/streptavidin molar ratio was added to pMHC-I/β₂m over 10-time intervals every 10 mins at RT in the dark. The resulting pMHC-I/β₂m tetramers can be stored at 4° C. for up to 4 weeks.

1G4 TCR lentivirus production. Lenti-X 293T cells (Takara) were cultured in DMEM (Gibco), 10% FBS (Gibco), and Glutamax (Gibco) and were plated one day before transfection. Cells were transfected at a confluency of 80-90% with TransIT-293 (Mirus) using pMD2.G (Addgene #12259, gift from Didier Trono), psPAX2 (Addgene #12260, gift from Didier Trono), and pSFFV-1G4. Virus-containing media was collected 24- and 48-hours post-transfection, clarified by centrifugation at 500 g for 10 min, and incubated with Lenti-X concentrator (Takara) for at least 24 hours. Virus was pooled and concentrated 50-100x, resuspended in PBS, aliquoted, and stored at −80° C. for subsequent T cell infections.

Primary human T cell tetramer staining. The studies involving human participants were reviewed and approved by the University of Pennsylvania review board. Written informed consent to participate in this study was provided by the participants. Healthy donor T cells were processed by the Human Immunology Core at the University of Pennsylvania by magnetic separation of CD8+ T cells. Cells were cultured in Advanced RPMI (Gibco), 10% heat inactivated FBS (Gibco), Glutamax (Gibco), penicillin/streptomycin (Gibco), and 10 mM HEPES (Quality Biological), supplemented with 300 U/mL recombinant IL-2 (NCI Biological Resources Branch). T cells were maintained at ˜1 million cells/mL and were activated with a 1:1 ratio of Dynabeads Human T-Activator CD3/CD28 beads (Gibco) for 48 hours. 24 hours after initial activation, cells were either left untransduced or transduced with lentivirus expressing the 1G4 TCR. Cells were debeaded by magnetic separation and expanded in the presence of IL-2. Transduction efficiency was determined by staining with an anti-Vβ13.1-APC antibody (Miltenyi Biotec.), typically greater than 50%. Cells were cryopreserved with CryoStor CS10 (StemCell Technologies). Thawed T cells were recovered and regrown in IL-2-containing complete media for −3 days prior to staining. Cells were harvested and washed with PBS/1% BSA/2 mM EDTA with 5 μg/mL PE-conjugated tetramer and incubated for 25 min at room temperature with slight shaking. After two washes with an RPMI-based wash buffer containing 1% FBS, cells were resuspended in 1:1000 Sytox Blue diluted in wash buffer to distinguish dead cells. Samples were processed on an CytoFLEX LX and the data analyzed by FlowJo v10.8.1.

### Example 2 References

1. P. Parham, T. Ohta, Population Biology of Antigen Presentation by MHC Class I Molecules. Science 272, 67-74 (1996).
2. M. Wieczorek, et al., Major Histocompatibility Complex (MHC) Class I and MHC Class II Proteins: Conformational Plasticity in Antigen Presentation. Frontiers in Immunology 8 (2017).
3. P. J. Bjorkman, et al., Structure of the human class I histocompatibility antigen, HLA-A2. Nature 329, 506-512 (1987).
4. D. J. Barker, et al., The IPD-IMGT/HLA Database. Nucleic Acids Res, gkac1011 (2022).
5. M. Hamdahl, et al., Peptide-MHC class I stability is a better predictor than peptide affinity of CTL immunogenicity: Antigen processing. Eur. J. Immunol. 42, 1405-1416 (2012).
6. A. Bailey, et al., Selector function of MHC I molecules is determined by protein plasticity. Sci Rep 5, 14928 (2015).
7. D. N. Garboczi, D. T. Hung, D. C. Wiley, HLA-A2-peptide complexes: refolding and crystallization of molecules expressed in Escherichia coli and complexed with single antigenic peptides. Proc Natl Acad Sci USA 89, 3429-3433 (1992).
8. J. D. Altman, et al., Phenotypic analysis of antigen-specific T lymphocytes. Science 274, 94-96 (1996).
9. S. R. Hadrup, et al., “High-Throughput T-Cell Epitope Discovery Through MHC Peptide Exchange” in Epitope Mapping Protocols: Second Edition, Methods in Molecular Biology™., M. Schutkowski, U. Reineke, Eds. (Humana Press, 2009), pp. 383-405.
10. H. V. Truong, N. G. Sgourakis, Dynamics of MHC-I molecules in the antigen processing and presentation pathway. Current Opinion in Immunology 70, 122-128 (2021).
11. K. L. Rock, E. Reits, J. Neefjes, Present Yourself! By MHC Class I and MHC Class II Molecules. Trends in Immunology 37, 724-737 (2016).
12. P. Cresswell, A. L. Ackerman, A. Giodini, D. R. Peaper, P. A. Wearsch, Mechanisms of MHC class I-restricted antigen processing and cross-presentation. Immunological Reviews 207, 145-157 (2005).
13. P. A. Wearsch, P. Cresswell, The quality control of MHC class I peptide loading. Curr Opin Cell Biol 20, 624-631 (2008).
14. J. S. Blum, P. A. Wearsch, P. Cresswell, Pathways of Antigen Processing. Annu Rev Immunol 31, 443-473 (2013).
15. A. C. McShan, et al., Peptide exchange on MHC-I by TAPBPR is driven by a negative allostery release cycle. Nat Chem Biol 14, 811-820 (2018).
16. L. Li, M. Dong, X.-G. Wang, The Implication and Significance of Beta 2 Microglobulin: A Conservative Multifunctional Regulator. Chinese Medical Journal 129, 448-455 (2016).
17. A.-K. Binz, R. C. Rodriguez, W. E. Biddison, B. M. Baker, Thermodynamic and Kinetic Analysis of a Peptide-Class I MHC Interaction Highlights the Noncovalent Nature and Conformational Dynamics of the Class I Heterotrimer. Biochemistry 42, 4954-4961 (2003).
18. D. M. Gakamsky, P. J. Bjorkman, I. Pecht, Peptide Interaction with a Class I Major Histocompatibility Complex-Encoded Molecule: Allosteric Control of the Ternary Complex Stability. Biochemistry 35, 14841-14848 (1996).
19. H. G. Ljunggren, et al., Empty MHC class I molecules come out in the cold. Nature 346, 476-480 (1990).
20. T. N. M. Schumacher, et al., Direct binding of peptide to empty MHC class I molecules on intact cells and in vitro. Cell 62, 563-567 (1990).
21. B. Rodenko, et al., Generation of peptide-MHC class I complexes through UV-mediated ligand exchange. Nat Protoc 1, 1120-1132 (2006).
22. M. L. Fahnestock, I. Tamir, L. Narhi, P. J. Bjorkman, Thermal stability comparison of purified empty and peptide-filled forms of a class I MHC molecule. Science 258, 1658-1662 (1992).
23. M. Toebes, et al., Design and use of conditional MHC class I ligands. Nat Med 12, 246-251 (2006).
24. A. H. Bakker, et al., Conditional MHC class I ligands and peptide exchange technology for the human MHC gene products HLA-A1, -A3, -A11, and -B7. Proc Natl Acad Sci USA 105, 3825-3830 (2008).
25. J. J. Luimstra, et al., A flexible MHC class I multimer loading system for large-scale detection of antigen-specific T cells. J Exp Med 215, 1493-1504 (2018).
26. C. Hermann, L. M. Strittmatter, J. E. Deane, L. H. Boyle, The Binding of TAPBPR and Tapasin to MHC Class I Is Mutually Exclusive. The Journal of Immunology 191, 5743-5750 (2013).
27. G. I. Morozov, et al., Interaction of TAPBPR, a tapasin homolog, with MHC-I molecules promotes peptide editing. Proc Natl Acad Sci USA 113, E1006-E1015 (2016).
28. S. A. Overall, et al., High throughput pMHC-I tetramer library production using chaperone-mediated peptide exchange. Nature Communications 11, 1909 (2020).
29. H. Lan, et al., Exchange catalysis by tapasin exploits conserved and allele-specific features of MHC-I molecules. Nat Commun 12, 1-13 (2021).
30. Y. Sun, et al., Xeno interactions between MHC-I proteins and molecular chaperones enable ligand exchange on a broad repertoire of HLA allotypes. Science Advances 9, eade7151 (2023).
31. M. Bouvier, D. C. Wiley, Structural characterization of a soluble and partially folded class I major histocompatibility heavy chain/beta 2m heterodimer. Nat Struct Biol 5, 377-384 (1998).
32. S. K. Saini, et al., Not all empty MHC class I molecules are molten globules: Tryptophan fluorescence reveals a two-step mechanism of thermal denaturation. Molecular Immunology 54, 386-396 (2013).
33. E. Kurimoto, et al., Structural and functional mosaic nature of MHC class I molecules in their peptide-free form. Mol Immunol 55, 393-399 (2013).
34. Z. Hein, et al., Peptide-independent stabilization of MHC class I molecules breaches cellular quality control*. Journal of Cell Science, jcs.145334 (2014).
35. S. K. Saini, et al., Empty peptide-receptive MHC class I molecules for efficient detection of antigen-specific T cells. Sci. Immunol. 4, eaau9039 (2019).
36. R. Anjanappa, et al., Structures of peptide-free and partially loaded MHC class I molecules reveal mechanisms of peptide selection. Nat Commun 11, 1314 (2020).
37. R. J. Malonis, J. R. Lai, O. Vergnolle, Peptide-Based Vaccines: Current Progress and Future Challenges. Chem. Rev. 120, 3210-3229 (2020).
38. A. Sette, J. Sidney, HLA supertypes and supermotifs: a functional perspective on HLA polymorphism. Current Opinion in Immunology 10, 478-482 (1998).
39. J. Sidney, B. Peters, N. Frahm, C. Brander, A. Sette, HLA class I supertypes: a revised and updated classification. BMC Immunology 9, 1 (2008).
40. D. M. Gakamsky, et al., An Allosteric Mechanism Controls Antigen Presentation by the H-2Kb Complex. Biochemistry 38, 12165-12173 (1999).
41. A. Achour, et al., Structural Basis of the Differential Stability and Receptor Specificity of H-2Db in Complex with Murine versus Human 02-Microglobulin. Journal of Molecular Biology 356, 382-396 (2006).
42. A. A. Dombkowski, K. Z. Sultana, D. B. Craig, Protein disulfide engineering. FEBS Letters 588, 206-212 (2014).
43. Y. C. Liu, et al., A Molecular Basis for the Interplay between T Cells, Viral Mutants, and Human Leukocyte Antigen Micropolymorphism. J Biol Chem 289, 16688-16698 (2014).
44. H. E. Van Wart, A. Lewis, H. A. Scheraga, F. D. Saeva, Disulfide Bond Dihedral Angles from Raman Spectroscopy. Proc. Natl. Acad. Sci. U.S.A. 70, 2619-2623 (1973).
45. D. B. Craig, A. A. Dombkowski, Disulfide by Design 2.0: a web-based tool for disulfide engineering in proteins. BMC Bioinformatics 14, 346 (2013).
46. M. Wieczorek, et al., Major Histocompatibility Complex (MHC) Class I and MHC Class II Proteins: Conformational Plasticity in Antigen Presentation. Frontiers in Immunology 8 (2017).
47. K. Natarajan, et al., The Role of Molecular Flexibility in Antigen Presentation and T Cell Receptor-Mediated Signaling. Frontiers in Immunology 9 (2018).
48. C. Thomas, R. Tampe, Proofreading of Peptide-MHC Complexes through Dynamic Multivalent Interactions. Frontiers in Immunology 8 (2017).
49. A. C. McShan, et al., Molecular determinants of chaperone interactions on MHC-I for folding and antigen repertoire selection. Proc. Natl. Acad. Sci. U.S.A. 116, 25602-25613 (2019).
50. Z. Li, et al., The Mechanism of β2m Molecule-Induced Changes in the Peptide Presentation Profile in a Bony Fish. iScience 23, 101119 (2020).
51. A. Achour, et al., Structural Basis of the Differential Stability and Receptor Specificity of H-2Db in Complex with Murine versus Human β2-Microglobulin. Journal of Molecular Biology 356, 382-396 (2006).
52. G. Esposito, et al., The Controlling Roles of Trp60 and Trp95 in β2-Microglobulin Function, Folding and Amyloid Aggregation Properties. Journal of Molecular Biology 378, 887-897 (2008).
53. S. Ricagno, et al., DE loop mutations affect β2-microglobulin stability and amyloid aggregation. Biochemical and Biophysical Research Communications 377, 146-150 (2008).
54. G. R. Masson, et al., Recommendations for performing, interpreting and reporting hydrogen deuterium exchange mass spectrometry (HDX-MS) experiments. Nat Methods 16, 595-602 (2019).
55. A. van Hateren, et al., Direct evidence for conformational dynamics in major histocompatibility complex class I molecules. J Biol Chem 292, 20255-20269 (2017).
56. F. Sieker, S. Springer, M. Zacharias, Comparative molecular dynamics analysis of tapasin-dependent and -independent MHC class I alleles. Protein Sci 16, 299-308 (2007).
57. E. T. Abualrous, et al., F pocket flexibility influences the tapasin dependence of two differentially disease-associated MHC Class I proteins. European Journal of Immunology 45, 1248-1257 (2015).
58. R. Buchli, et al., Real-Time Measurement of in Vitro Peptide Binding to Soluble HLA-A*0201 by Fluorescence Polarization. Biochemistry 43, 14852-14863 (2004).
59. R. Buchli, et al., Development and Validation of a Fluorescence Polarization-Based Competitive Peptide-Binding Assay for HLA-A*0201A New Tool for Epitope Discovery. Biochemistry 44, 12491-12507 (2005).
60. J.-L. Chen, et al., Structural and kinetic basis for heightened immunogenicity of T cell vaccines. Journal of Experimental Medicine 201, 1243-1255 (2005).
61. S. A. Overall, et al., High throughput pMHC-I tetramer library production using chaperone-mediated peptide exchange. Nat Commun 11, 1909 (2020).
62. H. Li, K. Natarajan, E. L. Malchiodi, D. H. Margulies, R. A. Mariuzza, Three-dimensional structure of H-2Dd complexed with an immunodominant peptide from human immunodeficiency virus envelope glycoprotein 12011Edited by I. A. Wilson. Journal of Molecular Biology 283, 179-191 (1998).
63. D. Rozema, S. H. Gellman, Artificial Chaperone-assisted Refolding of Carbonic Anhydrase B. Journal of Biological Chemistry 271, 3478-3487 (1996).
64. V. Tugarinov, V. Kanelis, L. E. Kay, Isotope labeling strategies for the study of high-molecular-weight proteins by solution NMR spectroscopy. Nat Protoc 1, 749-754 (2006).
65. A. C. McShan, et al., Molecular determinants of chaperone interactions on MHC-I for folding and antigen repertoire selection. Proceedings of the National Academy of Sciences 116, 25602-25613 (2019).
66. K. Natarajan, et al., An allosteric site in the T-cell receptor Co domain plays a critical signalling role. Nat Commun 8, 15260 (2017).
67. Y. Shen, A. Bax, Protein backbone and sidechain torsion angles predicted from NMR chemical shifts using artificial neural networks. J Biomol NMR 56, 227-241 (2013).
68. F. Delaglio, et al., NMRPipe: a multidimensional spectral processing system based on UNIX pipes. J Biomol NMR 6, 277-293 (1995).
69. W. Lee, et al., Backbone resonance assignments and secondary structure of Ebola nucleoprotein 600-739 construct. Biomol NMR Assign 13, 315-319 (2019).
70. Schrödinger, LLC, The PyMOL Molecular Graphics System, Version 1.8 (2015).
71. M. C. F. Thomsen, M. Nielsen, Seq2Logo: a method for construction and visualization of amino acid binding motifs and sequence profiles including sequence weighting, pseudo counts and two-sided representation of amino acid enrichment and depletion. Nucleic Acids Research 40, W281-W287 (2012).
72. F. Sievers, et al., Fast, scalable generation of high-quality protein multiple sequence alignments using Clustal Omega. Mol Syst Biol 7, 539 (2011).

### Example 3: Nonclassical MHC Open MR1 Tetramers

Applicants evaluated whether the nonclassical MHC open MR1 tetramers can function similarly to the commercially available wild-type MR1 tetramer from the NIH tetramer facility and the wild-type MR1 tetramer generated in-house.

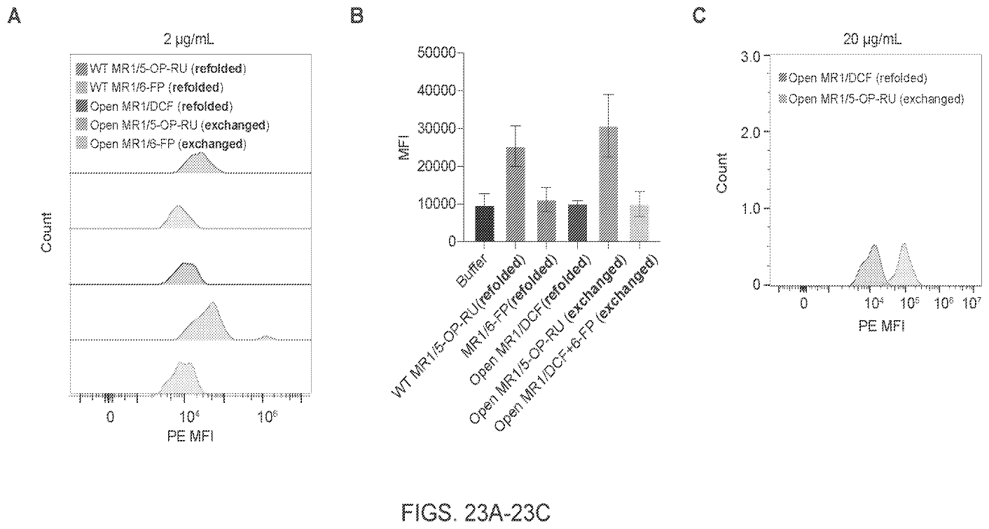

Exchanged open MR1/5-OP-RU tetramers demonstrated a similar staining level compared to the commercially available refolded WT MR1/5-OP-RU tetramers at 2 ug/mL as shown in FIGS. 23A-B. Increasing the tetramer concentration using exchanged open MR1/5-OP-RU showed no increase in background staining (FIG. 23C). To prepare exchanged WT MR1 tetramer, refolded MR1/DCF was exchanged with 5-OP-RU or 6-FP in 10-fold molar excess (7 uM: 70 uM) and loaded in a 1:4 molar ratio with PE tetramers.

FIGS. 23A-C depict the identification of MAIT cells from PBMCs using open MR1 tetramers exchanged for 5-OP-RU. In FIGS. 23A-B, approximately 5×10⁵ human PBMCs were stained with 2 μg ml⁻¹ of Buffer (no tetramer), WT MR1/5-OP-RU (refolded, NIH), WT MR1/6-FP(refolded, NIH), open MR1/DCF, open MR1/5-OP-RU(exchanged) and open MR1/6-FP(exchanged) for 30 min at 4° C. in the dark.

As depicted in FIG. 23C, approximately 5×10⁵ human PBMCs were stained with 20 μg ml⁻¹ open MR1/DCF and open MR1/5-OP-RU(exchanged). Cells were co-stained and gated with CD8+BV605, CD161-FITC, and Valpha7.2 (BD) for 30 min at 4° C. Cells were then washed three times with 200 μl of FACS wash (2% fetal bovine serum in PBS) and resuspended in 200 μl of FACS wash before acquisition of data on a Cytoflex LX. Data were analyzed using FlowJo analysis software. All tests are in technical triplicates.

Open MR1 loaded with vitamin B2 metabolite 5-(2-oxopropylideneamino)-6-d-ribitylaminouracil (5-OP-RU) efficiently stains all human MAIT cells in the CD8+ T cells comparable to the NIH and in-house WT MR1/5-OP-RU.

5-OP-RU was synthesized in-house from 5-N-RU using a previously established method (McShan, A. C. et al. TAPBPR employs a ligand-independent docking mechanism to chaperone MR1 molecules. Nat. Chem. Biol. 1-10 (2022) doi:10.1038/s41589-022-01049-9). The precursor compound 5-N-RU was provided by E. Adams (University of Chicago).

Approximately 2×10⁵ human CD8+ T cells were stained with PE and BUV395 streptavidin tetramerized with NIH, open, or WT MR1/5-OP-RU at 2 ug/mL for 30 mins on ice in the dark with slight shaking. Cells were co-stained with anti-human CD8α BV605, APC anti-human TCR Vu7.2 antibody, FITC anti-human CD161 antibody, and SYTOX™ Blue Dead Cell Stain in PBS/1% BSA/2 mM EDTA staining buffer. Cells were then washed three times with 200 uL RPMI-based wash buffer containing 1% FBS before acquiring data on a CytoFLEX LX (Beckman Coulter). The data were analyzed by FlowJo v10.8.1.

FIG. 18 depicts CD8+ T cells co-staining. Cells were stained and sorted by anti-human TCR Vα7.2 antibody, anti-human CD161 antibody, double negative NIH MR1/6-FP, NIH MR1/5-OP-RU, open MR1/5-OP-RU, and WT MR1/5-OP-RU.

FIG. 19 depicts flow cytometry gating strategy of CD8+ T cells. Primary human CD8+ T cells were gated by side and forward scatter (SSC-A and FSC-A) followed by single cell isolation (FSC-A versus FSC-H plot). Gating for live CD8+ cells was determined by Sytox blue staining and an anti-human CD8α antibody, and TRAV1-2+ MAIT cells were determined by staining with an anti-human TCR Vu7.2 antibody and anti-human CD161 antibody. Gates are shown in black, and the percentages of events are gated in parentheses. The acquisition was performed on CytoFLEX LX (Beckman Coulter), and the data were analyzed by FlowJo v10.8.1.

### Example 4: Application of N-Terminally Tagged Open Beta-2-Microglobulin—Generation of a Modular HLA Plug-and-Display System

Many CD8⁺ T cells found in the human body express a polyclonal and highly specific antigen receptor, called a T cell receptor (TCR) on the cell surface. These TCRs bind to the class I major histocompatibility complex (MHC-I) displaying an antigenic peptide and recognize the peptide in the context of the MHC-I molecules¹. This interaction is the first of many interactions necessary to kickstart the adaptive immune system². TCRs have a low avidity and fast off-rates for MHC-I complexes as a result of thymic selection^{1,3}, as such monomeric MHC-I complexes are not sufficient to detect antigen-specific T cells for the development of therapeutics against various infectious diseases, allergies, or cancers. More than two decades ago, MHC tetramers were introduced for the detection of antigen-specific T cells. These MHC tetramers showed an increased avidity for their cognate TCRs and have been successfully used since their introduction to detect, isolate, and study antigen-specific T cells⁴. However, one drawback of the current MHC tetramer technology is that in order to study multiple allotypes—of which for humans exist over 35,000⁵—one must prepare or purchase MHC-I heavy chain constructs with a biotin substrate protein (BSP-tag) located on the C-terminus of each respective heavy chain. The addition of a BSP-tag can prevent the proper formation of crystals in x-ray crystallography limiting the use of tagged constructs in structural studies⁶, and thus increase the cost of research as two constructs are required, one tagged and another without tags for functional and structural studies respectively. Thus, Applicants aim to add the BSP-tag, as well as SpyTag⁷ to the invariant light chain, beta-2-microglobulin (β₂m) using the open platform, this to Applicants' knowledge is the first attempt at the addition of a BSP or SpyTag onto β₂m effectively creating a system in which one must simply swap the tagged or untagged β₂m variants depending on their needs.

As a pilot study, the ectodomain of open A*02:01 (G120C) heavy chain and β₂m (H31C) tagged at the N-terminus with either BSP or SpyTag was refolded in the presence of excess peptide, KILGFVFβFV (KV9-βF), a previously developed conditional ligand for peptide exchange⁸. Prior attempts to add a BSP-tag to the C-terminus β₂m, were unsuccessful. Upon, structural analysis it was revealed that the addition of a BSP-tag at the C-terminus of the light chain likely interferes with α3 domain/β₂m packing of the ternary complex and completely abrogates binding of the heavy chain to the light chain. Thus, Applicants examine the addition of affinity-tags to the N-terminus of β₂m. Applicants include a 26 residue, Gly-rich linker containing a thrombin cleavage site between the affinity tag (BSP or SpyTag) and β₂m to prohibit unwanted interactions and interference between the functions of the tag and light chain.

Briefly, plasmids encoding the luminal domain of open A*02:01 (G120C) and open human β₂m (H31C) with either the N-terminally linked Biotin Substrate Peptide (BSP, LHHILDAQKMVWNHR) or SpyTag002 (VPTIVMVDAYKRYK) were transformed into E. coli cells. Cells were grown and harvested in the Luria-Broth medium, and inclusion bodies were pelleted and purified as previously described⁹. Ternary complexes were generated by in vitro refolding, a 100 mg mixture of open A02 and open β₂m (BSP or SpyTag) at a 1:3 molar ratio was slowly diluted over 4 hours in 500 mL of refolding buffer containing 0.4 M L-Arginine HCl, 100 mM Tris, 2 mM EDTA, 5 mM reduced L-glutathione, 0.5 mM oxidized L-glutathione, pH 8.0 supplemented with 5 mg of the peptide, KV9-βF. The refolding reaction proceeded for 4 days, and proteins were dialyzed into buffer containing 25 mM Tris, 150 mM NaCl, pH 8.0 and purified by size exclusion chromatography. Purified proteins were further confirmed in reduced and non-reduced conditions using sodium dodecyl sulfate-polyacrylamide (SDS-PAGE) gel electrophoresis.

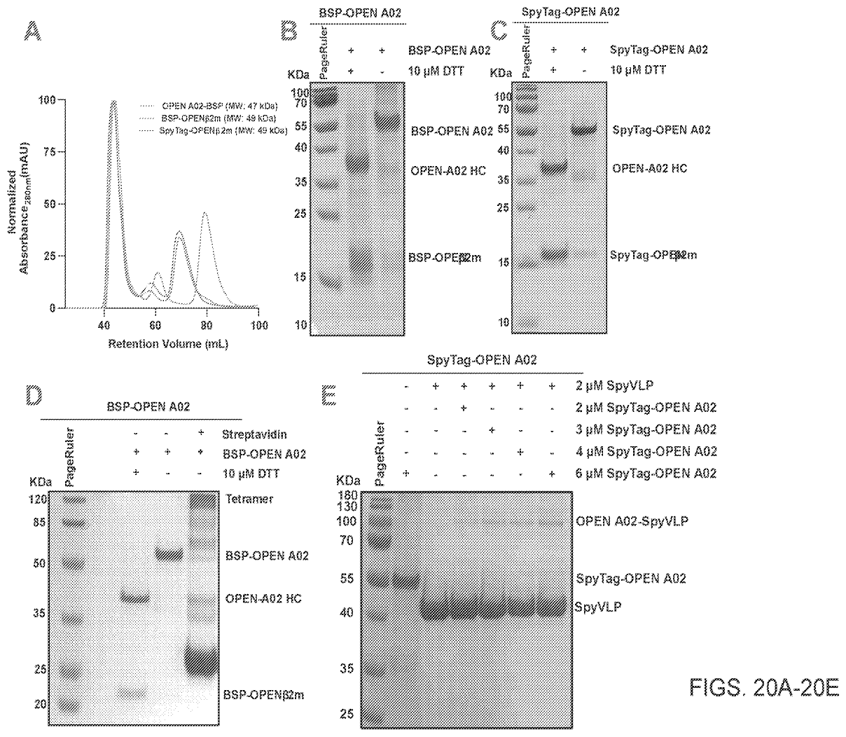

Altogether, Applicants refolded open A*02:01 (OPEN A02) using two different N-terminally tagged open β₂m molecules (Table 6). Both tagged variants were capable of forming a ternary complex, though at slightly lower yields than when the BSP-tag is fused to the C-terminus of open A*02:01 heavy chain (FIG. 20A). Reducing/non-reducing SDS-PAGE analysis confirms disulfide-linked complex formation (FIG. 20B-C) with BSP and SpyTag-open β₂m. Next, performed tetramerization of the BSP-OPEN A02 by conjugating the purified complex to streptavidin, tetramer formation was verified by use of SDS-PAGE analysis (FIG. 20D). The purified SpyTag-OPEN A02 was then conjugated to the SpyCatcher003-mi3 VLP to generate the SpyTag-OPEN A02:SpyCatcher003-mi3 (OPEN A02-SpyVLP) at various ratios (FIG. 20E).

FIGS. 20A-20E depict that N-terminally tagged β₂m are capable of complex formation. (A) SEC profile showing an overlay of open A02 with a BSP-tag fused to the C-terminus of the heavy chain (gray), a BSP-tag fused to the N-terminus of β₂m (pink), and a SpyTag fused to the N-terminus of β₂m (blue). (B) Coomassie-stained SDS-PAGE of open A02/BSP-open β₂m/KV9-βF complex under reducing and non-reducing conditions. (C) Coomassie-stained SDS-PAGE of open A02/SpyTag-open β₂m/KV9-βF complex under reducing and non-reducing conditions. (D) Conjugation of Streptavidin with open A02/BSP-open β₂m/KV9-βF. (E) Conjugation of SpyCatcher003-mi3 with open A02/SpyTag-open β₂m/KV9-βF at various ratios. Reactions were performed at 4° C. overnight and analyzed using SDS-PAGE with Coomassie staining.

TABLE 6 provides sequences of the N-terminally tagged β₂m.

### Example 4 References

1. Szeto, C., Lobos, C. A., Nguyen, A. T. & Gras, S. TCR Recognition of Peptide-MHC-I: Rule Makers and Breakers. Int. J. Mol. Sci. 22, 68 (2020).
2. Smith-Garvin, J. E., Koretzky, G. A. & Jordan, M. S. T Cell Activation. Annu. Rev. Immunol. 27, 591-619 (2009).
3. Arstila, T. P. et al. A Direct Estimate of the Human ap T Cell Receptor Diversity. Science 286, 958-961 (1999).
4. Altman, J. D. et al. Phenotypic Analysis of Antigen-Specific T Lymphocytes. Science 274, 94-96 (1996).
5. Barker, D. J. et al. The IPD-IMGT/HLA Database. Nucleic Acids Res. 51, D1053-D1060 (2023).
6. Wojtkowiak, A., Witek, K., Hennig, J. & Jaskolski, M. Two high-resolution structures of potato endo-1,3-β-glucanase reveal subdomain flexibility with implications for substrate binding. Acta Crystallogr. D Biol. Crystallogr. 68, 713-723 (2012).
7. Reddington, S. C. & Howarth, M. Secrets of a covalent interaction for biomaterials and biotechnology: SpyTag and SpyCatcher. Curr. Opin. Chem. Biol. 29, 94-99 (2015).
8. Sun, Y. et al. Xeno interactions between MHC-I proteins and molecular chaperones enable ligand exchange on a broad repertoire of HLA allotypes. Sci. Adv. 9, eade7151 (2023).
9. Li, H., Natarajan, K., Malchiodi, E. L., Margulies, D. H. & Mariuzza, R. A. Three-dimensional structure of H-2Dd complexed with an immunodominant peptide from human immunodeficiency virus envelope glycoprotein 120. J. Mol. Biol. 283, 179-191 (1998).

### Example 5: Application of Open MR1-a Loadable System to Screen Cancer Metabolites

The monomorphic nonclassical MHC-I-related protein 1 (MR1), which is structurally similar to classical MHC molecules, is found in all nucleated cells. While classical MHC-I binds to peptide antigens, MR1 presents small molecule ligands derived from the endogenous and exogenous metabolome to innate-like mucosal-associated invariant T (MAIT) and MR1-restricted T (MR1T) lymphocytes^{1,2}. Applicants have developed an integrated approach using biophysics, biochemistry, solution nuclear magnetic resonance (NMR) spectroscopy, computational biology, and immunological assays to characterize MR1/metabolite assembly, including interactions with molecular chaperones^{3,4}. Recently, several studies have also identified MAIT or MR1T clones exhibiting MR1 dependent cytotoxicity activity against tumor cells^{5,6}. These studies imply the existence of endogenous cancer metabolites including tumor association antigens or oncometabolites. Therefore, Applicants aim to elucidate how aberrant metabolomes are displayed to the immune system by first identifying and validating MR1 ligands from cancer metabolomes.

The Open MR1, consisting of MR1 (G120C, C262S) heavy chain and β₂m (H31C) was refolded in the presence of 20 mg diclofenac (DCF), a non-covalent ligand that was not previously refolded in vitro to yield stable ternary complexes. Refolded open MR1 underwent three dialysis changes of 1:25 dilutions, leading to a final buffer exchange with a dilution of more than 1:10,000. DCF-loaded MR1 proteins were then purified using size exclusion chromatography and confirmed through SDS-PAGE gel and differential scanning fluorimetry. A chemically stable fluorescent analog of the potent MAIT cell antigen JYM207^{7,8} 5-(2-oxopropylideneamino)-6-D-ribitylaminouracil (5-OP-RU) was generated in-house. Previous studies have shown that this fluorescent ligand competitively inhibits the activation of MAIT cells by 5-OP-RU, which specifically binds to MR1⁷. To optimize fluorescence polarization (FP), a direct binding assay between JYM20 and MR1 was conducted to determine the minimum concentration of MR1 and JYM20 required for a maximal signal, as well as the buffer conditions. The FP signal is generated by polarized excitation wavelengths, resulting in depolarized emission when JYM20 is free in solution (with low FP value). Conversely, when JYM20 is bound to the larger MR1 protein, the emitted light is mostly polarized due to the significantly slower tumbling speed of the MR1-JYM20 complex (with high FP value). Because DCF is not a covalent binding ligand, the DCF-loaded open MR1 and JYM20 can together screen a wide range of cancer metabolites for MR1.

Briefly, a buffer consistent of 150 mM NaCl, 20 mM sodium phosphate, 0.05% Tween-20, pH 7.4 was used since Applicants don't need nonphysiologically acidic environment to break the Schiff base. A serial dilution of cancer metabolites in a range of 60-0.06 mM, which were selected from the preliminary in silico screening, was added to 200 nM MR1 and 10 nM JYM20 in a final volume of 100 μL. The kinetic association was monitored for 16 hours, and polarization measurements were recorded every 186 seconds. Excitation and emission values used to monitor the fluorescence of TAMRA-labeled peptides were 531 and 595 nm. All experiments were performed in triplicates at room temperature. Raw parallel (III) and perpendicular emission intensities (I⊥) were collected and converted to polarization (mP) values using the equation 1000*[(III−(G*I⊥))/(III+(G*I⊥))]. An optimized G-factor was determined to be 0.33 for TAMRA-labeled peptides in calculating baseline fluorescence. Non-linear regression fitting of one phase association allowed calculating plateau polarization (mP) values for each kinetic curve. Log transformed values of each peptide concentration were plotted against the plateau mP value, and an IC50 curve was fit using log (inhibitor) vs. response (three parameters) curve from GraphPad Prism v9⁹.

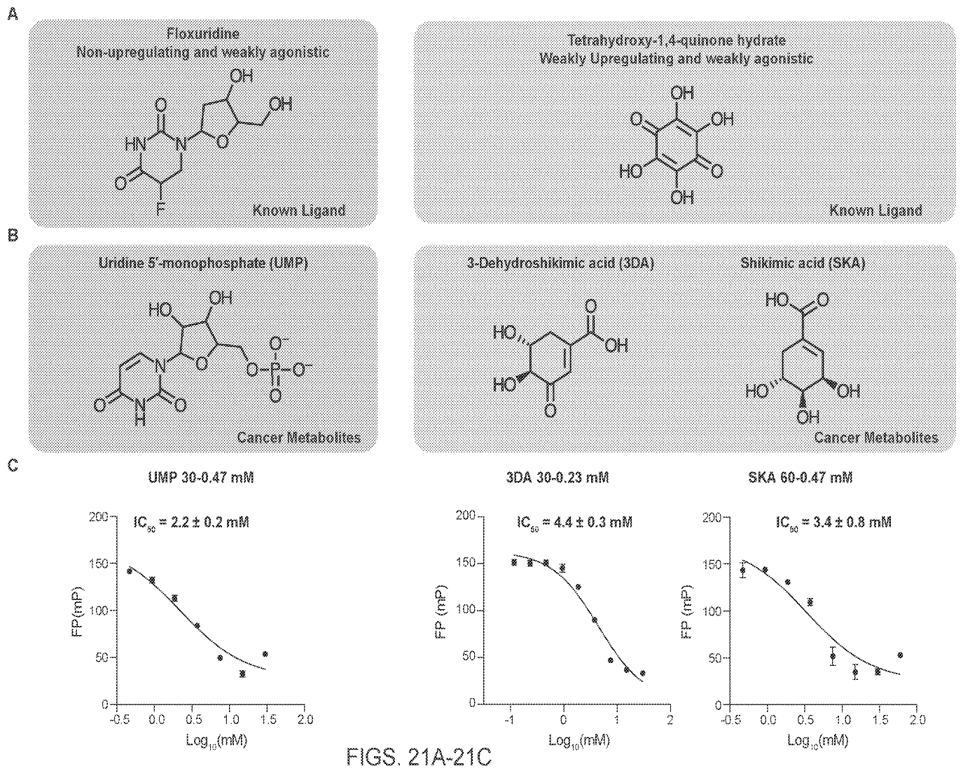

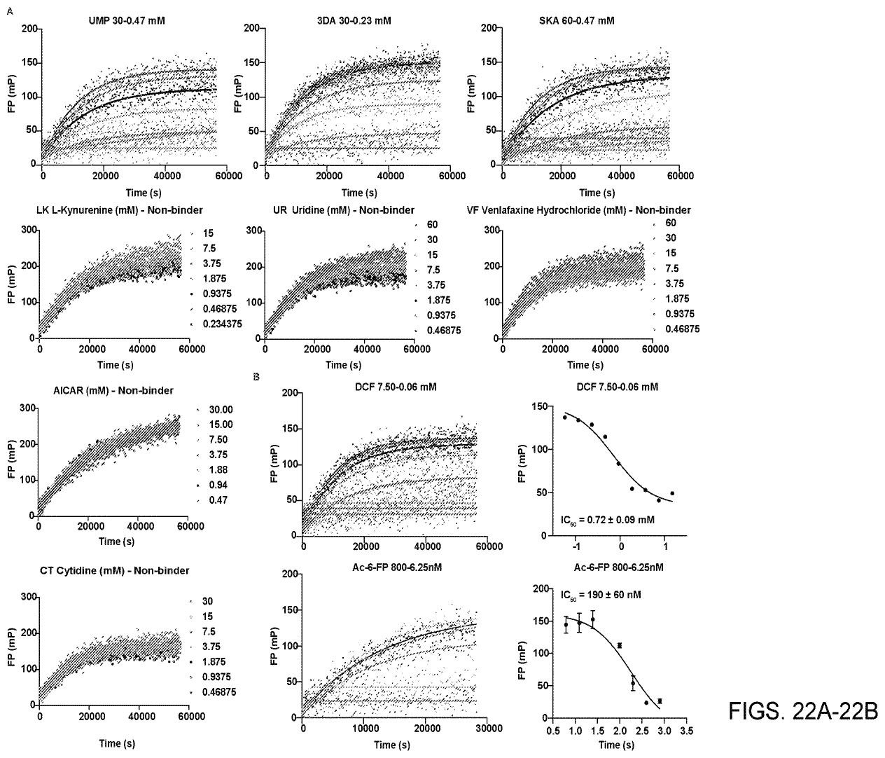

Collectively, Applicants screened 13 cancer metabolites (Table 7). 8 compounds were soluble up to at least 30 mM, allowing a wide range of concentration screening. 3 out of 8 cancer metabolites demonstrated similar structure to the known compound floxuridine and tetrahydroxy-1,4-quinone hydrate (FIG. 21A-B). Uridine 5′-monophosphate (UMP), 3-Dehydroshikimic acid (3DA), and shikimic acid (SKA), like DCF, exhibited low-millimolar relative binding affinities (IC₅₀) to MR1 (FIG. 21C, FIGS. 22 A-B). These compounds were eluted from the metabolomic profiling of the tumor cells and participating in important pathway and upregulating MR1 expression¹⁰.

FIGS. 21A-21C depict three cancer metabolites bind to MR1 at low millimolar binding affinities. A-B. Chemical structure of MR1-restricted known ligand floxuridine and tetrahydroxy-1,4-quinone hydrate and cancer metabolites Uridine 5′-monophosphate (UMP), 3-Dehydroshikimic acid (3DA), and shikimic acid (SKA). Chemicals sharing the same color background show structure similarity. C. Titration curves of cancer metabolites binding to MR1. Each data point represents plateau polarization (mP) values from three independent experiments performed in triplicate. Mean values are plotted with SD represented in error bars.

FIGS. 22A-22B depict competitive Binding of cancer metabolites and known ligands to open MR1. Ligand exchange measured by fluorescence polarization (miP) of 10 nM JMY20 and 200 nM DCF-loaded open MR1 in the presence of a serial dilution of cancer metabolites, as indicated. Individual traces were fit to a mono-exponential association model to determine plateau polarization (mP) values plotted in FIG. 21C and FIG. 22B. Results of three replicates (mean values) are plotted. B. Competitive binding of JMY20 to MR1 in the presence of known ligands DCF and acetyl-6-formylpterin (Ac-6-FP), and the IC₅₀ profiles extracted from the association profiles. IC₅₀ values were determined by fitting a log (inhibitor) vs. response (three parameters) curve. Results of three replicates (mean±SD) are plotted.

TABLE 7 provides a summary of the cancer metabolites.

### Example 5 References

1. Corbett, A. J., Awad, W., Wang, H. & Chen, Z. Antigen Recognition by MR1-Reactive T Cells; MAIT Cells, Metabolites, and Remaining Mysteries. Front. Immunol. 11, 1961 (2020).
2. Kjer-Nielsen, L. et al. MR1 presents microbial vitamin B metabolites to MAIT cells. Nature 491, 717-723 (2012).
3. McShan, A. C. et al. TAPBPR employs a ligand-independent docking mechanism to chaperone MR1 molecules. Nat. Chem. Biol. 1-10 (2022) doi:10.1038/s41589-022-01049-9.
4. Sun, Y. et al. Xeno interactions between MHC-I proteins and molecular chaperones enable ligand exchange on a broad repertoire of HLA allotypes. Sci. Adv. 9, eade7151 (2023).
5. Crowther, M. D. et al. Genome-wide CRISPR-Cas9 screening reveals ubiquitous T cell cancer targeting via the monomorphic MHC class I-related protein MR1. Nat. Immunol. 21, 178-185 (2020).
6. Lepore, M. et al. Functionally diverse human T cells recognize non-microbial antigens presented by MR1. eLife 6, e24476 (2017).
7. McWilliam, H. E. G. et al. Endoplasmic reticulum chaperones stabilize ligand-receptive MR1 molecules for efficient presentation of metabolite antigens. Proc. Natl. Acad. Sci. 117, 24974-24985 (2020).
8. Wang, C. J. H. et al. Quantitative affinity measurement of small molecule ligand binding to major histocompatibility complex class-I-related protein 1 MR1. J Biol. Chem. 298, 102714 (2022).
9. Buchli, R. et al. Real-Time Measurement of in Vitro Peptide Binding to Soluble HLA-A*0201 by Fluorescence Polarization. Biochemistry 43, 14852-14863 (2004).
10. Gwynne, W. D. et al. Cancer-selective metabolic vulnerabilities in MYC-amplified medulloblastoma. Cancer Cell 40, 1488-1502.e7 (2022).

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A complex comprising
(a) a protein encoding any one of (i) a dsMHC sequence of any one of Tables 1-6 and/or (ii) a sequence in FIG. 2 or FIG. 9A with a G120 mutation and/or a H31 mutation and
(b) a peptide or a ligand capable of eliciting an immune response.

2. The complex of claim 1, wherein the peptide or ligand in (b) comprises an immune epitope.

3. The complex of claim 1, wherein the peptide or ligand in (b) is a small molecule metabolite, a peptide, or a lipid.

4. The complex of claim 3, wherein the small molecule metabolite is hypoxanthine, guanine, cytidine, guanosine, 5-aminoimidazole-4-carboxamide-1-β-D-ribofuranoside (AICAR), uridine 5′-monophosphate (UMP), shikimic acid, 3-dehydroshikimic acid, ruidine, L-kynurenine, venlafanxine, salicylic acid, salicyluric acid, guanosine-5′-monophosphate (GMP), adenosine 5′-monophosphate monohydrate (AMP), or cytidine 5′-monophosphate (CMP).

5. The complex of claim 2, wherein the epitope is a tumor-associated epitope or neoantigen.

6. The complex of any one of claims 1-5 wherein the peptide or ligand in (b) is from the Cancer Genome Atlas (TCGA) mutant epitope library (see, e.g., https://www.cancer.gov/about-nci/organization/ccg/research/structural-genomics/tcga).

7. A first nucleic acid encoding the protein of claim 1(a) and a second nucleic acid encoding the peptide of claim 1(b).

8. The first nucleic acid and second nucleic acid of claim 7, wherein the first nucleic acid and the second nucleic acid is a single nucleic acid.

9. A first vector encoding the first nucleic acid and a second vector encoding the second nucleic acid of claim 7.

10. A single-chain vector encoding the single nucleic acid of claim 8.

11. A pharmaceutical composition comprising the complex of any one of claims 1-6 or the nucleic acid of claim 7 or 8 or the vector of claim 9 or 10 and a pharmaceutically acceptable carrier.

12. A method of treating or preventing a disease in a subject in need thereof, comprising administering to the subject a therapeutically effective amount of the complex of any one of claims 1-6 or the nucleic acid of claim 7 or 8 or the vector of claim 9 or 10 or the pharmaceutical composition of claim 10.

13. The method of claim 12, wherein the disease is cancer.

14. Use of the complex of any one of claims 1-6 or the nucleic acid of claim 7 or 8 or the vector of claim 9 or 10 or the pharmaceutical composition of claim 11 in a monomer, tetramer, or multimer form to screen a panel of candidate peptides or ligands to identify relevant peptides or ligands disease targets and their receptors.

15. The use of claim 14, wherein the disease is cancer.

16. The use of claim 14, wherein the disease is autoimmunity.

17. The use of claim 13, wherein the disease is an infectious disease.
